(19) United States
(12) Patent Application Publication
(10) Pub. No.: US 2025/0221985 A1
(43) Pub. Date: Jul. 10, 2025

(54) Oxabicycloheptanes for Modulation of Immune Response

(71) Applicants: Lixte Biotechnology, Inc., Pasadena, CA (US); The United States of America as Represented by the Secretary Department of Health and Human Services, Bethesda, MD (US)

(72) Inventors: John S. Kovach, Pasadena, CA (US); Zhengping Zhuang, Bethesda, MD (US); Sze Chun Winson Ho, Bethesda, MD (US); Herui Wang, Bethesda, MD (US); Rongze Lu, Sunnyvale, CA (US)

(21) Appl. No.: 18/979,811

(22) Filed: Dec. 13, 2024

Related U.S. Application Data
(63) Continuation of application No. 16/467,721, filed on Jun. 7, 2019, now Pat. No. 12,168,008.
(60) Provisional application No. 62/545,373, filed on Aug. 14, 2017.
(60) Provisional application No. 62/465,001, filed on Feb. 28, 2017.
(60) Provisional application No. 62/497,949, filed on Dec. 8, 2016.

(51) Int. Cl. A61K 31/496; A61K 9/00; A61K 39/00; A61K 39/395; A61P 35/00

(52) U.S. Cl. CPC A61K 31/496; A61K 9/0019; A61K 39/3955; A61P 35/00; A61K 2039/54; A61K 2039/545

(57) ABSTRACT

The present invention provides a method of treating a subject afflicted with cancer comprising administering to the subject an effective amount of a PP2A inhibitor.

### CROSS REFERENCE TO RELATED APPLICATIONS

This Application is a continuation of U.S. application Ser. No. 16/467,721, filed Jun. 7, 2019, which is a U.S. National Phase application, filed under 35 U.S.C. § 371, of International Application No. PCT/US2017/065270, filed Dec. 8, 2017, which claims the benefit of U.S. Provisional Patent Application No. 62/497,949, filed Dec. 8, 2016, U.S. Provisional Patent Application No. 62/465,001, filed Feb. 28, 2017, and U.S. Provisional Patent Application No. 62/545,373, filed Aug. 14, 2017, each of which is hereby incorporated herein by reference in its entirety.

### BACKGROUND OF THE INVENTION

Protein phosphatase 2A (PP2A) is a ubiquitous serine/threonine phosphatase that dephosphorylates numerous proteins of both ATM/ATR-dependent and -independent response pathways (Mumby, M. 2007). Pharmacologic inhibition of PP2A has previously been shown to sensitize cancer cells to radiation-mediated DNA damage via constitutive phosphorylation of various signaling proteins, such as p53, γH2AX, PLK1 and Akt, resulting in cell cycle deregulation, inhibition of DNA repair, and apoptosis (Wei, D. et al. 2013).

Cantharidin, the principle active ingredient of blister beetle extract (Mylabris), is a compound derived from traditional Chinese medicine that has been shown to be a potent inhibitor of PP2A (Efferth, T. et al. 2005). Although cantharidin has previously been used in the treatment of hepatomas and has shown efficacy against multidrug-resistant leukemia cell lines (Efferth, T. et al. 2002), its severe toxicity limits its clinical usefulness. LB-100 is a small molecule derivative of cantharidin with significantly less toxicity. Previous pre-clinical studies have shown that LB-100 can enhance the cytotoxic effects of temozolomide, doxorubicin, and radiation therapy against glioblastoma (GBM), metastatic pheochromocytoma, and pancreatic cancer (Wei, D. et al. 2013; Lu, J. et al. 2009; Zhang, C. et al. 2010; Martiniova, L. et al. 2011). LB-100 is also undergoing a phase 1 study in combination with docetaxel for the treatment of solid tumors (Chung, V. 2013).

### SUMMARY OF THE INVENTION

The present invention provides a method of treating a subject afflicted with cancer comprising administering to the subject an effective amount of a PP2A inhibitor in combination with an effective amount of a checkpoint inhibitor, wherein the amounts when taken together are effective to treat the subject.

The present invention also provides a method of treating a subject afflicted with cancer and receiving a checkpoint inhibitor comprising administering to the subject of an amount of PP2A inhibitor effective to enhance treatment relative to the checkpoint inhibitor alone.

The present invention also provides a method of treating a tumor or cancer in a subject comprising administering to the subject an effective amount of a PP2A inhibitor in combination with an effective amount of a checkpoint inhibitor, wherein the amounts when taken together are effective to treat the tumor or cancer.

The present invention also provides a method of increasing a T-cell response to cancer cells in a subject afflicted with cancer comprising administering to the subject an amount of a PP2A inhibitor in combination with an effective amount of a checkpoint inhibitor effective to increase the T-cell response to the cancer cells.

The present invention also provides a method of increasing T cell activation in a subject afflicted with cancer comprising administering to the subject an effective amount of a PP2A inhibitor in combination with an effective amount of a checkpoint inhibitor so as to thereby increase the T cell activation.

The present invention also provides a method of inhibiting the function of CTLA-4 in T cells comprising administering to the T cells a PP2A inhibitor so as to thereby inhibit the function of CTLA-4.

The present invention also provides a method of inhibiting PD-1:PD-L1 interaction in T cells comprising administering to the T cells a PP2A inhibitor so as to thereby inhibit interaction of PD-1:PD-L1.

### BRIEF DESCRIPTION OF THE DRAWINGS

FIG. 1. Greatest change in size of indicator lesion in patients with measurable disease at entry.

FIG. 2. Duration of stability or partial response (red circle) of disease (number of cycles) for each patient in ascending order of entry onto study.

FIG. 3A. Inhibition of PP2A significantly enhance IFN-γ production in CD4 T cells. IFN gamma production from activated CD4 T cells with CD3/CD28 beads for 5 days in presence or absence of LB-100 at 40 nM. LB-100 was added or replaced on the 3^{rd} day.

FIG. 3B. Inhibition of PP2A significantly enhance IFN-γ production in CD4 T cells. IFN gamma production from activated CD4 T cells with CD3/CD28 beads for 5 days in presence or absence of LB-100 at different concentration. LB-100 was added or replaced on the 3^{rd} day.

FIG. 4A. Inhibition of PP2A significantly enhance CD4 T cell proliferation. Percentage of proliferated CD4 T cells with CD3/CD28 beads for 5 days in presence or absence of LB-100 at 1000 nM. LB-100 was added or added or replaced on the 3^{rd} day.

FIG. 4B. Inhibition of PP2A significantly enhance CD4 T cell proliferation. Percentage of proliferated CD4 T cells with CD3/CD28 beads for 5 days in presence or absence of LB-100 at different concentration. LB-100 was added or replaced on the 3^{rd} day.

FIG. 5A. Inhibition of PP2A significantly enhance CD4 T cell proliferation. Representative flow plot of proliferated CD4 T cells with CD3/CD28 beads for 5 days in the absence of LB-100. LB-100 was added or replaced on the 3^{rd} day.

FIG. 5B. Inhibition of PP2A significantly enhance CD4 T cell proliferation. Representative flow plot of proliferated CD4 T cells with CD3/CD28 beads for 5 days in the presence of LB-100 at 1000 nM. LB-100 was added or replaced on the 3^{rd} day.

FIG. 6A. Inhibition of PP2A significantly enhance co-stimulatory molecule OX40 expression on T cells. Percentage of OX40 expressing CD4 T cells with CD3/CD28 beads for 5 days in presence or absence of LB-100 at 1000 nM. LB-100 was added or replaced on the 3^{rd} day.

FIG. 6B. Inhibition of PP2A significantly enhance co-stimulatory molecule OX40 expression on T cells. Percentage of OX40 expressing CD4 T cells with CD3/CD28 beads for 5 days in presence or absence of LB-100 at different concentration. LB-100 was added or replaced on the 3^{rd} day.

FIG. 7A. Inhibition of PP2A enhances Tbet, a transcription factor to drive IFNγ production in CD4 T cells. Percentage of Tbet expressing CD4 T cells with CD3/Cd28 beads for 5 days in presence or absence of LB-100 at 1000 nM. LB-100 was added or replaced on the 3^{rd} day.

FIG. 7B. Inhibition of PP2A enhances Tbet, a transcription factor to drive IFNγ production in CD4 T cells. Percentage of proliferated CD4 T cells co-culture monocyte-derived dendritic cells for 5 days in presence or absence of LB-100 at different concentration with or without anti-PD1 antibody. LB-100 was added or replaced on the 3^{rd} day.

FIG. 8A. Enhanced proliferation of CD4 T cells with combination treatment. Percentage of proliferated CD4 T cells co-cultured with monocyte-derived dendritic cells for 5 days in presence or absence of LB-100 at 8 nM with or without anti-PD1 antibody. LB-100 was added or replaced on the 3^{rd} day.

FIG. 8B. Enhanced proliferation of CD4 T cells with combination treatment. Percentage of proliferated CD4 T cells co-cultured with monocyte-derived dendritic cells for 5 days in presence or absence of LB-100 at different concentration with or without anti-PD1 antibody. LB-100 was added or replaced on the 3^{rd} day.

FIG. 9A. Representative flow cytometry plot of CD4 T cell proliferation in control.

FIG. 9B. Representative flow cytometry plot of CD4 T cell proliferation in LB-100.

FIG. 9C. Representative flow cytometry plot of CD4 T cell proliferation in anti-PD-1.

FIG. 9D. Representative flow cytometry plot of CD4 T cell proliferation in LB-100+anti-PD-1.

FIG. 10A. Enhanced OX40 expression in CD4 T cells with combination treatment. Percentage of OX40 expressing CD4 T cells co-cultured with monocyte-derived dendritic cells for 5 days in presence or absence of LB-100 at 8 nM with or without anti-PD1 antibody at 0.05 nM. LB-100 was added or replaced on the 3^{rd} day.

FIG. 10B. Enhanced OX40 expression in CD4 T cells with combination treatment. Percentage of OX40 expressing CD4 T cells co-cultured with monocyte-derived dendritic cells for 5 days in presence or absence of LB-100 at different concentrations with or without anti-PD1 antibody. LB-100 was added or replaced on the 3^{rd} day.

FIG. 11A. Enhanced Tbet expression in CD4 T cells with combination treatment. Percentage of Tbet expressing CD4 T cells co-cultured with monocyte-derived dendritic cells for 5 days in presence or absence of LB-100 at 200 nM with or without anti-PD1 antibody. LB-100 was added or replaced on the 3^{rd} day.

FIG. 11B. Enhanced Tbet expression in CD4 T cells with combination treatment. Percentage of Tbet expression in CD4 T cells co-cultured with monocyte-derived dendritic cells for 5 days in presence or absence of LB-100 at different concentrations with or without anti-PD1 antibody. LB-100 was added or replaced on the 3^{rd} day.

FIG. 12. PP2A inhibitor reduced mouse B16 melanoma tumor growth significantly in mice treated every two days for 8 doses. Treatment was started on the same day of tumor implantation. Control—PBS, Low dose—0.16 mg/kg, Medium dose—0.32 mg/kg.

FIG. 13A. PP2A inhibitor increased CD4/8 effector cells in naïve mice. Low dose of LB treatment in vivo induced more CD8 (left) and CD4 (right) effector T cells in lymph node. 5 mice per group. Control—PBS, Low dose—0.16 mg/kg, Medium dose—0.32 mg/kg.

FIG. 13B. PP2A inhibitor increased CD4/8 effector cells in naïve mice. Representative flow cytometry plot of CD44+CD62L− CD8 (left) and CD4 (right) in FIG. 13A. Control—PBS, Low dose—0.16 mg/kg, Medium dose—0.32 mg/kg.

FIG. 14A. PP2A inhibitor reduced PD-1 expression on CD8 T cell in blood and spleen. Low dose of LB treatment in vivo reduced PD-1 expressing CD8+ T cells in blood.

FIG. 14B. PP2A inhibitor reduced PD-1 expression on CD8 T cell in blood and spleen. Medium dose of LB treatment in vivo reduced PD-1 expressing CD8+ T cells in spleen.

FIG. 15. PP2A inhibitor enhanced IFN-g production from human T cells. IFNγ production in the supernatant from CD4 T cells co-cultured with monocytes derived DC in presence of LB-100, or anti-PD-1 or combination (LB-100 and anti-PD-1).

FIG. 16A. PP2A inhibitor reduced PD-1 expression on human CD4 T cells. Percentage of PD-1 expressing CD4 T cells which were co-cultured with monocytes derived DC in presence of isotype control.

FIG. 16B. PP2A inhibitor reduced PD-1 expression on human CD4 T cells. Percentage of PD-1 expressing CD4 T cells which were co-cultured with monocytes derived DC in presence of LB-100.

FIG. 16C. PP2A inhibitor reduced PD-1 expression on human CD4 T cells. Percentage of PD-1 expressing CD4 T cells which were co-cultured with monocytes derived DC in presence of anti-PD-1.

FIG. 16D. PP2A inhibitor reduced PD-1 expression on human CD4 T cells. Percentage of PD-1 expressing CD4 T cells which were co-cultured with monocytes derived DC in presence of combination (LB-100 and anti-PD-1).

FIG. 17A. CD8+CD44+ effector T cells are increased with PP2A inhibitor LB-100 treatment. Percentage of CD8+CD44+T effector cells population in tumor draining lymph node from B16 tumor bearing mice treated with LB-100 or PBS. 5 mice per group.

FIG. 17B. CD8+CD44+ effector T cells are increased with PP2A inhibitor LB-100 treatment. Representative flow cytometry plot of data shown in FIG. 17A.

FIG. 18A. Increased CD44+CD62L− CD4 effector T cells in lymph node from B16 tumor bearing mice. Percentage of CD44+CD62L− CD4 effector T cells population in lymph node from B16 tumor-bearing mice treated with LB-100 or PBS. 5 mice per group.

FIG. 18B. Increased CD44+CD62L− CD4 effector T cells in lymph node from B16 tumor bearing mice. Representative flow cytometry plot of data shown in FIG. 18A.

FIG. 19A. Increased CD44+CD62L− CD8 effector T cells in lymph node from B16 tumor bearing mice. Percentage of CD44+CD62L− CD8 effector T cell population in lymph node from B16 tumor-bearing mice treated with LB-100 or PBS. 5 mice per group.

FIG. 19B. Increased CD44+CD62L− CD8 effector T cells in lymph node from B16 tumor bearing mice. Representative flow cytometry plot of data shown in FIG. 19A.

FIG. 20A. BALB/c mice were implanted with CT26 cells in their right thoracic flanks subcutaneously. After 13 days, mice with tumors reaching 30-100 mm³ in size were randomized and treated with PBS control, anti-PD-L1, LB-100, or combination (LB-100 and anti-PD-1) for 28 days. Individual tumor volume over time.

FIG. 20B. BALB/c mice were implanted with CT26 cells in their right thoracic flanks subcutaneously. After 13 days, mice with tumors reaching 30-100 mm³ in size were randomized and treated with PBS control, anti-PD-L1, LB-100, or combination (LB-100 and anti-PD-1) for 28 days. Median tumor volume over time.

FIG. 20C. BALB/c mice were implanted with CT26 cells in their right thoracic flanks subcutaneously. After 13 days, mice with tumors reaching 30-100 mm³ in size were randomized and treated with PBS control, anti-PD-L1, LB-100, or combination (LB-100 and anti-PD-1) for 28 days. Mouse survival over time.

FIG. 21A. About 60 days after initial inoculation, cured mice and CT26-naive control mice, were (re)inoculated with CT26 cells in their left flanks. Individual tumor volumes over time.

FIG. 21B. About 60 days after initial inoculation, cured mice and CT26-naive control mice, were (re)inoculated with CT26 cells in their left flanks. Median tumor volumes over time.

FIG. 22A. BALB/c mice were implanted with CT26 cells in their right thoracic flanks subcutaneously. After 11 days, mice with tumors reaching 30-100 mm³ in size, were randomized into four groups: Control, CD8 depletion, CD8 depletion+Combination (LB-100 and anti-PD-1), or Combination only (LB-100 and anti-PD-1). Mice in the depletion group were then given CD8 depleting antibodies. Two days later mice were then started on respective treatment. Individual tumor volume over time.

FIG. 22B. BALB/c mice were implanted with CT26 cells in their right thoracic flanks subcutaneously. After 11 days, mice with tumors reaching 30-100 mm³ in size, were randomized into four groups: Control, CD8 depletion, CD8 depletion+Combination (LB-100 and anti-PD-1), or Combination only (LB-100 and anti-PD-1). Mice in the depletion group were then given CD8 depleting antibodies. Two days later mice were then started on respective treatment. Median tumor volume over time.

FIG. 22C. BALB/c mice were implanted with CT26 cells in their right thoracic flanks subcutaneously. After 11 days, mice with tumors reaching 30-100 mm³ in size, were randomized into four groups: Control, CD8 depletion, CD8 depletion+Combination (LB-100 and anti-PD-1), or Combination only (LB-100 and anti-PD-1). Mice in the depletion group were then given CD8 depleting antibodies. Two days later mice were then started on respective treatment. Mouse survival over time.

FIG. 23A. BALB/C mice were inoculated subcutaneously with CT26 tumor cells in the right thoracic flanks and treated with control (PBS), LB-100, anti-PD-1, or combination (LB-100 and anti-PD-1), as described in FIGS. 22A-C. Tumor-infiltrating T cells were analyzed by flow cytometry 12 days after the start of treatment. Percentage of CD8+ tumor infiltrating T cells producing IFNg+ after 4 hours of PMA stimulation was increased in the combination group (*p=0.05).

FIG. 23B. BALB/C mice were inoculated subcutaneously with CT26 tumor cells in the right thoracic flanks and treated with control (PBS), LB-100, anti-PD-1, or combination (LB-100 and anti-PD-1), as described in FIGS. 22A-C. Tumor-infiltrating T cells were analyzed by flow cytometry 12 days after the start of treatment. Percentage of CD4+FoxP3+T-regulatory cells of CD45+ cells in the tumor was decreased in the LB-100 treatment group (**p<0.01).

FIG. 24A. BALB/c mice were inoculated with 0.5×10⁶ CT26 cells subcutaneously in the right thoracic flank. When tumors reached between 50-100 mm³ mice were randomized to four treatment groups and treated every 2 days for 4 weeks.

FIG. 24B. Left, individual tumor growth curves: control, LB-100, a-PD-1, and combination. Middle, mean tumor size over time. Right, cumulative survival over time.

FIG. 24C. Efficacy of PP2A inhibition with PD-1 blockade is dependent on CD8+ T cells. BALB/c mice were inoculated as in 24 A. When tumors reached 30-100 mm³, mice were temporarily depleted of CD8+ T cells and treated with combination.

FIG. 24D. Left, individual tumor growth curves: control, combination, CD-8 depletion only, and combination with CD8 depletion. Middle, mean tumor size over time. Right, cumulative survival over time. Data are representative of 2 independent experiments. *P<0.05, **P<0.01 and ****P<0.0001 (log-rank Mantel-Cox test).

FIG. 25A. BALB/c mice were inoculated with 0.5×10⁶ CT26 cells subcutaneously and treated. CR or naïve control mice were re-challenged about 60-days after initial implantation with 0.5×10⁶ CT26 cells in the left thoracic flank or in combination with 1.25×10⁵ 4T1 breast carcinoma cells in the mammillary fat pad. Mice (re)-challenged with CT26 alone demonstrated no growth of CT 26 tumors.

FIG. 25B. Left, individual tumor growth curves: naive, CR. Right, mean tumor size over time.

FIG. 25C. Quantitation of CT26 tumor volume 18 days after inoculation. (P<0.001, two tailed student t-test).

FIG. 25D. CR and naïve mice were (re)-challenged with CT26 and 4T1 tumor cells: naïve—CT26, CR—CT26, naïve—4T1, CR—4T1. Left, individual tumor growth curves. Right, mean tumor size over time.

FIG. 25E. Quantitation of CT26 and 4T1 tumor volume 18 days after inoculation. (P<0.0001, one way ANOVA with Tukey's multiple comparison test).

FIG. 25F. Picture of representative naïve and CR mouse following inoculation CT26 and 4T1 tumors.

FIG. 26A. Representative FACS plots of CD44 and CD62L in CD8+ T cells in the spleen.

FIG. 26B. Quantification of CD62-CD44+(of CD8+ T cells) in the spleen (n=4-5).

FIG. 26C. Quantification of CD62-CD44+(of CD8+ T cells) in tumor draining lymph nodes (n=4-5).

FIG. 26D. Representative FACS plots of CD8+CD3+ T cells as percentage of CD45+ cells.

FIG. 26E. Immune infiltrate analysis of CD3+ expressed as percentage of CD45+ cells (n=5). Error bars depict SEM. Data represents one of two experiments with five independently analyzed mice/group.

FIG. 26F. Immune infiltrate analysis of CD8+ expressed as percentage of CD45+ cells (n=5). Error bars depict SEM. Data represents one of two experiments with five independently analyzed mice/group.

FIG. 26G. Immune infiltrate analysis of CD4+ expressed as percentage of CD45+ cells (n=5). Error bars depict SEM. Data represents one of two experiments with five independently analyzed mice/group.

FIG. 26H. Ratio of CD8+ to CD4+ cells in tumor. Error bars depict SEM. Data represents one of two experiments with five independently analyzed mice/group.

FIG. 26I. CD8+ and CD44+ expressed as percentage of CD45+ cells in tumor. Error bars depict SEM. Data represents one of two experiments with five independently analyzed mice/group.

FIG. 26J. CD8+ and Ki67+ expressed as percentage of CD45+ cells in tumor. Error bars depict SEM. Data represents one of two experiments with five independently analyzed mice/group.

FIG. 26K. Expression of PD1+ in CD8+ cells in tumor. *P<0.05, (one way ANOVA with Tukey's multiple comparison test).

FIG. 26L. Expression of CD4+ cells in tumor. *P<0.05, (one way ANOVA with Tukey's multiple comparison test).

FIG. 27A. Representative FACS plots of FoxP3+ and CD4+ T cells in tumors.

FIG. 27B. Percentage of CD4+FoxP3+ T cells of total CD3+ cells.

FIG. 27C. Ratio of CD8+ to CD4+FoxP3+Treg cells in tumor (n=5).

FIG. 27D. Representative FACS plots of CD8+IFNγ+ T cells of CD45+ cells.

FIG. 27E. Percentage of CD8+IFNγ+ T cells of CD45+ cells.

FIG. 27F. Percentage of CD8+TNFα+ T cells of CD45+ cells.

FIG. 27G. Percentage of CD8+ double positive IFNγ+TNFα+ T cells of CD45+ cells.

FIG. 27H. Percentage of CD8+GranzymeB+ T cells of CD45+ cells.

FIG. 27I. Percentage of CD4+IFNγ+ of CD4+ T cells.

FIG. 27J. Summary of CD45+ immune cell subsets and CD45− cells as determined by FACS. Subsets are depicted as percentage of all acquired live events (right) and CD3+ cells (left); Diagram on the right: Non CD45−, CD3+, Non CD3+CD45 leukocytes; Diagram on the left: CD8, CD4-Treg, CD4-conv. *P<0.05, (one way ANOVA with Tukey's multiple comparison test). Error bars depict SEM. Data represents one of two experiments with five independently analyzed mice/group.

FIG. 28A. Flow cytometric analysis of activation and immune checkpoint markers of (A) CD4+ in the spleen of mice receiving LB-100 and/or aPD-1 treatment. In CD4+ T cells, unlike CD8+ T cells, there was no change in expression of CD62L-CD44+ expression. There was also no change in expression of immune check point markers: PD1, CTLA4, TIM3 and Ox40.

FIG. 28B. Flow cytometric analysis of activation and immune checkpoint markers of CD8+ lymphocytes in the spleen of mice receiving LB-100 and/or aPD-1 treatment. In CD8+ T cells, there was no change in expression of immune check point markers: PD1, CTLA4, TIM3 and Ox40.

FIG. 29A. Flow cytometric analysis of activation and immune checkpoint markers of CD4+ in the draining lymph node (dLN) of mice receiving LB-100 and/or aPD-1 treatment. In CD4+ T cells, unlike CD8+ T cells, there was no change in expression of CD62L-CD44+ expression. There was a small, but significant increase in PD-1 expression in aPD-1 treated groups, but LB-100 alone or in combination did not further alter PD-1 expression. There was no change in expression of other immune check point markers: CTLA4, TIM3 and Ox40. *P<0.05, **<P<0.01 (one way ANOVA with Tukey's multiple comparison test). Error bars depict SEM.

FIG. 29B. Flow cytometric analysis of activation and immune checkpoint markers of CD8+ lymphocytes in the draining lymph node (dLN) of mice receiving LB-100 and/or aPD-1 treatment. In CD8+ T cells, there was no change in expression of immune check point markers: PD1, CTLA4, TIM3 and Ox40. *P<0.05, **<P<0.01 (one way ANOVA with Tukey's multiple comparison test). Error bars depict SEM.

FIG. 30. Gating strategy for flow cytometric analysis of tumor infiltrating lymphocytes. SSC-FSC gate was used to exclude non-cellular debris, followed by exclusion of duplets by FSC-H-FSA-A gate. Fixable live-dead (L/D) stain was used to exclude dead cells. Live cells were then gated based on expression of CD45+ pan leukocyte marker. CD45− cells were considered as tumor cells. CD45+ cells were then phenotyped further based on CD3, CD8, CD4 expression. CD45+CD3+CD8+ cells were gated as CD8+ lymphocytes, while CD45+CD3+CD4+ cells were gated as CD4+ lymphocytes. Further, staining of the CD4+ and CD8+ subsets were then performed as indicated in the text.

FIG. 31A. The ratios of CD3+, CD8+, and CD4+ cells to CD45− tumor-resident cells were shown for each treatment group. There was an increase in CD3/tumor and CD8/tumor ratios in the combination group compared to control, while there was no change in CD4/tumor ratio.

FIG. 31B. The number of CD3+, CD8+ and CD4+ cell per gram of tumor weight were shown for each treatment group. A similar trend was seen as in FIG. 31A, but there were significant differences in CD3+ and CD8+ per gram tumor in aPD-1 treated group alone compared to control. There was a trend of further increase in CD3+ and CD8+/tumor for combination treatment, but there was no statistical significance. *P<0.05, ***<P<0.001 (one way ANOVA with Tukey's multiple comparison test). Error bars depict SEM.

FIG. 32A. Flow cytometric analysis of and immune checkpoint markers of CD4+ lymphocytes in tumors of mice receiving LB-100 and/or aPD-1 treatment. In CD4+ T cells, there was no change in expression of immune check point markers: TIM3, Ox40, CTLA4 and LAG3.

FIG. 32B. Flow cytometric analysis of and immune checkpoint markers of CD8+ lymphocytes in tumors of mice receiving LB-100 and/or aPD-1 treatment. In CD8+ T cells, there was no change in expression of immune check point markers: TIM3, Ox40, CTLA4 and LAG3.

FIG. 33A. Representative flow cytometry plot showing increased TNF-α+. Percentage displayed are of total CD3+.

FIG. 33B. Representative flow cytometry plot showing increased TNF-α+IFN-γ+ double positive. Percentage displayed are of total CD8+.

FIG. 33C. Representative flow cytometry plot showing increased GranzymeB+CD8 tumor infiltrating T-cells. Percentage displayed are of total CD3+ cells.

FIG. 34A. C57BL/6 mice were randomized into four treatment groups. 2.5×10⁵ B16F10 cells were inoculated 2 days after initiation of treatment subcutaneously in the right thoracic flank. Mice were treated every two days until survival endpoint.

FIG. 34B. Left, individual tumor growth curves: control, LB-100, a-PD-1, and combination. Right, mean tumor size over time.

FIG. 34C. Quantitation of B16 tumor volume 15 days after inoculation. (P<0.0001, one way ANOVA with Tukey's multiple comparison test)

FIG. 34D. Cumulative survival over time. *P<0.05, (log-rank Mantel-Cox test) Data are pooled from 2 independent experiments.

FIG. 34E. Representative images of hematoxylin-and-eosin staining of the skin and salivary gland of each treatment group (n=2-3 per group). Scale bars, 100 m.

FIG. 35. Representative images of hematoxylin-and-eosin staining of the pancreas, lung and stomach of each treatment group (n=2-3 per group). Scale bars, 100 m.

FIG. 36A. CD3 T cells were isolated from mice splenocytes and cultured with or without stimulation using immobilized anti-CD3 (10.ig/ml) and soluble anti-CD28 (2.ig/ml). PP2A enzymatic activity was measured after 3 hours of activation. PP2A activity was measured as relative to activated control in presence of LB-100 dose titration.

FIG. 36B. Flow cytometry analyzing AKT phosphorylated at Thr308 (p-AKT(T308)) or Ser473 (p-AKT(S473)) after 3 hours of stimulation in presence of LB-100 dose titration.

FIG. 36C. Flow cytometry analyzing phosphorylated S6 (p-S6) in presence of LB-100 dose titration. *P<0.05, ***P<0.001, (one way ANOVA with Tukey's multiple comparison test). Data are from one experiment representative of two independent experiments with similar results. Error bars depict SEM.

FIG. 37. AKT and mTORC signaling after 30 minutes of stimulation. Flow cytometry analyzing AKT phosphorylated at Thr308 (p-AKT(T308)), Ser473 (p-AKT(S473)) or phosphorylated S6 (p-S6) after 30 minutes of stimulation in presence of LB-100 dose titration. (one way ANOVA with Tukey's multiple comparison test). Data are from one experiment representative of two independent experiments with similar results. Error bars depict SEM.

FIG. 38A. Left, the % of CD4 cells positive for Foxp3. Right, representative flow cytometry data demonstrating decreased in % of Foxp3 cells with LB-100. Cells were gated on CD4+ cells.

FIG. 38B. Intracellular levels of GATA3 were measured with flow cytometry. Left, the % of CD4 cells positive for GATA3. Right, representative flow cytometry data demonstrating decreased in % of GATA3 cells with LB-100.

FIG. 38C. Ratio of GATA3+Th2 over Tbet+Th1 CD4 cells.

FIG. 38D. Intracellular production of IFN-γ was measured by flow cytometry. Left, the % of CD4 cells positive for IFN-γ in T_{H}1 and T_{H}2 conditions. Right, representative flow cytometry data demonstrating increased in % of IFN-γ cells with LB-100 in both T_{H}1 and T_{H}2 conditions.

FIG. 38E. TNF, IL2 and IFN-γ production in supernatant of naïve CD4+ T cells activated in T_{H}1 skewing conditions for 3 days.

FIG. 38F. TNF, IL2, IFN-γ and IL4 production in supernatant of naïve CD4+ T cells activated in T_{H}2 skewing conditions for 3 days. Cytokine levels were adjusted to absolute cell number. *P<0.05, **P<0.01, ***P<0.001, (one way ANOVA with Tukey's multiple comparison test). Data are from one experiment representative of two independent experiments with similar results. Error bars depict SEM.

FIG. 39A. DCs were induced from purified monocytes by culturing in IL4 and GM-CSF for 7 days. 10⁵ purified CFSE labelled CD4+ T cells were then co-cultured with 10⁴ allogenic DCs in the presence of a titration of LB-100 in duplicates or triplicates for 5 days. LB-100 was replenished on day 3. Supernatants were collected on day 5 and measured for IFN-γ production. FACS analysis was performed on the cultured cells.

FIG. 39B. In vitro proliferation of CD4+ T cells in presence of LB-100 dose titration, measured by dilution of the cytosolic CFSE. Left, the % of cells divided was plotted against concentration of LB-100. Right, representative flow cytometry data demonstrating increased in % cells divided at 1 uM of LB-100.

FIG. 39C. IFN-α production was measured at day 5, demonstrating a dose dependent increase in IFN-α secretion with LB-100.

FIG. 39D. Intracellular staining of T-bet was performed in CD4+ T cells after 5 days of co-culture. Percentage of CD4+ Tbet+(of CD4+ cells) against LB-100 concentration.

FIG. 39E. (E) IFN-γ production in cells treated with isotype control, LB-100 and/or Nivolumab, demonstrating a synergistic response to combination treatment. *P<0.05, **P<0.01, ***P<0.001 (one way ANOVA with Tukey's multiple comparison test). Data are from one experiment representative of two independent experiments with similar results. Error bars depict SEM.

### DETAILED DESCRIPTION OF THE INVENTION

The present invention provides a method of treating a subject afflicted with cancer comprising administering to the subject an effective amount of a PP2A inhibitor in combination with an effective amount of a checkpoint inhibitor, wherein the amounts when taken together are effective to treat the subject.

The present invention also provides a method of treating a subject afflicted with cancer and receiving a checkpoint inhibitor comprising administering to the subject of an amount of PP2A inhibitor effective to enhance treatment relative to the checkpoint inhibitor alone.

The present invention also provides a method of treating a tumor or cancer in a subject comprising administering to the subject an effective amount of a PP2A inhibitor in combination with an effective amount of a checkpoint inhibitor, wherein the amounts when taken together are effective to treat the tumor or cancer.

The present invention also provides a method of increasing a T-cell response to cancer cells in a subject afflicted with cancer comprising administering to the subject an amount of a PP2A inhibitor in combination with an effective amount of a checkpoint inhibitor effective to increase the T-cell response to the cancer cells.

The present invention also provides a method of increasing T cell activation in a subject afflicted with cancer comprising administering to the subject an effective amount of a PP2A inhibitor in combination with an effective amount of a checkpoint inhibitor so as to thereby increase the T cell activation.

In some embodiments, the amount of the compound and the amount of the checkpoint inhibitor are each periodically administered to the subject.

In some embodiments, the amount of the compound and the amount of the checkpoint inhibitor are administered simultaneously, separately or sequentially.

In some embodiments, the checkpoint inhibitor is administered concurrently with, prior to, or after the PP2A inhibitor.

In some embodiments, the amount of checkpoint inhibitor and the amount of compound when administered together is more effective to treat the subject than when each agent at the same amount is administered alone.

In some embodiments, the amount of the compound and the amount of the checkpoint inhibitor when taken together is effective to reduce a clinical symptom of the cancer in the subject.

In some embodiments, the compound enhances the immunotherapeutic effect of the checkpoint inhibitor.

In some embodiments, the cancer is susceptible to treatment by an immune response.

In some embodiments, the immune checkpoint inhibitor is a CTLA-4 agent.

In some embodiments, the CTLA-4 checkpoint inhibitor is ipilimumab or tremelimumab.

In some embodiments, the immune checkpoint inhibitor is an Anti-PD-1 or Anti-PD-L1 agent.

In some embodiments, the PD-1 and/or PD-L1 checkpoint inhibitor is atezolizumab, nivolumab or pembrolizumab.

In some embodiments, the cancer is melanoma, renal cell carcinoma, prostate cancer, urothelial carcinoma or ovarian cancer.

In some embodiments, the cancer is melanoma.

In some embodiments, the PP2A inhibitor is administered at a dose of 0.25 mg/m², 0.5 mg/m², 0.83 mg/m², 1.25 mg/m², 1.75 mg/m², 2.33 mg/m², of 3.1 mg/m².

In some embodiments, the PP2A inhibitor is administered at a dose of 2.33 mg/m².

In some embodiments, the PP2A inhibitor is administered for 3 days every 3 weeks.

In some embodiments, the ipilimumab is administered intravenously at a dose of 0.5 mg/kg-10 mg/kg or less.

In some embodiments, the ipilimumab is administered intravenously over 90 minutes every 3 weeks or less.

In some embodiments, the atezolizunab is administered intravenously at a dose of 0.1 mg/kg-20 mg/kg or less.

In some embodiments, the atezolizumab is administered intravenously over 60 minutes every 3 weeks or less.

In some embodiments, the nivolumab is administered intravenously at a dose of 0.1 mg/kg-10 mg/kg or less.

In some embodiments, the nivolumab is administered intravenously over 60 minutes every 2 weeks or less.

In some embodiments, the pembrolizumab is administered intravenously at a dose of 1 mg/kg-10 mg/kg or less.

In some embodiments, the pembrolizumab is administered intravenously over 30 minutes every 3 weeks or less.

The present invention also provides a method of inhibiting the function of a CTLA-4 in T cells comprising administering to the T cells a PP2A inhibitor so as to thereby inhibit the function of the CTLA-4.

The present invention also provides a method of inhibiting the PD-1:PD-L1 interaction in T cells comprising administering to the T cells a PP2A inhibitor so as to thereby inhibit the interaction of PD-1:PD-L1.

In some embodiments, the method wherein the PP2A inhibitor has the structure:

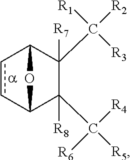

wherein
bond α is present or absent;
R₁ and R₂ together are ═O;
R₃ is OH, O—, OR₉, O(CH₂)₁₋₆R₉, SH, S—, or SR₉,
wherein R₉ is H, alkyl, alkenyl, alkynyl or aryl;
R₄ is

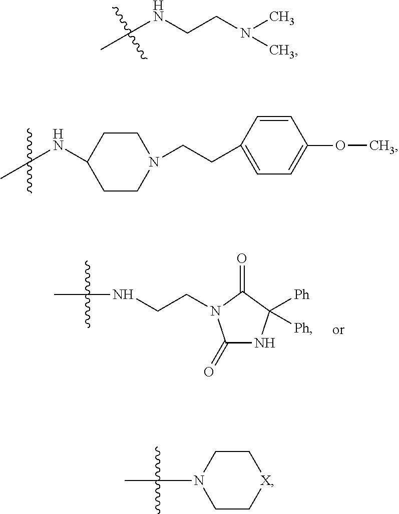

where X is O, S, NR₁₀, N⁺HR₁₀ or N⁺R₁₀R₁₀,
where each R₁₀ is independently H, alkyl, alkenyl, alkynyl, aryl,

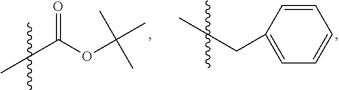

—CH₂CN, —CH₂CO₂R₁₁, or —CH₂COR₁₁,
wherein each R₁₁ is independently H, alkyl, alkenyl or alkynyl;
R₅ and R₆ taken together are ═O;
R₇ and R₈ are each H,
or a salt, zwitterion, or ester thereof.

In some embodiments, the compound has the structure:

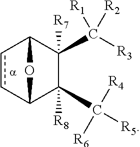

In some embodiments, bond α in the compound is present.

In some embodiments, bond α in the compound is absent.

In some embodiments, R₃ is OH, O—, or OR₉,
wherein R₉ is alkyl, alkenyl, alkynyl or aryl;
R₄ is

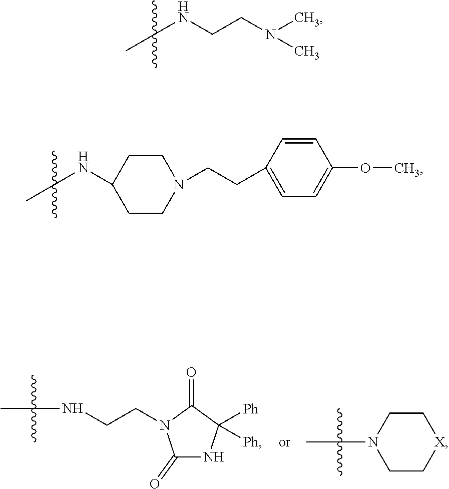

where X is O, S, NR₁₀, N⁺HR₁₀ or N⁺R₁₀R₁₀,
where each R₁₀ is independently H, alkyl, alkenyl, alkynyl, aryl,

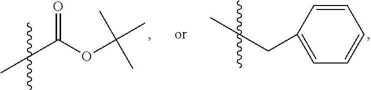

In some embodiments, R₃ is OH, O— or OR₉, where R₉ is H, methyl, ethyl or phenyl.

In some embodiments, R₃ is OH, O— or OR₉, wherein R₉ is methyl.

In some embodiments, R₄ is

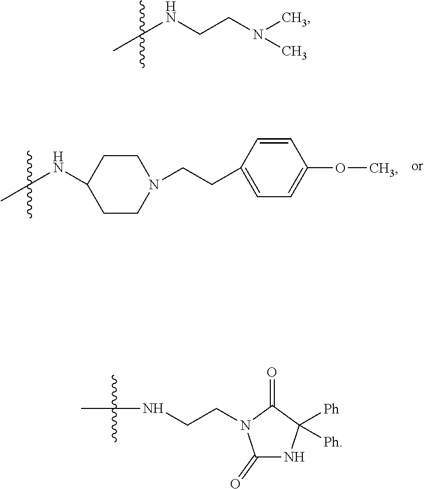

In some embodiments, R₄ is

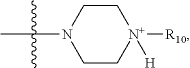

wherein R₁₀ is H, alkyl, alkenyl, alkynyl, aryl, or

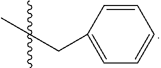 [SMILES: *Cc1ccccc1]

In some embodiments, R₄ is

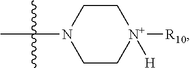

wherein R₁₀ is —H, —CH₃, —CH₂CH₃, or

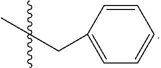 [SMILES: *Cc1ccccc1]

In some embodiments, R₄ is

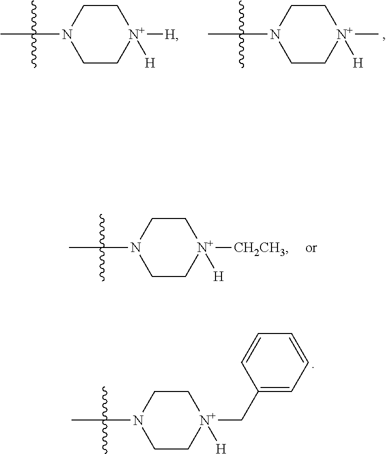

In some embodiments, R₄ is

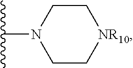

wherein R₁₀ is H, alkyl, alkenyl, alkynyl, aryl,

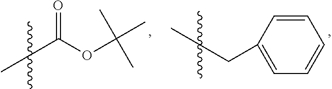

In some embodiments, R₄ is

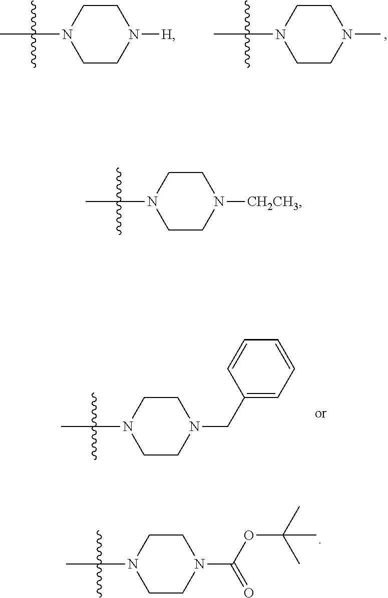

In some embodiments, R₄ is

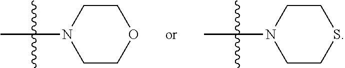 [SMILES: *N1CCOCC1 ; *N1CCSCC1]

In some embodiments, the compound has the structure

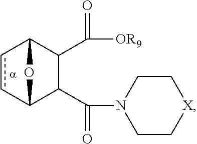

wherein
bond α is present or absent;
R₉ is present or absent and when present is H, alkyl, alkenyl, alkynyl or phenyl; and
X is O, NR₁₀, NH⁺R₁₀ or N⁺R₁₀R₁₀,
where each R₁₀ is independently H, alkyl, substituted alkyl, alkenyl, substituted alkenyl, alkynyl, substituted alkynyl, aryl,

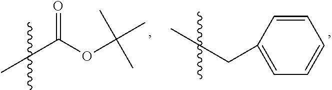

—CH₂CN, —CH₂CO₂R₁₂, or —CH₂COR₁₂,
where R₁₂ is H or alkyl,
or a salt, zwitterion or ester thereof.

In some embodiments, the compound has the structure

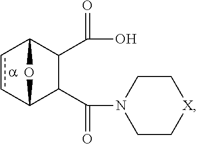

wherein
bond α is present or absent;
X is O or NR₁₀,
where each R₁₀ is independently H, alkyl, substituted alkyl, alkenyl, substituted alkenyl, alkynyl, substituted alkynyl, aryl,

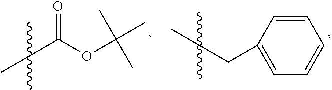

—CH₂CN, —CH₂CO₂R₁₂, or —CH₂COR₁₂,
where R₁₂ is H or alkyl,
or a salt, zwitterion or ester thereof.

In some embodiments, the compound has the structure

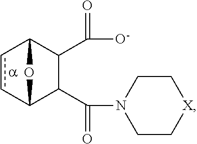

wherein
bond α is present or absent;
X is O or NH⁺R₁₀,
where R₁₀ is H, alkyl, substituted alkyl, alkenyl, substituted alkenyl, alkynyl, substituted alkynyl, aryl,

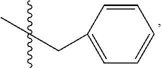 [SMILES: *Cc1ccccc1]

—CH₂CN, —CH₂CO₂R₁₂, or —CH₂COR₁₂,
where R₁₂ is H or alkyl,
or a salt, zwitterion or ester thereof.

In some embodiments, the compound has the structure

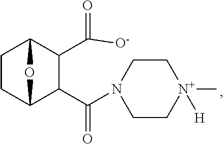 [SMILES: C[NH+]1CCN(C(=O)C2C(C(=O)[O-])[C@H]3CC[C@@H]2O3)CC1]

or a salt or ester thereof.

The present invention provides a method of inhibiting the function of CTLA-4 in T cells comprising administering to the T cells a PP2A inhibitor so as to thereby inhibit the function of CTLA-4.

The present invention also provides a method of inhibiting the function of CTLA-4 in a subject afflicted with cancer comprising administering to the subject a PP2A inhibitor so as to thereby inhibit the function of CTLA-4 in the subject.

The present invention further provides a method of increasing T-cell activation in a subject afflicted with cancer comprising administering to the subject a PP2A inhibitor so as to thereby increase the T-cell activation.

The present invention yet further provides a method of increasing T-cell response to cancers cells in a subject afflicted with cancer comprising administering to the subject a PP2A inhibitor so as to thereby increase the T-cell response to the cancers cells.

The present invention also provides a method of treating a subject afflicted with cancer comprising administering to the subject an effective amount of a CTLA-4 checkpoint inhibitor and an effective amount of a PP2A inhibitor, wherein the amounts when taken together are effective to treat the subject.

In some embodiments, the PP2A inhibitor alters the interaction of PP2A with CTLA-4.

In some embodiments, the PP2A inhibitor decreases the binding of PP2A to CTLA-4.

In some embodiments of any of the above methods, the cancer is susceptible to anti-CTLA-4 immunotherapy.

In some embodiments of any of the above methods, the subject has reduced T-cell activation mediated by CTLA-4.

The present invention also provides a method of treating a subject afflicted with cancer comprising administering to the subject an effective amount of a PP2A inhibitor so as to thereby treat the cancer, wherein the cancer is susceptible to anti-CTLA-4 immunotherapy.

The present invention also provides a method of treating a subject afflicted with cancer comprising administering to the subject an effective amount of a PP2A inhibitor so as to thereby treat the cancer, wherein the cancer is susceptible to immunotherapy.

The present invention also provides a method of treating a subject afflicted with cancer comprising administering to the subject an effective amount of a PP2A inhibitor so as to thereby treat the cancer, wherein the subject has reduced T cell activation mediated by CTLA-4.

In some embodiments of any of the above methods, the cancer is susceptible to anti-CTLA-4 immunotherapy.

In some embodiments of any of the above methods, the subject has reduced T-cell activation mediated by CTLA-4.

In some embodiments of any of the above methods, the cancer is melanoma, renal cell carcinoma, prostate cancer, urothelial carcinoma or ovarian cancer.

In some embodiments of any of the above methods, the cancer is melanoma.

In some embodiments of any of the above methods, the cancer susceptible to anti-CTLA-4 immunotherapy is melanoma.

In some embodiments, the cancer is pancreatic cancer.

In some embodiments, the cancer is pancreatic cancer, and the cancer cells of the pancreatic cancer overexpress Mad2.

In some embodiments, the cancer has abnormalities in PP2A function and/or in the DNA-damage-repair pathway.

In some embodiments, the subject is afflicted with fibrosarcoma, chondrosarcoma, thymoma, atypical carcinoid of lung, or ovarian, testicular, breast, or prostate cancer.

In some embodiments of the above method, the PP2A inhibitor is effective to treat a subject afflicted with a cancer.

In some embodiments, the above method further comprises administering an anti-cancer therapy concurrently with, prior to, or after the PP2A inhibitor.

In some embodiments, the anti-cancer therapy comprises administering a checkpoint inhibitor, for instance a CTLA-4 checkpoint inhibitor. In some embodiments of the above method, the PP2A inhibitor enhances the chemotherapeutic effect of the CTLA-4 checkpoint inhibitor.

In some embodiments of the above method, the CTLA-4 checkpoint inhibitor is an antibody.

In some embodiments of the above method, the PP2A inhibitor alters the interaction of PP2A with CTLA-4.

In some embodiments of the above method, the PP2A inhibitor increases the binding of PP2A to CTLA-4.

Cancers susceptible to anti-CTLA-4 immunotherapy include, but are not limited to, cancers which have been shown to be amenable to anti-CTLA-4 immunotherapy in pre-clinical or clinical trials.

Cancers susceptible to anti-PD-1 or anti-PD-L1 immunotherapy include, but are not limited to, cancers which have been shown to be amenable to anti-PD-1 or anti-PD-L1 immunotherapy in pre-clinical or clinical trials.

In some embodiments, the amount of the compound is effective to reduce a clinical symptom of the cancer in the subject.

In some embodiments, the treatment comprises increasing the amount of cytotoxic T cells in the subject.

In some embodiments, the treatment comprises increasing the amount of cytotoxic T cells that interact with cancer cells in the subject.

In some embodiments, the treatment comprises increasing the amount of cancer cells killed by cytotoxic T cells in the subject.

T cell types include “killer” cytotoxic CD8⁺ T cells and “helper” CD4⁺ T cells. The latter encompass subtypes involved in regulating immune responses, such as “T_{reg}” cells, and others that stimulate the acquired immune system, including recognition of “non-self” proteins that can stimulate killer T cells or antibody-producing B cells. Specific T cell clones, some of which are maintained after antigen exposure in low levels as “memory” T cells, are activated by particular MHC/epitope combinations, leading to cytokine release, clonal expansion, and acquired immune responses.

In some embodiments, the T cells are CD4+ T cells, CD8+ T cells, and/or CD4+CD8+ T cells.

In some embodiments, the cancer is hepatocellular carcinoma, human osteosarcoma, primary liver cancer, gastric cancer, ovarian cancer, endometrial cancer, colorectal cancer, non-small cell lung cancer, soft-tissue sarcoma, seminoma, breast cancer, lymphoma, fibrosarcoma, neuroblastoma, mucinous ovarian cancer, urothelial bladder cancer, squamous cell carcinoma of the uterine cervix, diffuse large B-cell lymphoma, lung adenoma, hepatoma, intestinal cancer, fibrosarcoma, osteosarcoma, prostate cancer, angiomyolipoma, mammary adenocarcinoma, acute myeloid leukemia, chronic lymphocytic leukemia, and multiple myeloma and other plasma cell neoplasms.

In some embodiments, the cancer is lung adenoma, hepatoma, hepatocellular carcinoma, intestinal cancer, lymphoma, fibrosarcoma, osteosarcoma, prostate cancer, angiomyolipoma, or mammary adenocarcinoma.

In some embodiments, the cancer is acute myeloid leukemia.

In some embodiments, the cancer is breast cancer, colon cancer, large cell lung cancer, adenocarcinoma of the lung, small cell lung cancer, stomach cancer, liver cancer, ovary adenocarcinoma, pancreas carcinoma, prostate carcinoma, promyelocytic leukemia, chronic myelogenous leukemia, acute lymphoblastic leukemia, chronic lymphocytic leukemia, multiple myeloma and plasma cell neoplasms, colorectal cancer, ovarian cancer, lymphoma, non-Hodgkin lymphoma, Hodgkin lymphoma, neuroblastoma, medulloblastoma, glioblastoma, chordoma, meningioma (non-malignant and malignant), diffuse intrinsic pontine glioma, or atypical teratoid/rhabdoid tumor.

In some embodiments of the above method, the cancer is a breast cancer, colon cancer, large cell lung cancer, adenocarcinoma of the lung, small cell lung cancer, stomach cancer, liver cancer, ovary adenocarcinoma, pancreas carcinoma, prostate carcinoma, acute promyelocytic leukemia, chronic myelogenous leukemia, acute lymphoblastic leukemia, chronic lymphocytic leukemia, multiple myeloma and plasma cell neoplasm, colorectal cancer, ovarian cancer, lymphoma, non-Hodgkin lymphoma or Hodgkin lymphoma.

In some embodiments of the above method, the cancer is a brain cancer.

In some embodiments of the above method, the brain cancer is a glioma, pilocytic astrocytoma, low-grade diffuse astrocytoma, anaplastic astrocytoma, glioblastoma multiforme, oligodendrogliorna, ependymoma, meningioma, pituitary gland tumor, primary central nervous system lymphoma, medulloblastoma, craniopharyngioma, or diffuse intrinsic pontine glioma.

In some embodiments of the above method, further comprising administering to the subject an anti-cancer agent.

In some embodiments of the above method, the anti-cancer agent is selected from x-radiation or ionizing radiation.

In some embodiments of the above method, the target cell is a cancer cell.

In some embodiments of the above method, the cancer cell is a breast cancer, colon cancer, large cell lung cancer, adenocarcinoma of the lung, small cell lung cancer, stomach cancer, liver cancer, ovary adenocarcinoma, pancreas carcinoma, prostate carcinoma, promyelocytic leukemia, chronic myelogenous leukemia, acute lymphoblastic leukemia, colorectal cancer, ovarian cancer, lymphoma, non-Hodgkin lymphoma or Hodgkin lymphoma cell.

Analogs of LB-100 have analogous activity to LB-100 and exhibit similar effects in the methods described herein. Such analogs include the compounds described in PCT International Application Publication No. WO 2008/097561, published Aug. 14, 2008; PCT International Application Publication No. WO 2010/014254, published Feb. 4, 2010; PCT International Application Publication No. WO 2015/073802, published May 21, 2015; and PCT International Application Publication No. WO 2016/186963, published Nov. 24, 2016, the contents of each of which are hereby incorporated by reference.

Compounds which act as prodrugs for the in vivo delivery of LB-100 and/or endothal have analogous activity to LB-100 and exhibit similar effects in the methods described herein. More specifically, administration of the prodrug provides a similar effect to the administration of LB-100. Pro-drugs of LB-100 and/or endothal include the compounds described in PCT International Application Publication No. WO 2015/073802, published May 21, 2015; and PCT International Application Publication No. WO 2016/186963, published Nov. 24, 2016, the contents of each of which are hereby incorporated by reference.

Except where otherwise specified, when the structure of a compound used in the method of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds in the method disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein. It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein. Isotopically-labeled compounds can generally be prepared by conventional techniques known to those skilled in the art using appropriate isotopically-labeled reagents in place of the non-labeled reagents employed.

In some embodiments, the method wherein the subject is administered a pharmaceutical composition comprising a compound of the present invention and at least one pharmaceutically acceptable carrier for treating the cancer in the subject.

In some embodiments, the pharmaceutical composition wherein the pharmaceutically acceptable carrier comprises a liposome.

In some embodiments, the pharmaceutical composition wherein the compound is contained in a liposome or microsphere.

In some embodiments, the pharmaceutical composition comprises the PP2A inhibitor and the CTLA-4 checkpoint inhibitor.

In some embodiments of any of the above methods or uses, the subject is a human.

In some embodiments of any of the above methods or uses, the compound and/or the CTLA-4 checkpoint inhibitor is orally administered to the subject.

The present invention provides a PP2A inhibitor for use in inhibiting the function of CTLA-4 in T cells.

The present invention provides a PP2A inhibitor for use in inhibiting the function of CTLA-4 in a subject afflicted with cancer.

The present invention provides a PP2A inhibitor for use in increasing T cell activation in a subject afflicted with cancer.

The present invention provides a PP2A inhibitor for use in increasing T cell response to cancers cells in a subject afflicted with cancer.

The present invention provides a PP2A inhibitor for use in treating a subject afflicted with cancer, wherein the cancer is susceptible to anti-CTLA-4 immunotherapy.

The present invention provides a PP2A inhibitor for use in treating a subject afflicted with cancer, wherein the subject has reduced T cell activation mediated by CTLA-4.

The present invention provides a PP2A inhibitor in combination with a CTLA-4 checkpoint inhibitor for use in treating a subject afflicted with cancer.

Use of a PP2A inhibitor for inhibiting the function of CTLA-4 in T cells.

Use of a PP2A inhibitor for inhibiting the function of CTLA-4 in a subject afflicted with cancer.

Use of a PP2A inhibitor for increasing T cell activation in a subject afflicted with cancer.

Use of a PP2A inhibitor for increasing T-cell response to cancers cells in a subject afflicted with cancer.

Use of a PP2A inhibitor for treating a subject afflicted with cancer, wherein the cancer is susceptible to anti-CTLA-4 immunotherapy.

Use of a PP2A inhibitor for treating a subject afflicted with cancer, wherein the subject has reduced T-cell activation mediated by CTLA-4.

Use of a PP2A inhibitor in combination with a CTLA-4 checkpoint inhibitor for treating a subject afflicted with cancer.

The present invention also provides a method of optimizing the concentration of LB-100 in the bloodstream of a subject who has been administered a dosage of LB1-00 comprising:
(a) measuring the plasma concentration of LB-100 in the subject;
(b) determining whether a further LB-100 dose needs to be administered to the subject based on whether the measurement in (a); and
(c) administering a further dosage or dosages of the LB-100 as necessary based on the determination in (b).

In some embodiments, the above step (b) comprises determining whether a further LB-100 dose needs to be administered to the subject based on whether the measurement in (a) is above, below or equal to the Minimum Effective Concentration (MEC) of LB-100.

In some embodiments, the initial dose of LB-100 administered to the subject is an amount of from 0.1 mg/m² to 5 mg/m².

In some embodiments, the further dose of LB-100 administered to the subject is an amount of from 0.1 mg/m² to 5 mg/m².

In some embodiments, the compound is administered at a dose of 0.25 mg/m², 0.5 mg/m², 0.83 mg/m², 1.25 mg/m², 1.75 mg/m², 2.33 mg/m², or 3.1 mg/m².

In some embodiments, the compound is administered at a dose of 2.33 mg/m².

In some embodiments, the compound is administered for 3 days every 3 weeks.

In some embodiments, the further dose of LB-100 administered to the subject is an amount 25% less than the initial dose.

In some embodiments, the further dose of LB-100 administered to the subject is an amount 50% less than the initial dose.

In some embodiments, the further dose of LB-100 administered to the subject is an amount 75% less than the initial dose.

In some embodiments, the further dose of LB-100 administered to the subject is an amount 25% more than the initial dose.

In some embodiments, the further dose of LB-100 administered to the subject is an amount 50% more than the initial dose.

In some embodiments, the further dose of LB-100 administered to the subject is an amount 75% more than the initial dose.

In some embodiments, the subject is further treated with an anti-cancer therapy concurrently with, prior to, or after the administering.

Examples of anti-cancer therapy include radiation therapy or chemotherapy, targeted therapy to promote antigen release, vaccination to promote antigen presentation, agonist for co-stimulatory molecules or blockade of co-inhibitory molecules to amplify T-cell activation, trafficking inhibition of regulatory T cells or myeloid-derived suppressor cells, anti-vascular endothelial growth factor to stimulate intratumoral T-cell infiltration, adoptive cell transfer to increase cancer recognition by T-cell infiltration, or stimulate tumor killing. Further examples may be found in Swart et al. 2016; Topalian et al. 2015; and Tsiatas et al. 2016.

In some embodiments, the anti-cancer therapy comprises immunotherapy. The term “immunotherapy” refers to the treatment of a subject afflicted with a disease by a method comprising inducing, enhancing, suppressing or otherwise modifying an immune response. Immunotherapy agents may include antibody agents targeting one or more of CTLA-4, PD-1, PD-L1, GITR, OC40, LAG-3, KIR, TIM-3, B7-H3, B7-H4, CD28, CD40, and CD137.

In some embodiments, the anti-cancer therapy comprises administering an anti-cancer agent.

In some embodiments, the anti-cancer agent is an immune checkpoint modulator. The term “immune checkpoint modulator” refers to an agent that interacts directly or indirectly with an immune checkpoint. Immune checkpoint modulators may be administered to overcome inhibitory signals and permit and/or augment an immune attach against cancer cells. In some embodiments, an immune checkpoint modulator increases an immune effector response (e.g. cytotoxic T cell response). In some embodiments, an immune checkpoint modulator reduces, removes, or prevents immune tolerance to one or more antigens. For example, immune checkpoint modulators may facilitate immune cell responses by decreasing, inhibiting, or abrogating signaling by negative immune response regulators (e.g. CTLA4), by stimulating or enhancing signaling of positive regulators of immune response (e.g. CD28), or by preventing autoimmune responses and limiting immune cell-mediated tissue damage.

In some embodiments, the anti-cancer agent comprises an antibody or an antigen-binding portion thereof.

In some embodiments, the anti-cancer agent comprises a Programmed Death-Ligand 1 (PD-L1) inhibitor. In some embodiments, the PD-L1 inhibitor is atezolizumab.

Atezolizumab, the active ingredient of Tecentriq™, is a human programmed death ligand-1 (PD-L1) blocking antibody. Atezolizumab is identified by specific antibodies (Tecentriq, Food and Drug Administration Approved Labeling (Reference ID:4000525) [online], Genentech Inc., 2016 [retrieved on Feb. 24, 2017], Retrieved from the Internet: <URL: www.accessdata.fda.gov/drugsatfda_docs/label/2016/7610411bl.pdf>).

The recommended dose and schedule for atezolizumab is 1200 mg administered intravenously over 60 minutes every 3 weeks until disease progression or unacceptable toxicity. Subsequent infusions may be delivered over 30 minutes if the first infusion is tolerated.

In some embodiments, the administration of atezolizumab comprises 1 mg/kg, 2 mg/kg, 3 mg/kg, 4 mg/kg, 5 mg/kg, 10 mg/kg, 15 mg/kg, 20 mg/kg or less of atezolizumab.

In some embodiments, the periodic administration of atezolizumab comprises 1, 2, 3, 4 or less administrations of atezolizumab.

In some embodiments, the administration of nivolumab is every 2 or 3 weeks or less.

In some embodiments, the antibody or antigen-binding portion thereof binds specifically to a Programmed Death-1 (PD-1) receptor and inhibits PD-1 activity (“anti-PD-1 antibody”). In some embodiments, the anti-PD-1 antibody is nivolumab or pembrolizumab.

Nivolumab, the active ingredient of Opdivo™, is a human Programmed Death receptor-1 (PD-1) blocking antibody. Nivolumab is identified by specific antibodies (Opdivo™, Food and Drug Administration Approved Labeling (Reference ID:3677021) [online], Bristol-Myers Squibb, 2014 [retrieved on Feb. 24, 2017], Retrieved from the Internet: <URL: www.accessdata.fda.gov/drugsatfda_docs/label/2014/1255541bl.pdf>).

The recommended dose and schedule for nivolumab is 3 mg/kg administered intravenously over 60 minutes every 2 weeks for 4 doses until disease progression or unacceptable toxicity.

In some embodiments, the administration of nivolumab comprises 0.1 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg or less of nivolumab.

In some embodiments, the periodic administration of nivolumab comprises 1, 2, 3, 4 or less administrations of nivolumab.

In some embodiments, the administration of nivolumab is every 2 or 3 weeks or less.

Pembrolizumab, the active ingredient of Keytruda™, is a human programmed death receptor-1 (PD-1) blocking antibody. Pembrolizumab is identified by specific antibodies (Keytruda, Food and Drug Administration Approved Labeling (Reference ID:3621876) [online], Merck & Co., 2014 [retrieved on Feb. 24, 2017], Retrieved from the Internet: <URL: www.accessdata.fda.gov/drugsatfda_docs/label/2014/1255141bl.pdf>).

The recommended dose and schedule for pembrolizumab is 2 mg/kg administered intravenously over 30 minutes every 3 weeks until disease progression or unacceptable toxicity.

In some embodiments, the administration of pembrolizumab comprises 1 mg/kg, 2 mg/kg, 3 mg/kg, 4 mg/kg, 5 mg/kg, 6 mg/kg, 7 mg/kg, 8 mg/kg, 9 mg/kg, 10 mg/kg or less of pembrolizumab.

In some embodiments, the periodic administration of pembrolizumab comprises 1, 2, 3, 4, 5, 6, 7, 8, 9, 10, 11, 12, 13, 14, 15, 16, 17, 18 or less administrations of pembrolizumab.

In some embodiments, the administration of pembrolizumab is every 2 or 3 weeks or less.

In some embodiments the antibody or antigen-binding portion thereof binds specifically to Cytotoxic T-Lymphocyte Antigen-4 (CTLA-4) and inhibits CTLA-4) activity (“anti-CTLA-4 antibody”). In another embodiment. In some embodiments, the anti-CTLA-4 antibody is ipilimumab or tremelimumab.

Ipilimumab, the active ingredient of Yervoy™, is a human cytotoxic T-lymphocyte antigen 4 (CTLA-4)-blocking antibody. Ipilimumab is identified by specific antibodies (Yervoy, Food and Drug Administration Approved Labeling (Reference ID: 3839653) [online], Bristol-Myers Squibb, 2015 [retrieved on Feb. 24, 2017], Retrieved from the Internet: <URL: www.accessdata.fda.gov/drugsatfda_docs/label/2015/125377s0731bl.pdf>).

The recommended dose and schedule for ipilimumab for unresectable or metastatic melanoma is 3 mg/kg administered intravenously over 90 minutes every 3 weeks for 4 doses. The recommended dose and schedule for ipilimumab for adjuvant treatment of melanoma is 10 mg/kg administered intravenously over 90 minutes every 3 weeks for 4 doses followed by 10 mg/kg every 12 weeks for up to 3 years.

In some embodiments, the administration of ipilimumab comprises 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg or less of ipilimumab.

In some embodiments, the periodic administration of ipilimumab comprises 1, 2, 3, 4, 5, 6, 7, 8, 9, 10, 11, 12, 13, 14, 15, 16 or less administrations of ipilimumab.

The present invention also provides a method of treating a tumor or cancer in a subject comprising administering to the subject an effective amount of a PP2A inhibitor, wherein the tumor or cancer is susceptible to treatment by an immune response.

The present invention also provides a method of increasing a T-cell response to cancer cells in a subject afflicted with cancer comprising administering to the subject an amount of a PP2A inhibitor effective to increase the T-cell response.

In some embodiments, the PP2A inhibitor has the structure:

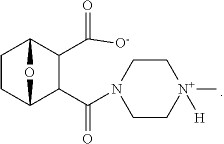 [SMILES: C[NH+]1CCN(C(=O)C2C(C(=O)[O-])[C@H]3CC[C@@H]2O3)CC1]

In some embodiments, the method further comprising administering one or more additional anti-cancer agent.

The present invention also provides a method of treating a subject afflicted with cancer comprising administering to the subject an effective amount of a PP2A inhibitor in combination with an effective amount of an anti-cancer therapy, wherein the amounts when taken together are effective to treat the subject.

The present invention also provides a method of treating a subject afflicted with cancer and receiving anti-cancer therapy comprising administering to the subject an effective amount of PP2A inhibitor effective to enhance treatment relative to the anti-cancer therapy alone.

In some embodiments, the cancer is susceptible to treatment by an immune response.

The compounds used in the method of the present invention are protein phosphatase 2A (PP2A) inhibitors. Methods of preparation may be found in Lu et al., 2009; U.S. Pat. No. 7,998,957 B2; and U.S. Pat. No. 8,426,444 B2. Compound LB-100 is an inhibitor of PP2A in vitro in human cancer cells and in xenografts of human tumor cells in mice when given parenterally in mice. LB-100 inhibits the growth of cancer cells in mouse model systems.

As used herein, a “symptom” associated with reperfusion injury includes any clinical or laboratory manifestation associated with reperfusion injury and is not limited to what the subject can feel or observe.

As used herein, “treatment of the diseases” or “treating”, e.g. of reperfusion injury, encompasses inducing prevention, inhibition, regression, or stasis of the disease or a symptom or condition associated with the disease.

As used herein, “inhibition” of disease progression or disease complication in a subject means preventing or reducing the disease progression and/or disease complication in the subject.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n-1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₂₀ alkyl, C₂-C₂₀ alkyl, C₃-C₂₀ alkyl, C₄-C₂₀ alkyl and so on. An embodiment can be C₁-C₃₀ alkyl, C₂-C₃₀alkyl, C₃-C₃₀ alkyl, C₄-C₃₀ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n-1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₂-C₂₀ alkenyl, C₃-C₂₀ alkenyl, C₂-C₃₀ alkenyl, or C₃-C₃₀ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n-1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₁₂ alkynyl, C₂-C₂₀ alkynyl, C₃-C₂₀ alkynyl, C₂-C₃₀ alkynyl, or C₃-C₃₀ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynyl, or aryl groups as hereinabove defined.

Each occurrence of alkyl, alkenyl, or alkynyl is branched or unbranched, unsubstituted or substituted.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprolactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrrolidone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprolactone.

Oral delivery systems include tablets and capsules. These can contain excipients such as binders (e.g., hydroxypropylmethylcellulose, polyvinyl pyrrolidone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphtholate, mesylate, glucoheptonate, lactobionate, and laurylsulphonate salts and the like. (See, e.g., Berge et al. (1977) “Pharmaceutical Salts”, J. Pharm. Sci. 66:1-19).

The present invention includes esters or pharmaceutically acceptable esters of the compounds of the present method. The term “ester” includes, but is not limited to, a compound containing the R—CO—OR′ group. The “R—CO—O” portion may be derived from the parent compound of the present invention. The “R′” portion includes, but is not limited to, alkyl, alkenyl, alkynyl, heteroalkyl, aryl, and carboxy alkyl groups.

The present invention includes pharmaceutically acceptable prodrug esters of the compounds of the present method. Pharmaceutically acceptable prodrug esters of the compounds of the present invention are ester derivatives which are convertible by solvolysis or under physiological conditions to the free carboxylic acids of the parent compound. An example of a pro-drug is an alkyl ester which is cleaved in vivo to yield the compound of interest.

The compound, or salt, zwitterion, or ester thereof, is optionally provided in a pharmaceutically acceptable composition including the appropriate pharmaceutically acceptable carriers.

As used herein, an “amount” or “dose” of an agent measured in milligrams refers to the milligrams of agent present in a drug product, regardless of the form of the drug product.

The National Institutes of Health (NIH) provides a table of Equivalent Surface Area Dosage Conversion Factors below (Table A) which provides conversion factors that account for surface area to weight ratios between species.

TABLE A
Equivalent Surface Area Dosage Conversion Factors

| | To | | | | | |
|---|---|---|---|---|---|---|
| | | Mouse | Rat | Monkey | Dog | Man |
| | | 20 g | 150 g | 3 kg | 8 kg | 60 kg |
| From | Mouse | 1 | ½ | ¼ | ⅙ | 1/12 |
| | Rat | 2 | 1 | ½ | ¼ | 1/7 |
| | Monkey | 4 | 2 | 1 | ⅗ | ⅓ |
| | Dog | 6 | 4 | 1⅔ | 1 | ½ |
| | Man | 12 | 7 | 3 | 2 | 1 |

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to-one percent of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

For the foregoing embodiments, each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Combination Therapy

The administration of two drugs to treat a given condition, such as melanoma, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug. (Guidance for Industry, 1999) Thus, when two drugs are administered to treat the same condition, it is unpredictable whether each will complement, have no effect on, or interfere with the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side effect profile of each drug.

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs. (Guidance for Industry, 1999)

### EXPERIMENTAL DETAILS

### Example 1. PP2A Inhibition and CTLA-4 Activity

Compound LB-100 and other homologs of LB-100 disclosed herein inhibit the function of CTLA-4 in T cells by altering the interaction of CTLA-4 and PP2A, thereby blocking CTLA-4 mediated inhibition of T-cell activation. Such interaction results in increased T-cell activation.

### Example 2. In Vitro Studies: PP2A and CTLA4

Primary human T cells and Jurkat T cells are treated with LB-100 and T-cell activation levels measured. LB-100 increases activation of the T cells.

Primary human T cells and Jurkat T cells are treated with LB-100 and PP2A:CTLA-4 interaction is assessed. LB-100 decreases the interaction of PP2A and CTLA-4.

Primary human T cells and Jurkat T cells are treated with LB-100 and levels of PP2A phosphorylation are measured. LB-100 increases phosphorylation in the T cells.

### Example 3. Administration of LB-100 and Analogs

An amount of compound LB-100 is administered to a subject afflicted with cancer. The amount of the compound is effective to treat the cancer by increasing the number of cytotoxic T cells in the subject.

An analog of compound LB-100 disclosed herein is administered to a subject afflicted with cancer. The amount of the compound is effective to treat the cancer by increasing the number of cytotoxic T cells in the subject.

An amount of compound LB-100 is administered to a subject afflicted with melanoma. The amount of the compound is effective to treat the cancer by increasing the number of cytotoxic T cells in the subject.

An analog of compound LB-100 disclosed herein is administered to a subject afflicted with melanoma. The amount of the compound is effective to treat the cancer by increasing the number of cytotoxic T cells in the subject.

### Example 4. Administration of LB-100 in Combination with a CTLA-4 Checkpoint Inhibitor

An amount of compound LB-100 in combination with a CTLA-4 checkpoint inhibitor is administered to a subject afflicted with cancer. The amount of the compound and inhibitor is effective to treat the subject.

An amount of compound LB-100 in combination with a CTLA-4 checkpoint inhibitor is administered to a subject afflicted with cancer. The amount of the compound is effective to enhance the anti-cancer activity of the CTLA-4 checkpoint inhibitor.

An amount of compound LB-100 in combination with a CTLA-4 checkpoint inhibitor is administered to a subject afflicted with melanoma. The amount of the compound and inhibitor is effective to treat the subject.

An amount of compound LB-100 in combination with a CTLA-4 checkpoint inhibitor is administered to a subject afflicted with melanoma. The amount of the compound is effective to enhance the anti-cancer activity of the CTLA-4 checkpoint inhibitor.

### Example 5: Assessment of Efficacy of LB-100 as Add-on Therapy to Ipilimumab or Tremelimumab

The add-on therapy provides a synergistic effect, and allows for lower doses with reduced side effects.

Periodic administration of LB-100 as an add-on therapy for a human patient afflicted with melanoma who is already receiving Ipilimumab or Tremelimumab provides a clinically meaningful advantage and is more effective (provides at least an additive effect or more than an additive effect) in treating the patient than when ipilimumab or tremelimumab is administered alone (at the same dose).

Periodic administration Ipilimumab or Tremelimumab as an add-on therapy for a human patient afflicted with melanoma who is already receiving of LB-100 provides a clinically meaningful advantage and is more effective (provides at least an additive effect or more than an additive effect) in treating the patient than when LB-100 is administered alone (at the same dose).

The add-on therapies also provides efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in increasing the amount of cytotoxic T cells in patients with melanoma;
2. The add-on therapy is more effective (provides an additive effect or more than an additive effect) in slowing the progression of melanoma in patients with melanoma; and/or
3. The add-on therapy is more effective (provides an additive effect or more than an additive effect) in reducing the function of CTLA-4 in the melanoma patients.

### Discussion (Examples 1-5)

The importance of the immune system in the context of cancer has increasingly been recognized with the development of cancer immunotherapy. The natural control mechanism of the immune system to prevent autoimmunity is often co-opted by tumors to evade immunosurveillance. Checkpoint molecules, such as programmed death-1 (PD-1) and cytotoxic T lymphocyte-associated protein 4 (CTLA-4), are negative regulators of the immune system that are constitutively activated by tumors to suppress activity of cytotoxic T cells that recognize neo-antigens of tumors (Topalian et al. 2015). Immunosuppressive regulatory T cells (Tregs) are also recruited to the tumor microenvironment (TME) to reduce effectiveness of the CD8 T cells. Monoclonal antibodies blocking PD-1 or CTLA-4 signaling could induce durable long-term responses in some patients with metastatic melanoma. This led to the approval of Ipilimumab (anti-CTLA-4) in 2011 and nivolumab (anti-PD-1) in 2014 by the U.S. Food and Drug Administration for treatment of advanced melanoma. Current clinical trials are underway to expand the use of checkpoint inhibitors to multiple other cancers, ushering a paradigm shift in the approach to cancer therapy. However, only a subset of patients responds to checkpoint inhibition effectively as single agent, highlighting the fact that multiple redundant mechanisms are involved in creating an immunosuppressive TME. Therefore, an active area of research is to identify combination strategies that could augment the effect of checkpoint inhibition.

Cytotoxic T lymphocyte associated antigen-4 (CTLA-4) is an activation-induced glycoprotein of the Immunoglobulin superfamily, whose primary function is to down-regulate T cell responses (Brunet, J. F. et al. 1987). CTLA-4 is expressed on the surface of T cells, where it primarily suppresses their early stages of activation by inducing inhibitory downstream T-cell receptor (TCR) signaling and counteracting activity of the T-cell costimulatory receptor, CD28, thus inhibiting T-cell activation and increasing immune tolerance to certain diseases, e.g. cancer. Several mechanisms, including antagonism of CD28-dependent costimulation and direct negative signaling have been documented to explain the inhibitory capacity of CTLA-4 (Carreno, B. M. et al. 2000). Since the cytoplasmic tail of CTLA-4 lacks intrinsic enzymatic activity, the delivery of such a negative signal is likely provided through the association of CTLA-4 with key signaling molecules (Teft, W. A. Et al. 2006).

Inhibition of CTLA-4 has been targeted for the treatment of cancers by way of an immune-checkpoint blockade. Cellular and murine malignancy models demonstrate that blockade of cytotoxic T lymphocyte antigen-4 (CTLA-4), a negative regulator of T cell responses, augments endogenous responses to tumor cells, thus leading to tumor cell death when utilized on its own or with other therapeutic interventions (Grosso, J. F. et al. 2013). Preclinical findings have translated into clinical development of a fully human, IgG1 monoclonal antibody (mAb), ipilimumab and a fully human, IgG2 mAb, tremelimumab, each of which bind CTLA-4.

Phosphorylation of proteins by kinases and their dephosphorylation by phosphatases are critical components of cellular signaling pathways regulating a multiplicity of processes including cell proliferation and cell death (Shi et al. 2009). Although phosphatases have long been considered potentially important targets for cancer treatment, there has been little effort to develop phosphatase inhibitors due to concern over toxicity (Janssens et al. 2012).

Protein phosphatase 2A (PP2A) is a ubiquitous serine/threonine phosphatase that dephosphorylates numerous proteins of both ATM/ATR-dependent and -independent response pathways (Mumby M. 2007). PP2A is implicated in a diverse set of cellular processes. In the immune system, PP2A has been shown to associate with CTLA-4 and mediates dephosphorylation of Akt leading to inhibition of activated T cells (Parry et al. 2005). Pharmacologic inhibition of PP2A has previously been shown to sensitize cancer cells to radiation-mediated DNA damage via constitutive phosphorylation of various signaling proteins, such as p53, γH2AX, PLK1 and Akt, resulting in cell cycle deregulation, inhibition of DNA repair, and apoptosis (Wei, D. et al. 2013).

Cantharidin, the principle active ingredient of blister beetle extract (Mylabris), is a compound derived from traditional Chinese medicine that has been shown to be a potent inhibitor of PP2A (Efferth, T. et al. 2005). Although cantharidin has previously been used in the treatment of hepatomas and has shown efficacy against multidrug-resistant leukemia cell lines (Efferth, T. et al. 2002), its severe toxicity limits its clinical usefulness. Cantharidin, a naturally occurring toxin, and its demethylated analog, norcantharidin, both potent inhibitors of PP2A (Bertini et al. 2009) were reported to have anti-cancer activity in patients in China with gastrointestinal cancers (Wang et al. 1989) although little clinical detail is available.

Fostriecin, another selective inhibitor of PP2A was evaluated in several US NCI-sponsored phase 1 trials over twenty years ago. In the largest trial, fostriecin was associated with disease stability in 16 (34.8%) of 46 solid tumor patients without dose-limiting toxicity (DLT) (Lê et al. 2004). No trials were completed because of insufficient drug supply.

LB-100 is a small molecule derivative of cantharidin with significantly less toxicity. LB-100 and its lipid-soluble homolog, LB-102, inhibit proliferation of cell lines from a variety of human solid tumors. Both compounds potentiate the activity without significantly increasing the toxicity of cisplatin, doxorubicin, and temozolomide against xenografts of pancreatic and hepatocellular carcinoma; fibrosarcoma; pheochromocytoma; neuroblastoma; and glioblastoma and of focal X-ray against pancreatic, nasopharyngeal and glioblastoma xenografts (Bai et al., 2014a; Bai et al., 2014b; Zhang et al., 2010; Matiniova et al., 2011; Lu et al., 2009; Wei et al., 2013; Lv et al., 2014; Gordon et al., 2015). In addition, LB-100 reversed resistance to cisplatin in ovarian carcinoma and medulloblastoma xenografts (Chang et al., 2015; Ho et al., 2016). Previous pre-clinical studies have shown that LB-100 can enhance the cytotoxic effects of temozolomide, doxorubicin, and radiation therapy against glioblastoma (GBM), metastatic pheochromocytoma, and pancreatic cancer (Wei, D. et al. 2013; Lu, J. et al. 2009; Zhang, C. et al. 2010; Martiniova, L. et al. 2011).

LB-100 is also undergoing a Phase 1 study in combination with docetaxel for the treatment of solid tumors (Chung, V. 2013). LB-100 is a novel, first-in-class, small molecule inhibitor of protein phosphatase 2A (PP2A) recently shown in a Phase 1 trial to be well-tolerated at doses associated with stabilization of progressive solid tumors (Chung et al. 2017). PP2A has been implicated in mediating Akt signaling downstream of CTLA-4 (Parry et al. 2005). In an in vivo pooled short hairpin RNA screen, Ppp2r2d, a regulatory subunit of PP2A, when knocked down was found to increase proliferation of tumor infiltrating lymphocytes to the greatest extent among all genes in the RNA library and identified PP2A as a key regulator in suppressing T-cell proliferation in the tumor microenvironment (Zhou et al. 2014). In addition, PP2A was found to be essential for regulatory-T-cell (Treg) function (Apostolidis et al. 2014).

While multiple pre-clinical studies have shown LB-100 to be an effective chemo- or radio-sensitizer in various tumor models (Hone et al. 2015), none have studied its effect on the immune system.

Impairment of CTLA-4 function allows cancer patients to mount a more effective cytotoxic t-cell attack on their cancers. Unlike the leading clinically used modulator of CTLA-4, the antibody ipilimumab (Yervoy), which is associated with significant toxicity at therapeutically effective doses, compound LB-100 is associated with stability of several different types of cancer in the absence of any significant toxicity or side effects.

There are a number of reports of clinical activity by inhibiting CTLA-4 with ipilimumab, particularly in patients with advanced melanoma. These studies show that single-agent ipilimumab and in combination with a cytotoxic drug such as dacarbazine or with another immune checkpoint inhibitor, nivolumab, cause regression of cancers. Alone or in combination with other agents, ipilimumab is associated with significant toxicity such as in the study by Hodi et al. (2010) in which up to 15% of patients had grade 3 or 4 adverse events including a 2.1% incidence of death. Wolchok et al (2013) studied ipilimumab plus nivolumab in patients with advanced melanoma, but grade 3 or 4 adverse events occurred in 53% of patients. Thus, the availability of a molecule such as LB-100 which has no limiting toxicity at doses associated with stabilization of progressive cancer and has anti CTLA-4 activity is an attractive clinical candidate for the treatment of human cancers. At present, there are no molecular determinants of clinical response to CTLA-4 modulation. Snyder et al (2014) reported a genetic basis for potential benefit from CTLA-4 treatment of melanoma, providing a potential basis for characterizing candidate tumor neoantigens for each patient.

Without being bound by any theory, it is believed that CTLA-4 interacts with PP2A in human T cells and such interaction is essential for the proper function of CTLA-4. When functioning correctly, CTLA-4 inhibits T-cell activation, thus reducing the immunoresponse to cancer cells. Administration of a PP2A inhibitor to a subject with cancer alters the interaction of PP2A with CTLA-4, thereby disrupting the normal function of CTLA-4. The reduction or elimination of CTLA-4 function in T cells leads to increased T-cell activation. Increased T-cell activation results in an increase in cytotoxic T cells in the subject which target and destroy the cancer cells. As with the existing immune checkpoint inhibitors, LB-100 is effective alone and/or in combination with other checkpoint inhibitors.

### Example 6. Assessment of LB-100 in Adult Patients with Progressive Solid Tumors

PP2A has long been recognized as a potentially important target for cancer therapy because of its regulatory role in cell division, DNA-damage-response, homologous recombination repair, and mitotic exit but inhibition of this enzyme has been considered likely to be too toxic for clinical use. This study shows the safety, tolerability, and potential anti-cancer activity of an inhibitor of PP2A, LB-100, in patients with refractory solid tumors. PP2A activity is altered by mutation directly or indirectly in many types of cancer. The availability of a clinically safe inhibitor of PP2A opens a promising new avenue for cancer therapy, namely pharmacologic inhibition of PP2A in cancers with mutationally acquired abnormalities in PP2A function and/or in the DNA-damage-repair pathway. The results of this study support further development of LB-100 alone and in combination with other agents for the treatment of cancers.

Purpose: To assess the safety, tolerability, and potential activity of LB-100, a first-in-class small molecule inhibitor of protein phosphatase 2A (PP2A), in adult patients with progressive solid tumors.

Experimental Design: LB-100 was administered intravenously daily for 3 days in 21-day cycles in a 3+3 dose-escalation design. The primary goal was to determine the maximum tolerated dose and recommended phase 2 dose (ClinicalTrials.gov: NCT01837667).

### Materials and Methods

Eligible patients were aged 18 years or older with proven progressive solid tumors who had failed standard treatments. Patients had to have a life expectancy of at least 12 weeks, an ECOG performance status of 0 or 1, and be able to give informed consent. Before participation, patients must have recovered to baseline or less than grade 1 toxicity from prior treatments, have adequate bone marrow (an absolute neutrophil count >1.5×10⁹/L and platelet count >100×10⁹/L); kidney (serum creatinine <1.2 mg/dL and if >1.2 mg/dL, creatinine clearance [Cockcroft-Gault method]>60 mL/min/1.73 m²; and hepatic function (plasma total bilirubin <1.5 mg/dL, alanine transaminase (ALT) and aspartate transaminase (AST)<2.5× upper limit of normal). They must not have any other uncontrolled systemic disease. Women of childbearing potential had to have a negative serum or urine pregnancy test result.

### Study Design and Treatment

An open-label, dose-escalation, phase I study was performed to assess the safety, tolerability, and activity of LB-100 administered for 3 consecutive days every 3 weeks. Pharmacokinetic studies were planned at the maximum tolerable dose (MTD). The starting dose, 0.25 mg/m², 1/15^{th} of the highest non-severely toxic dose in dogs, and plan of dose escalation was specified by the FDA. The study was approved by the human investigations committee at each study center and is registered at clinicaltrials.gov: NCT01837667.

LB-100 was supplied as a single use solution. Initially, LB-100 was administered in 50 mL of saline over 15 minutes. Because of a non-limiting reversible increase in serum creatinine at the 2.33 mg/m² level, LB-100 was subsequently administered in 500 mL normal saline over 2 hours. Dose escalation was prohibited within any cohort. Patients were eligible to receive up to 6 cycles of study therapy, unless unacceptable toxicity, disease progression, or inter-current illness required discontinuation. More than 6 cycles were allowed in the absence of progression and toxicity. Because of cardiac and renal toxicity at high doses in animal toxicology studies, patients had extensive monitoring including ECG, MUGA or echocardiogram, cardiac troponins, and BNP prior to every cycle. Blood chemistries, urinalysis, hematologic profile and vital signs were monitored prior to and on day 1, 3, 8, 15, and 22 of each cycle. Laboratory parameters were tabulated by maximum NCI-CTCAE (Version 4.0) severity grade. A safety review committee assessed all clinical data every 2 weeks and approved dose escalation between cohorts.

### Evaluation of Toxicity and Clinical Activity

Doses of LB-100 were escalated in groups of three patients. The first patient at a new dose level was observed for three weeks before treating the next two at that dose. When a potential DLT occurred, three new patients were entered at that dose. If another DLT occurred, three additional patients were treated at the previous non-DLT dose to determine the safety of that level for phase 2 trials.

Response to treatment was assessed using RECIST version 1.1. All patients with measurable disease, who completed 2 cycles of LB-100 and had at least 1 post-baseline tumor assessment, were evaluable for efficacy. Patients receiving any LB-100 were evaluable for safety. The severity of adverse events and laboratory abnormalities is reported according to NCI-CTCAE version 4.0 and coded using Medical Dictionary for Regulatory Activities.

### Outcomes

The main objective was to determine the safety, tolerability, and maximum tolerated dose of LB-100 given intravenously daily for three consecutive days every 3 weeks. The secondary objectives were to document any evidence of potential anti-tumor activity and obtain pharmacokinetic data on LB-100 and a metabolite, endothall, in patients receiving LB-100 at the MTD (Quang et al., 2016).

### Results

There were 29 patient entries over 7 dose escalations. One patient stopped treatment after one dose because of an acute infection and was reenrolled after recovery. Both courses were analyzed as separate patient entries. Two patients had dose limiting toxicity (reversible increases in serum creatinine or calculated serum creatinine clearance) at the 3.1 mg/m² level. Probable or possible study drug related Grade 3 adverse events occurred in 6 (20.7%) patients [anemia (n=2), decreased creatinine clearance, dyspnea, hyponatremia, lymphopenia]. Ten (50%) of 20 response-evaluable patients had stable disease for 4 or more cycles. One patient with pancreatic carcinoma had a partial response noted after 10 cycles that was maintained for 5 additional cycles. The other patients achieving stable disease had one of the following: fibrosarcoma, chondrosarcoma, thymoma, atypical carcinoid of lung, or ovarian, testicular, breast (n=2), and prostate cancer. The recommended phase 2 dose of LB-100 is 2.33 mg/m² daily for 3 days every 3 weeks.

### Patient Characteristics

Twenty-eight patients with advanced solid tumors were enrolled at four clinical sites. Their demographic features are listed in Table 1. Four patients were not evaluable for toxicity. Three of these patients had disease-associated complications prior to completing cycle 1. A fourth patient with atypical carcinoid of the lung was removed from study after one dose of LB-100 because of an acute infection; he was re-entered on study 7 weeks later and achieved stable disease for 5 cycles. Both courses were included in the analyses. None of these adverse events was considered related to drug administration.

TABLE 1
Patient Baseline Clinical and Demographic Characteristics
Study population ( n = 28)

| Sex | |
|---|---|
| Male | 14 (50.0%) |
| Female | 14 (50.0%) |
| Ethnic Origin | |
| White | 23 (82.1%) |
| Asian | 3 (10.7%) |
| Not Reported | 1 (3.6%) |
| Other | 1 (3.6%) |
| Age (years) | |
| N | 28 |
| Mean | 62.3 |
| Standard Deviation | 10.66 |
| Median | 64.0 |
| Minimum | 35 |
| Maximum | 79 |
| 8 to 64 | 15 (55.6%) |
| 65+ | 13 (46.4%) |
| Primary Site | |
| LUNG & BRONCHUS | 5 (17.9%) |
| LARGE INTESTINE, (EXCL. APPENDIX) | 5 (17.9%) |
| BREAST | 2 (7.1%) |
| CONNECTIVE & SOFT TISSUE | 2 (7.1%) |
| OVARY | 2 (7.1%) |
| TESTIS | 2 (7.1%) |
| APPENDIX | 1 (3.6%) |
| BONES & JOINTS | 1 (3.6%) |
| CORPUS UTERI | 1 (3.6%) |
| PANCREAS | 1 (3.6%) |
| PROSTATE GLAND | 1 (3.6%) |
| RECTUM | 1 (3.6%) |
| SMALL INTESTINE | 1 (3.6%) |
| THYMUS | 1 (3.6%) |
| UTERUS, NOS | 1 (3.6%) |
| VULVA, NOS | 1 (3.6%) |

### Dose Escalation and Toxicity

Twenty-four patients completed at least one 3-day cycle of LB-100. The tested dose levels were 0.25, 0.50, 0.83, 1.25, 1.75, 2.33, and 3.1 mg/m². There was no DLT during the first 6 dose levels. At the 3.1 mg/m² dose level, a patient with prostate cancer and one with chondrosarcoma had no DLT during 4 and 9 cycles of treatment, respectively. A third patient with ovarian cancer had a grade 3 increase in calculated creatinine clearance after cycle 1 with a return to normal by day 8 and received 3 more cycles at a reduced dose of 2.33 mg/m² before tumor progression. A fourth patient with fibrosarcoma had a grade 3 increase in calculated creatinine clearance after the first course. The creatinine returned to pretreatment value by day 21 and a second course at 2.33 mg/m² resulted in a grade 2 increase in creatinine clearance without other toxicity. The dose was decreased to 1.75 mg/m² and ten more cycles were administered without toxicity until progression after 36 weeks. Because 2/4 patients at 3.1 mg/m² had grade 3 increases in creatinine clearance during cycle one, three additional patients were evaluated at the preceding dose level of 2.33 mg/m². They had no limiting toxicity thereby establishing the MTD at that level. There was no symptomatic toxicity other than reversible mild to moderate fatigue. Adverse events possibly related to drug administration are listed in Table 2.

TABLE 2
Adverse Events in the Safety Population

| MedDRA Preferred Term [1][2] | Grade 1-2 | Grade 3 | Grade 4 | Grade 5 |
|---|---|---|---|---|
| Total Patients with Related Treatment- | 22 (75.9%) | 6 (20.7%) | 0 | 0 |
| Emergent Adverse Events [3] | | | | |
| FATIGUE | 8 (27.6%) | 0 | 0 | 0 |
| BLOOD CREATININE INCREASED | 5 (17.2%) | 0 | 0 | 0 |
| ASPARTATE AMINOTRANSFERASE INCREASED | 4 (13.8%) | 0 | 0 | 0 |
| HEADACHE | 3 (10.3%) | 0 | 0 | 0 |
| HYPERNATRAEMIA | 3 (10.3%) | 0 | 0 | 0 |
| HYPOALBUMINAEMLA | 3 (10.3%) | 0 | 0 | 0 |
| NAUSEA | 3 (10.3%) | 0 | 0 | 0 |
| PROTEINURIA | 3 (10.3%) | 0 | 0 | 0 |
| PYREXIA | 3 (10.3%) | 0 | 0 | 0 |
| ALANINE AMINOTRANSFERASE INCREASED | 2 (6.9%) | 0 | 0 | 0 |
| CONSTIPATION | 2 (6.9%) | 0 | 0 | 0 |
| NEUROPATHY PERIPHERAL | 2 (6.9%) | 0 | 0 | 0 |
| OEDEMA PERIPHERAL | 2 (6.9%) | 0 | 0 | 0 |
| SINUS TACHYCARDIA | 2 (6.9%) | 0 | 0 | 0 |
| ABDOMINAL DISCOMFORT | 1 (3.4%) | 0 | 0 | 0 |
| ABDOMINAL DISTENSION | 1 (3.4%) | 0 | 0 | 0 |
| ACCELERATED HYPERTENSION | 1 (3.4%) | 0 | 0 | 0 |
| ANAEMIA | 1 (3.4%) | 2 (6.9%) | 0 | 0 |
| ARTHRALGIA | 1 (3.4%) | 0 | 0 | 0 |
| BLOOD ALKALINE PHOSPHATASE INCREASED | 1 (3.4%) | 0 | 0 | 0 |
| BLOOD UREA INCREASED | 1 (3.4%) | 0 | 0 | 0 |
| CANDIDIASIS | 1 (3.4%) | 0 | 0 | 0 |
| CHEST PAIN | 1 (3.4%) | 0 | 0 | 0 |
| CHILLS | 1 (3.4%) | 0 | 0 | 0 |
| DECREASED APPETITE | 1 (3.4%) | 0 | 0 | 0 |
| DERMATITIS ACNEIFORM | 1 (3.4%) | 0 | 0 | 0 |
| DIARRHOEA | 1 (3.4%) | 0 | 0 | 0 |
| DIZZINESS | 1 (3.4%) | 0 | 0 | 0 |
| EJECTION FRACTION DECREASED | 1 (3.4%) | 0 | 0 | 0 |
| ELECTROCARDIOGRAM QT PROLONGED | 1 (3.4%) | 0 | 0 | 0 |
| GAIT DISTURBANCE | 1 (3.4%) | 0 | 0 | 0 |
| GASTROINTESTINAL DISORDER | 1 (3.4%) | 0 | 0 | 0 |
| GENERALISED OEDEMA | 1 (3.4%) | 0 | 0 | 0 |
| GINGIVAL PAIN | 1 (3.4%) | 0 | 0 | 0 |
| HYPERCALCAEMIA | 1 (3.4%) | 0 | 0 | 0 |
| HYPERKALAEMIA | 1 (3.4%) | 0 | 0 | 0 |
| HYPERTENSION | 1 (3.4%) | 0 | 0 | 0 |
| HYPOAESTHESIA | 1 (3.4%) | 0 | 0 | 0 |
| HYPOKINESIA | 1 (3.4%) | 0 | 0 | 0 |
| HYPOTENSION | 1 (3.4%) | 0 | 0 | 0 |
| HYPOXIA | 1 (3.4%) | 0 | 0 | 0 |
| INSOMNIA | 1 (3.4%) | 0 | 0 | 0 |
| MUCOSAL INFLAMMATION | 1 (3.4%) | 0 | 0 | 0 |
| MUSCLE TWITCHING | 1 (3.4%) | 0 | 0 | 0 |
| MUSCULAR WEAKNESS | 1 (3.4%) | 0 | 0 | 0 |
| NEUTROPENIA | 1 (3.4%) | 0 | 0 | 0 |
| OEDEMA | 1 (3.4%) | 0 | 0 | 0 |
| PAIN OF SKIN | 1 (3.4%) | 0 | 0 | 0 |
| PERIPHERAL COLDNESS | 1 (3.4%) | 0 | 0 | 0 |
| PERIPHERAL SENSORY NEUROPATHY | 1 (3.4%) | 0 | 0 | 0 |
| PLATELET COUNT DECREASED | 1 (3.4%) | 0 | 0 | 0 |
| PLEURAL EFFUSION | 1 (3.4%) | 0 | 0 | 0 |
| TACHYPNOEA | 1 (3.4%) | 0 | 0 | 0 |
| TREMOR | 1 (3.4%) | 0 | 0 | 0 |
| VOMITING | 1 (3.4%) | 0 | 0 | 0 |
| WEIGHT DECREASED | 1 (3.4%) | 0 | 0 | 0 |
| CREATININE RENAL CLEARANCE | 0 | 1 (3.4%) | 0 | 0 |
| DYSPNOEA | 0 | 1 (3.4%) | 0 | 0 |
| HYPONATRAEMIA | 0 | 1 (3.4%) | 0 | 0 |
| LYMPHOCYTE COUNT DECREASED | 0 | 1 (3.4%) | 0 | 0 |

[1] Number of Patients used as denominator to calculate percentages.
[2] Patients with multiple TEAEs are counted once within a Preferred Term.
[3] Treatment-Emergent Adverse Events (TEAEs) are defined as all AEs that occurred after the first dose of study medication or within 30 days post-treatment period.

### Pharmacokinetics

The plasma concentrations of LB-100 and endothall were measured (Quang et al., 2016) prior to and over 4 hours after completion of the 2-hour infusion at the MTD of 2.33 mg/m² of LB-100 on day 1 in one patient and on day 1 and 3 in two patients. The pharmacokinetics of LB-100 were similar on day 1 and 3 and were characterized by a low clearance, low volume of distribution, and a short half-life. Plasma concentrations of endothall were low throughout the infusion, being below the lower limit of detection (5 ng/mL) in one patient. In the other two patients, the maximal concentration of endothall (34.7 ng/mL) was observed at the last sampling time point (4 h), which precluded determination of its elimination half-life (Table 3).

TABLE 3
Pharmacokinetic parameters for LB-100 and endothall
LB-100

| | | | | | Apparent | Apparent | |
|---|---|---|---|---|---|---|---|
| Subject | Day | | | Dose | CL^{a} | Vₛₛ^{b} | T_{1/2} |
| Group | Nominal | Subject | Gender | (mg/m²) | (ng * h/mL) | (ng * h/mL) | (h) |
| 1 | 1 | 001-0030 | Male | 2.33 | 2.5 | 0.52 | 1.10 |
| | 3 | | | 2.33 | 5.7 | 1.10 | 0.95 |
| 1 | 1 | 002-0028 | Female | 2.33 | 2.7 | 0.65 | 1.35 |
| | 3 | | | 2.33 | 2.0 | 0.47 | 1.56 |
| 1 | 1 | 003-0029 | Female | 2.33 | 4.7 | 1.06 | 1.58 |

Endothall

| Subject | Day | | | Dose | Cmax | Tmax | Tin | AUC |
|---|---|---|---|---|---|---|---|---|
| Group | Nominal | Subject | Gender | (mg/m²) | (ng/mL) | (h) | (h) | (ng * h/mL) |
| 1 | 1 | 001-0030 | Male | 2.33 | N-D^{a} | N-D^{a} | ND^{b} | N-D^{c} |
| | 3 | | | 2.33 | N-D^{a} | N-D^{a} | ND^{b} | N-D^{c} |
| 1 | 1 | 002-0028 | Female | 2.33 | 11.5 | 4 | ND^{d} | 22 |
| | 3 | | | 2.33 | 34.3 | 4 | ND^{b} | 143 |
| 1 | 1 | 003-0029 | Female | 2.33 | 14.8 | 4 | ND^{b} | 28 |

^{a}Clearance value and
^{b}volume of distribution at steady-state represents a close approximation because the0 plasmaconcentration-time profile was only characterized through 4 hours after completion of the infusion.
^{a}Plasma concentrations of endothall were below the lower limit of quantification (5 ng/ml)
^{b}Terminal elimination half-life and
^{c}AUC values could not be defined.

### Evaluation of Clinical Activity

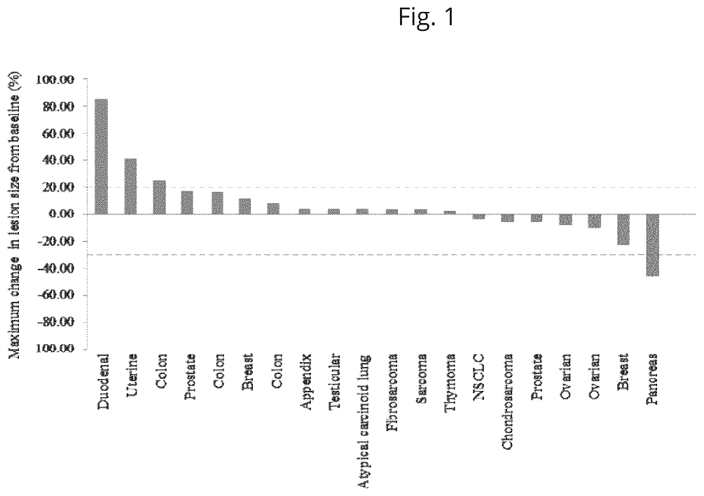

Of 20 patients with measurable disease, one patient with pancreatic cancer had a partial response, noted after 10 cycles and lasting for 5 more cycles, and 16 patients had no progression of their indicator lesion(s). They were removed from study for either the appearance of a new lesion or symptoms judged to represent clinical progression. Only 3 patients, one with duodenal and two with colonic adenocarcinomas, had significant increases in the size of their indicator lesion(s) by RECIST criteria (FIG. 1).

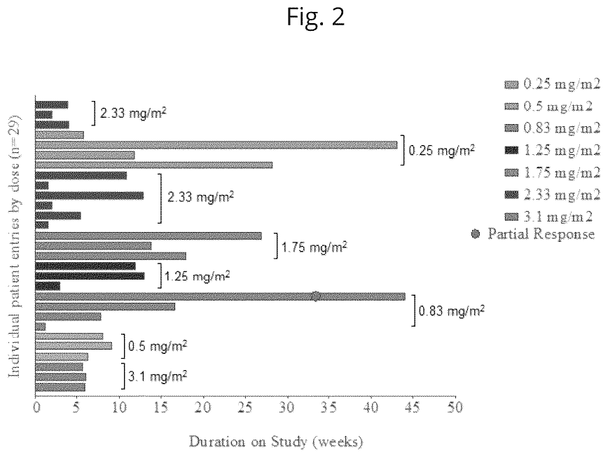

Achieving partial response or stability of disease was not clearly dose-dependent, occurring at 0.83 mg/m² in pancreatic cancer (15 cycles) and atypical carcinoid of the lung (5 cycles); at 1.25 mg/m² in breast cancer (4 cycles) and testicular cancer (5 cycles); and at 1.75 mg/m² in malignant thymoma (8 cycles) and ovarian cancer (6 cycles). At 3.1 mg/m², a patient with chondrosarcoma was stable for 8 cycles of LB-100 without any alteration in normal renal function whereas a patient with fibrosarcoma started at 3.1 mg/m² was stable for 12 cycles after two dose reductions (FIG. 2).

Conclusions: The safety, tolerability, preliminary evidence of anti-tumor activity, and novel mechanism of action of LB-100, support its continued development alone and in combination with other therapies.

### Discussion (Example 6)

The MTD of LB-100, a potent inhibitor of PP2A, was determined in patients with solid tumors. The recommended phase 2 starting dose is 2.33 mg/m² daily for 3 days every 3 weeks with escalation to 3.1 mg/m² in the absence of renal toxicity and de-escalation to 1.75 mg/m² or lower for renal toxicity in the event of stable or regressing disease. As patients had stability of disease and one patient with pancreatic cancer objective regression of pancreatic cancer at doses as low as 0.83 mg/m² daily for 3 days every 3 weeks, it is possible that optimum anti-cancer activity in humans may be considerable less than the MTD.

Ten (50%) of 20 patients receiving at least 2 cycles of LB-100 had stable disease for up to 15 cycles of therapy without limiting or cumulative toxicity. The mechanism underlying this phenomenon is not clear. PP2A activity is impaired or enhanced in many types of cancer by mutation or by increased expression of one or more of several endogenous PP2A inhibitors (Chang et al., 2015; Perotti et al., 2013; Seshacharyulu et al., 2013; Sangodkar et al; 2016). Since the single patient with pancreatic cancer in the present study had an objective response and otherwise stable disease for over 11 months, it is of special interest that marked overexpression of a regulatory subunit of PP2A associated with PP2A hyperactivity in a majority of human pancreatic cancers has recently been reported (Hein et al., 2016). Knockdown of this subunit, PR55α, in a human pancreatic cell line orthotopically implanted in nude mice, significantly reduced its tumorigenicity and metastatic potency (Hein et al., 2016).

On the other hand, without wishing to be bound to a specific theory, acquired deficits in PP2A activity may render tumors selectively vulnerable to further pharmacologic inhibition of PP2A. For example, in del(5q) myelodysplastic syndrome (MDS), an allele for the catalytic subunit of PP2A is deleted (Sallman et al., 2014). Lenalidomide, the standard agent for the treatment of MDS, was reported to be selectively cytotoxic to these PP2A haplo-insufficient del(5q)MDS cells by virtue of its moderate PP2A inhibitory activity (Sallman et al., 2014). PP2A inhibition also results in synthetic lethality of cancer cells that overexpress Mad2 (mitotic arrest deficiency protein 2) occurring in concert with mutations in Rb and/or p53 pathways (Bian et al., 2014; Schvartman et al., 2011). In the present study, the pancreatic cancer of the patient having a partial response markedly overexpressed Mad2.

Another potential mechanism by which single agent LB-100 may inhibit cancer progression is by enhancing cytotoxic T-cell function. Without wishing to be bound to a specific theory, the phosphatase activity of PP2A is important to CTLA-4-mediated T cell activation (Teft et al., 2009) and essential for regulatory T cell function (Apostolidis et al., 2016). In addition, inhibition of Ppp2r2d, a regulatory subunit of PP2A, enhances T-cell proliferation and cytokine production by a mechanism other than those of known negative regulators of T-cell function (Zhou et al., 2014). In the present study, however, no patient experienced toxicities suggestive of autoimmune activity that occurs with the currently approved compounds that induce immune checkpoint blockade.

The availability of a clinically safe inhibitor of PP2A provides an opportunity to exploit a long appreciated but neglected therapeutic target for cancer therapy. The current trial suggests that LB-100 alone has anti-cancer activity. Pharmacologic inhibition of PP2A, however, is likely to be most effective for cancer therapy when combined with cytotoxic drugs, particularly for tumors with acquired abnormalities in PP2A function and/or in the DNA-damage-repair pathway (Zhuang et al., 2009; Hong et al., 2015) and/or with other types of immune checkpoint inhibitors.

### Example 7. Administration of LB-100 in a Combination

An amount of compound LB-100 in combination with an anti-cancer therapy is administered to a subject afflicted with cancer. The amount of the compound and anti-cancer therapy is effective to treat the subject.

An amount of compound LB-100 in combination with an anti-cancer therapy is administered to a subject afflicted with cancer. The amount of the compound is effective to enhance the anti-cancer activity relative to the anti-cancer therapy alone.

### Example 8. PP2A and PD-1

Pharmacologic inhibition of PP2A could enhance immune activation and cancer immunotherapy. Inhibition of PP2A should enhance the cancer immunotherapy by directly increasing proliferation of conventional CD4 and CD8 T-cells and by impairing the immunosuppressive function of Tregs. It was hypothesized that LB-100 could augment the effect of immune checkpoint blockade. This is the first study demonstrating in a preclinical model, that pharmacologic inhibition of PP2A could synergize with immunotherapy.

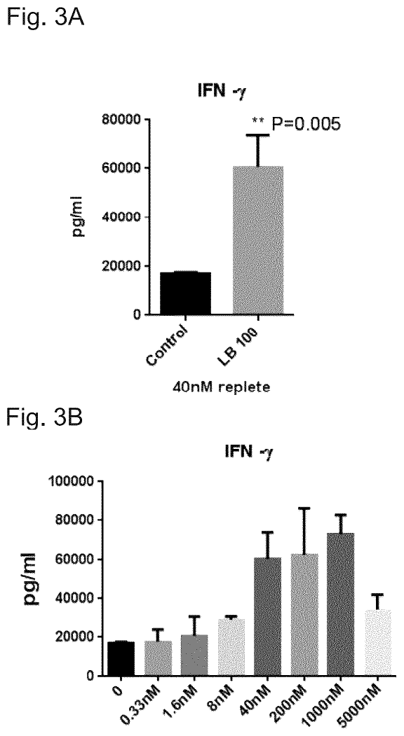

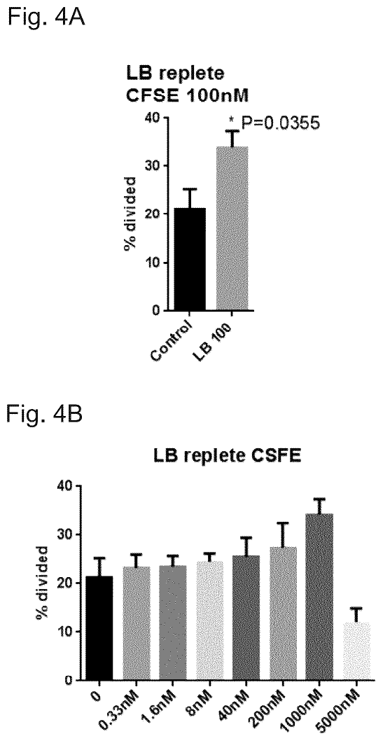

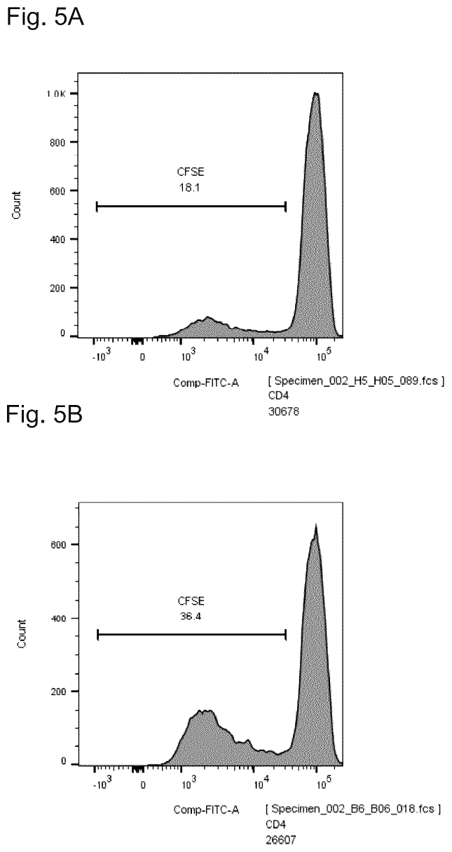

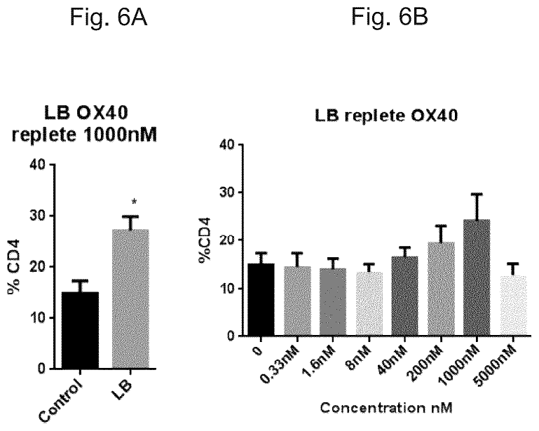

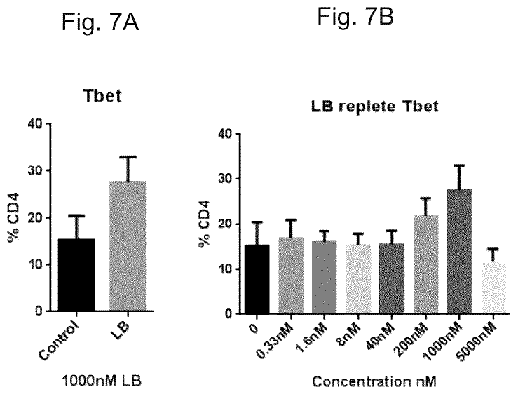

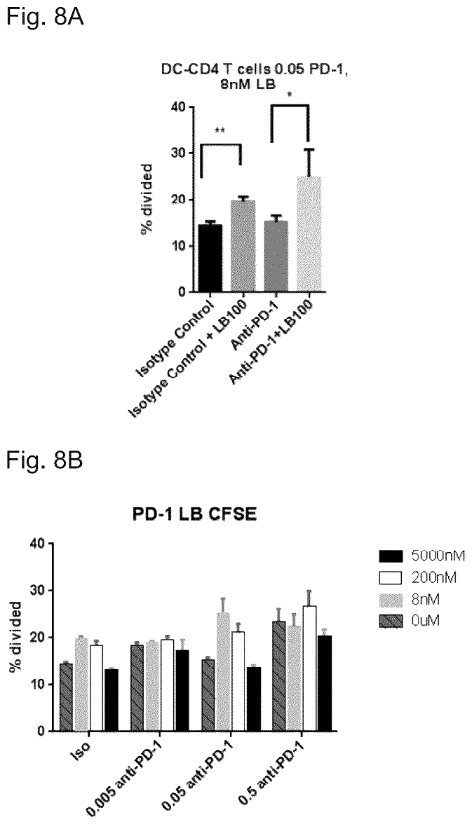

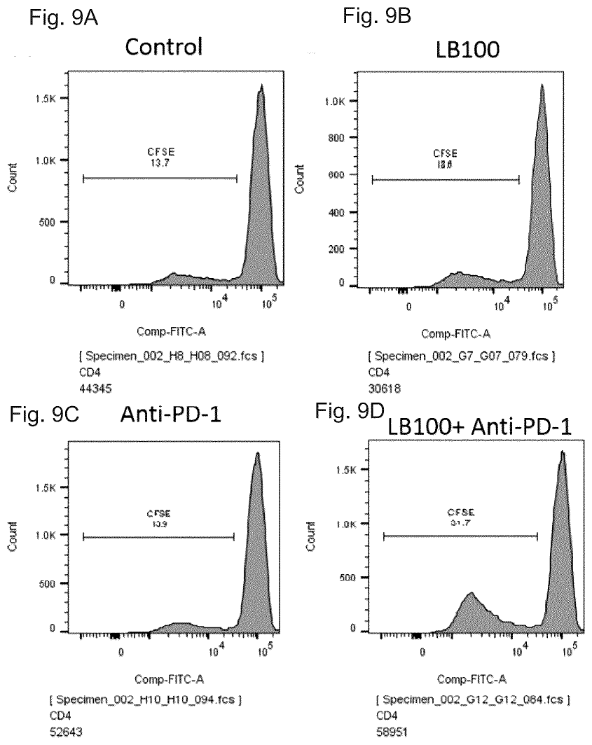

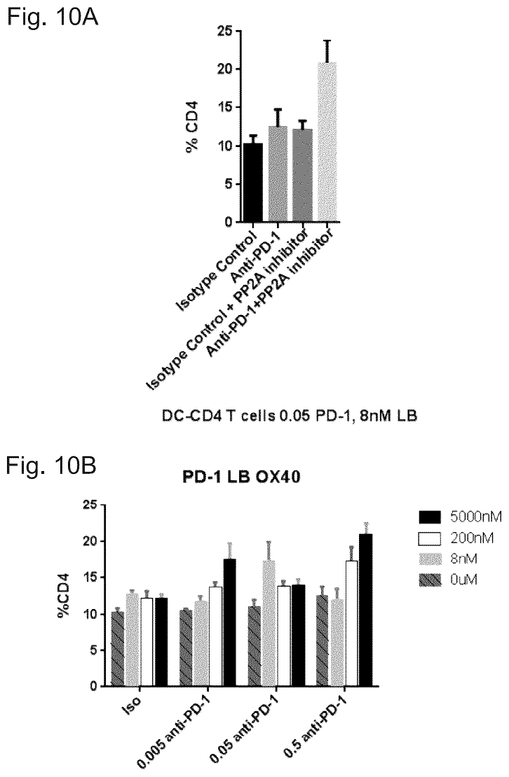

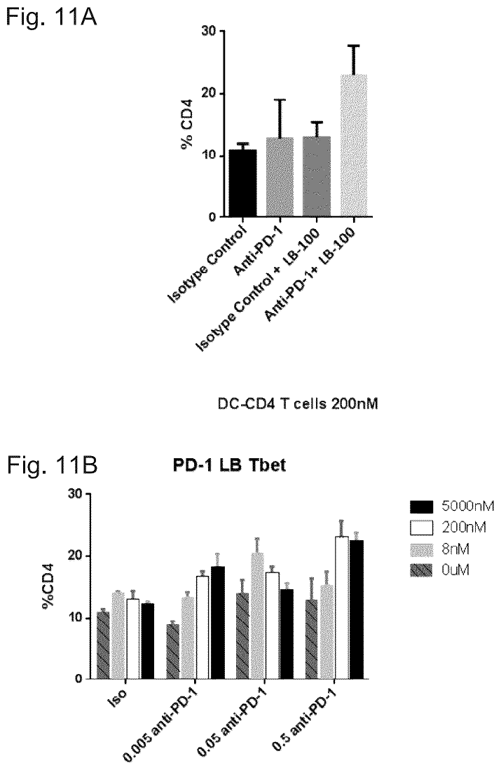

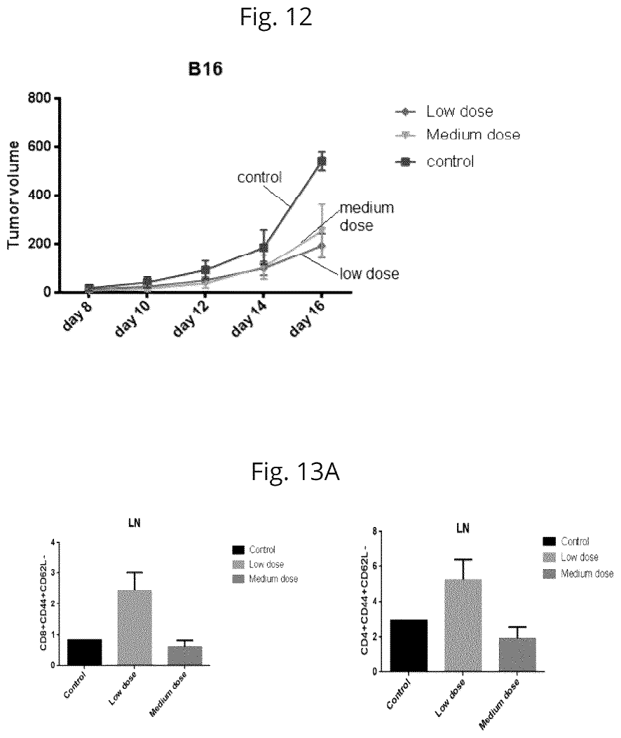

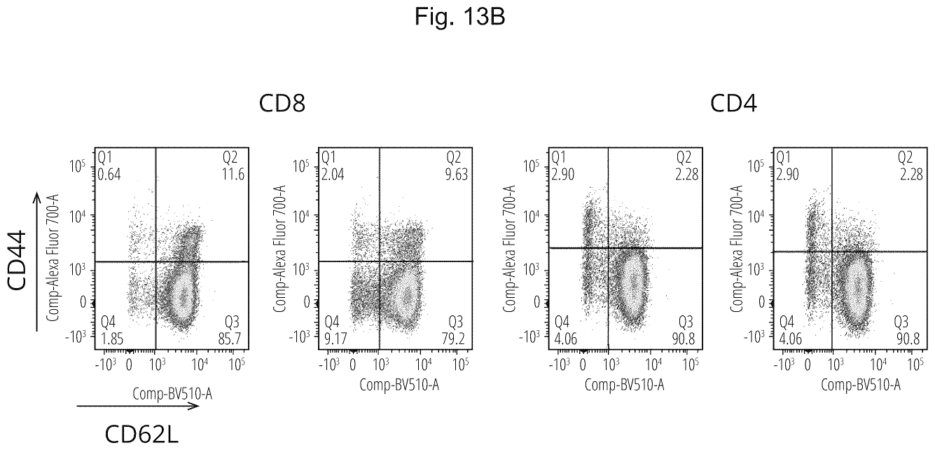

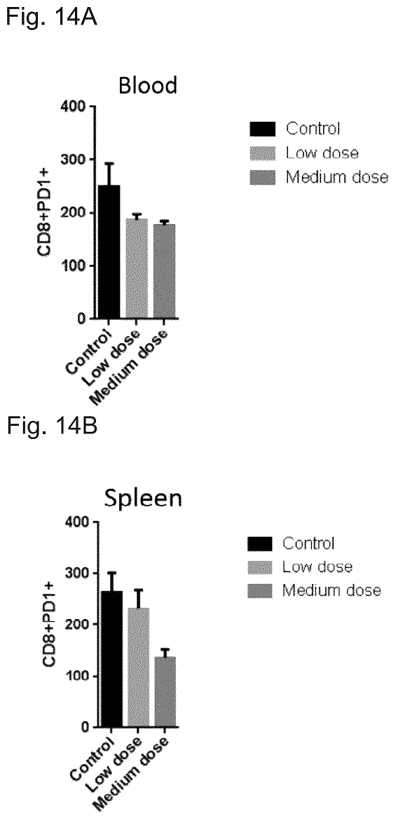

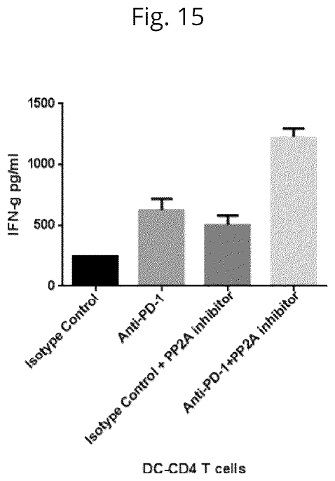

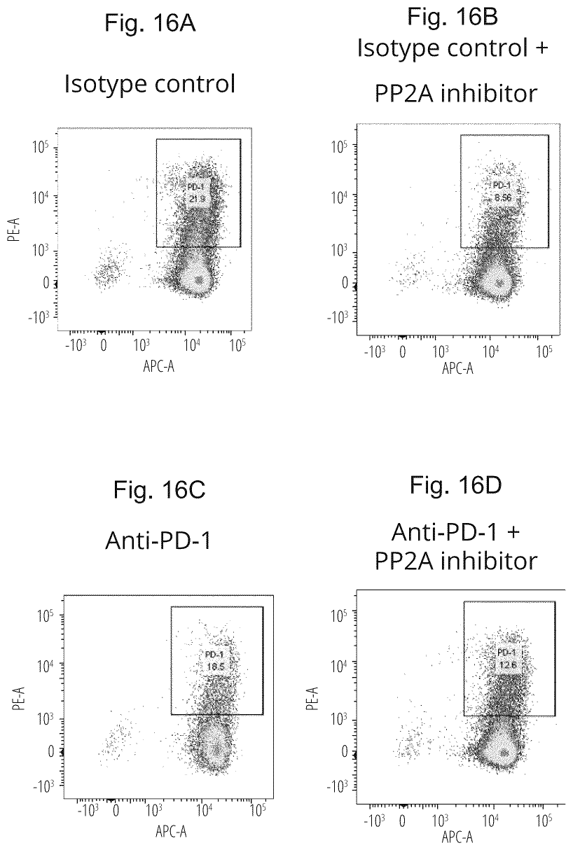

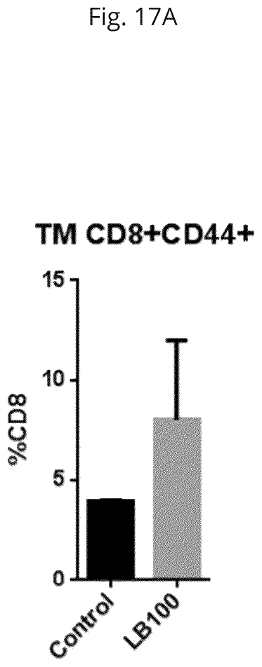

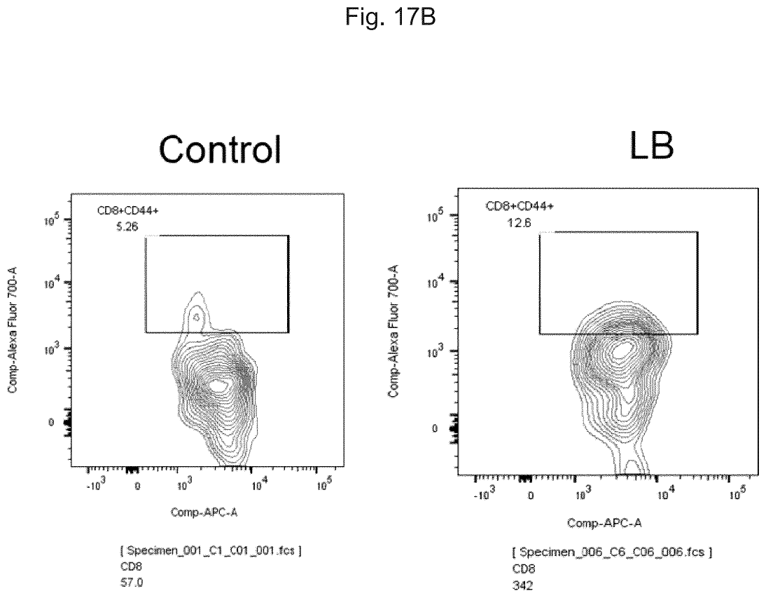

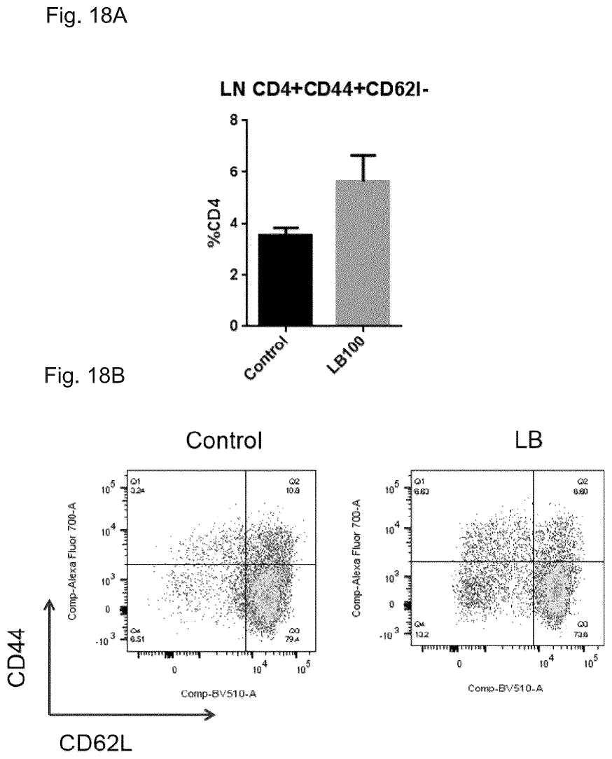

The effect of LB-100 on T-cells was assessed in human allogeneic mixed lymphocyte reactions, in which CD8+ or CD4+ T cells were co-cultured with autologous monocyte-derived dendritic cells. A dose dependent increase was found in T cell proliferation in CD8+ and CD4+ cells (FIGS. 4A-B and 5A-B) and an increase in IFNγ secretion in CD4+ T cells (FIGS. 3A-B). A dose dependent increase was found to enhance co-stimulatory molecule OX40 expression on T cells (FIGS. 6A-B) and Tbet, transcription factor to drive IFNγ production in CD4+ T cells (FIGS. 7A-B). The effect of LB-100 plus anti-PD-1 antibody was investigated on CD4+ T cells in the same assay. The combination enhanced proliferation (FIGS. 8A-B, 9A-D), OX40 expression (FIGS. 10A-B), Tbet expression (FIGS. 11A-B), and IFNγ production compared to anti-PD-1 alone (FIG. 15).

### Example 9. LB-100 Inhibition and PD-1 Blockades Elicit Durable CD8+ T Cell-Mediated Tumor Rejection

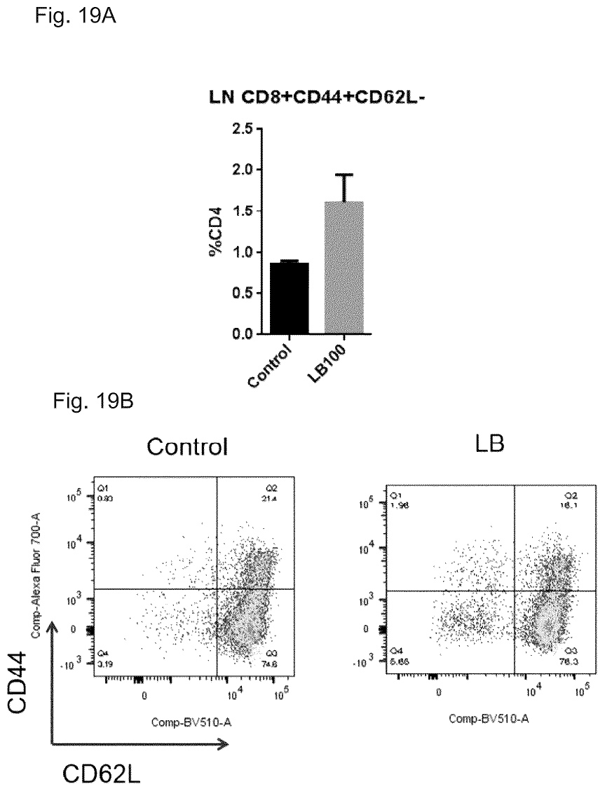

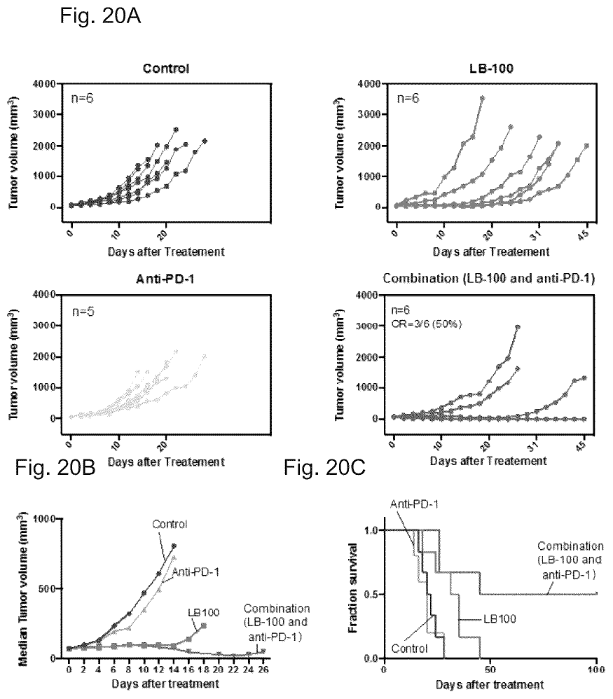

To test the hypothesis that inhibition of PP2A with L13-100 can enhance immune-mediated antitumor responses, mice were implanted with CT26 tumor cells. CT26 is a murine colon adenocarcinoma expressing low level of PD-L1 and is resistant to anti-PD1 therapy, After about 13 days, mice with tumor size between 30-100 mm³ were randomized into four treatment groups (PBS control, LB-100 only—0.16 mg/kg, anti-PD-1 only—10 mg/kg, or a combination of both). Treatment was given every 2 days for a total of 28 days. Tumor size was assessed every two days (FIGS. 20A-C). Single agent treatment with anti-PD1 was ineffective in reducing tumor burden or increasing survival. LB-100 alone was able to increase median survival from 21 to 33 days (p=0.02). The combined treatment significantly decreased mean tumor volume by 70% compared to control (p<0.01) on day 14 after treatment. Median survival also increased from 21 to 72 days (p<0.01). More strikingly, 50% of mice achieved complete regression (CR) of tumors with no evidence of disease. This response was durable after completion of treatment.

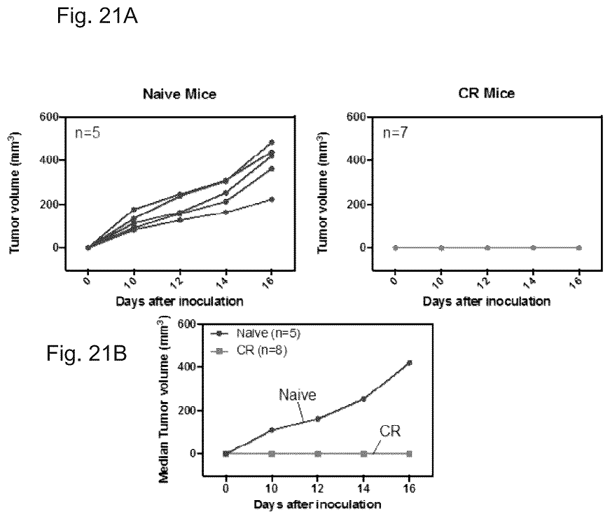

It was next examined whether mice that achieved CR from combination therapy developed long-term immune memory. About 60 days after initial inoculation, cured mice were re-inoculated with same CT26 cells (FIGS. 21A-B). None of the mice (n=8) developed tumor on re-challenge. CT26 naïve mice were inoculated at the same time to serve as control. This result indicates mice cured by the combination therapy were able to establish long-term memory to tumor specific antigen.

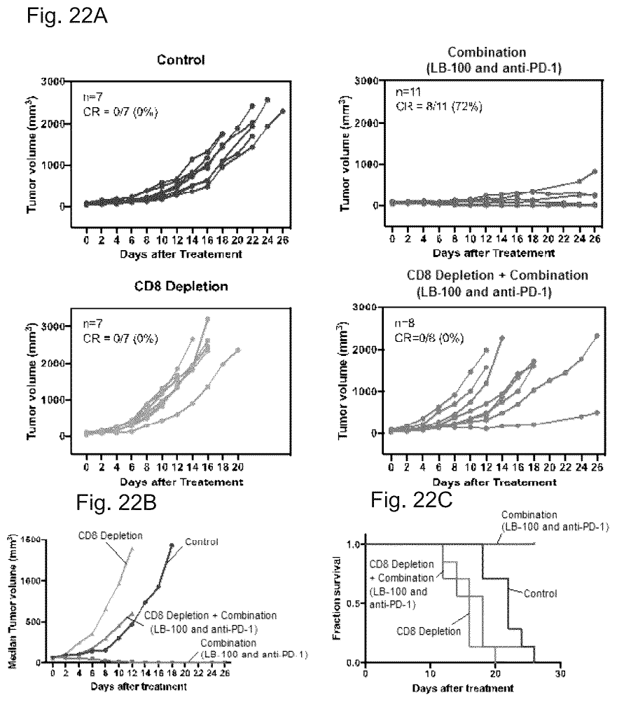

It was then explored whether the synergistic effect of LB-100 with anti-PD-1 that resulted in tumor regression is mediated by CD8 T-cells. Mice bearing CT26 tumors were subjected to CD8 ablation using depleting antibiotics prior to initiation of treatment. CD8+ T cells ablation was confirmed with FACS of splenocytes 3 days after treatment. With CD8 depleted, the combination therapy failed to elicit anti-tumor response. 0% of CD8+ depleted mice receiving combination achieve CR compared to 72% in the CD8+ non-depleted group (FIGS. 22A-C). Taken together, these results demonstrated that the effect of LB-100 is mediated by the immune system rather than a direct cytotoxic effect on the tumor.

### Methods

Reagents—LB-100 was provided by Lixte Biotechnology. Anti-mouse PD-1, Clone RMP1-14, antibody was purchased from BioXcell. The following monoclonal antibodies (mAbs) were used for flow cytometry: rat anti-mouse CD4-BV421, rat anti-mouse CD3-PE, rat anti-mouse CD8a-Alexa 647, rat anti-mouse CD45-BV785, rat anti-mouse IFNg-FITC, rat anti-mouse FOXP3-Alexa 647. These antibodies were purchased from Biolegend.

Cell Lines and Mice—CT26.CL25 colon carcinoma line was obtained from ATCC. Tumor cells were cultured in complete medium (RPMI 1640; Cellgro) containing 10% (vol/vol) FBS (Thermofisher), 100 U/mL penicillin, 100 μg/mL streptomycin (Gibco). Six- to 8-week-old female BALB/c were purchased from Charles River. Mice were housed in the Laboratory Animal Facility of the National Institutes of Health (Bethesda, MD). All experiments were approved by the National Institutes of Health Office of Animal Care and Use and conducted in accordance with National Institutes of Health guidelines.

Tumor Inoculation and Animal Studies—CT26 tumor cells (0.5×10⁶) were injected subcutaneously in the right side of the abdomen. LB-100 and anti-PD-1 was injected by the i.p. route at a dose of 0.16 mg/kg and 10 mg/kg respectively beginning on day 11 after tumor implantation and continued every 2 d for 28 d. Tumor size were monitored with a digital caliper every 2-3 d and expressed as volume (length×width²×0.5). Animals whose tumors became ulcerated/necrotic or a tumor burden exceeding 2,000 mm3 or with a largest diameter greater than 2 cm were euthanized.

Depletion of CD8 T cells. Anti-CD8 (clone 53.6.7) mAbs (BioXcell) were injected 2 d and 1 d before therapy, on the day therapy and at 5 and 8 d after beginning therapy. Dose was 0.1 mg per injection.

Tumor Rechallenge Studies. Naive BALB/c mice and mice previously cured with combination treatment as described above were inoculated with CT26 cells into the left (not previously inoculated) thoracic flank. Tumors were measured two times per week as described above. Animals whose tumors became ulcerated/necrotic or tumor burden exceeded 2,000 mm3 or had a largest diameter greater than 2 cm were euthanized.

### Example 10. LB-100 and PD-1 Blockades Regulates Tumor Infiltrating Lymphocytes (TIL)

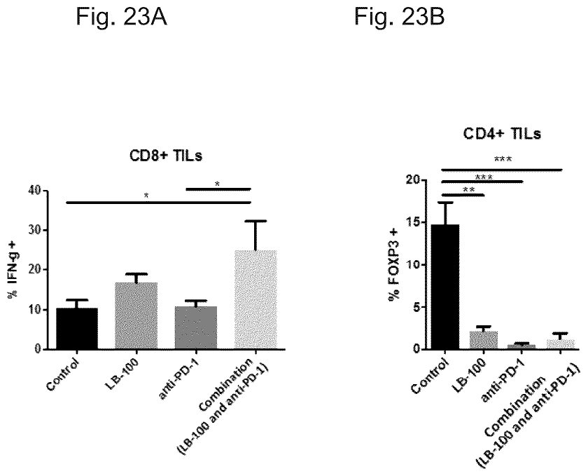

The treatment effect was assessed on tumor infiltrating lymphocytes (FIGS. 23A-B). CT26 tumor-bearing mice were treated with LB-100 and/or anti-PD-1 antibodies as above. After 12 days of treatment, tumors were analyzed by FACS. Tumor-infiltrating CD8+ T cells from mice treated with the LB-100 and anti-PD-1 antibody combination showed a significant increase in IFNg+CD8+ T cells compared to cells from control animals and animals treated with anti-PD-1 alone (25.3% compared to 11.0% and 10.5% respectively, p=0.05). Since IFNγ is the most critical cytokine mediating anti-tumor response, this result is a functional confirmation that the combination treated mice have enhanced immunity against the implanted tumors. In addition, given the known importance of PP2A in Treg, the effect of LB-100 and/or anti-PD-1 treatment was examined on the amount of Tregs present in the tumor. LB-100 alone significantly depleted Tregs in the tumor environment (2.1% compared to 14.7% in control). This effect is similar in degree to the effect on Tregs depletion by anti-PD-1 or the combination. The fact that LB-100 alone can deplete Tregs suggests a possible mechanism explaining the small but significant survival benefit observed in the LB-100 alone treatment group.

Furthermore, mice achieving CR were resistant to tumor growth when re-inoculated with CT26 cells. Mice subjected to CD8+ T cell ablation using depleting antibodies, were unable to reject CT26 tumors—0/8 (0%) despite treatment with combination therapy, indicating that the anti-tumor effect of LB-100 with anti-PD-1 treatment is CD8+ T cell mediated. In conclusion, in a syngeneic animal model, the PP2A inhibitor, LB-100, has synergistic potential in conjunction with checkpoint blockade supporting investigation of its ability to enhance immunotherapy in the clinic.

In summary, it has been demonstrated in this pre-clinical model that LB-100 when combined with anti-PD-1 has a robust and synergistic effect that results in complete regression of a significant portion of treated mice. This effect is mediated by adaptive immunity through CD8+ T cells. There is also establishment of immune memory associated with regression of tumor. This is the first report of using pharmacologic inhibition of PP2A as a target for enhancing immunotherapy.

Tumors often developed multiple mechanisms to evade the immune system, one of which is expression of PD-1 of T cells, which effectively inhibits T-cells from attacking the tumor. Anti-PD-1 abrogates this inhibitory signal, thereby allowing the T-cell to recognize and eradicate the tumor. LB-100, an inhibitor of Protein Phosphatase 2A (PP2A), was found to have dramatic antitumor effect in a preclinical model of colon cancer. This effect was found to be mediated by enhancement of the immune system.

### Example 11. Administration of LB-100 in Combination with a PD-1 and/or PD-L1 Checkpoint Inhibitor

An amount of compound LB-100 in combination with a PD-1 and/or PD-L1 checkpoint inhibitor is administered to a subject afflicted with cancer. The amount of the compound and inhibitor is effective to treat the subject.

An amount of compound LB-100 in combination with a PD-1 and/or PD-L1 checkpoint inhibitor is administered to a subject afflicted with cancer. The amount of the compound is effective to enhance the anti-cancer activity of the PD-1 and/or PD-L1 checkpoint inhibitor.

An amount of compound LB-100 in combination with a PD-1 and/or PD-L1 checkpoint inhibitor is administered to a subject afflicted with melanoma. The amount of the compound and inhibitor is effective to treat the subject.

An amount of compound LB-100 in combination with a PD-1 and/or PD-L1 checkpoint inhibitor is administered to a subject afflicted with melanoma. The amount of the compound is effective to enhance the anti-cancer activity of the PD-1 and/or PD-L1 checkpoint inhibitor.

### Example 12: Assessment of Efficacy of LB-100 as Add-on Therapy to Atezolizumab, Nivolumab or Pembrolizumab

The add-on therapy provides a synergistic effect, and allows for lower doses with reduced side effects.

Periodic administration of LB-100 as an add-on therapy for a human patient afflicted with melanoma who is already receiving Atezolizumab, Nivolumab or Pembrolizumab provides a clinically meaningful advantage and is more effective (provides at least an additive effect or more than an additive effect) in treating the patient than when Atezolizumab, Nivolumab or Pembrolizumab is administered alone (at the same dose).

Periodic administration Atezolizumab, Nivolumab or Pembrolizumab as an add-on therapy for a human patient afflicted with melanoma who is already receiving of LB-100 provides a clinically meaningful advantage and is more effective (provides at least an additive effect or more than an additive effect) in treating the patient than when LB-100 is administered alone (at the same dose).

The add-on therapies also provide efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in increasing the amount of cytotoxic T cells in patients with melanoma;
2. The add-on therapy is more effective (provides an additive effect or more than an additive effect) in slowing the progression of melanoma in patients with melanoma; and/or
3. The add-on therapy is more effective (provides an additive effect or more than an additive effect) in reducing the function of PD-1 and or PD-L1 in the melanoma patients.

### Example 13: Pharmacological Inhibition of Protein Phosphatase-2A with LB-100 Achieves Durable Immune-Mediated Anti-Tumor Activity When Combined With PD-1 Blockade

The present example demonstrates, inter alia, that a small molecule PP2A inhibitor, LB-100, when combined with anti-PD1 (aPD-1) blockade synergistically elicits a durable immune-mediated anti-tumor response in the CT26 colon cancer model. This effect was T cell dependent, leading to a striking regression of a significant proportion of tumors. Analysis of tumor lymphocytes demonstrated enhanced infiltration of effector T cells and depletion of suppressive regulatory T cells resulting in a marked increase in effector-to-regulatory T cell ratios. Clearance of tumor established antigen-specific secondary protective immunity. A synergistic effect of LB-100 and aPD-1 blockade was also observed in the B16 melanoma model. In addition, described herein is the finding that LB-100 specifically activated mTORC1 signaling pathway resulting in decreased differentiation of naïve CD4 cells into regulatory T cells. There was also found an increased expression of Th1 and decreased expression of Th2 cytokines. These data highlight the translational potential of PP2A inhibition in combination with checkpoint inhibition.

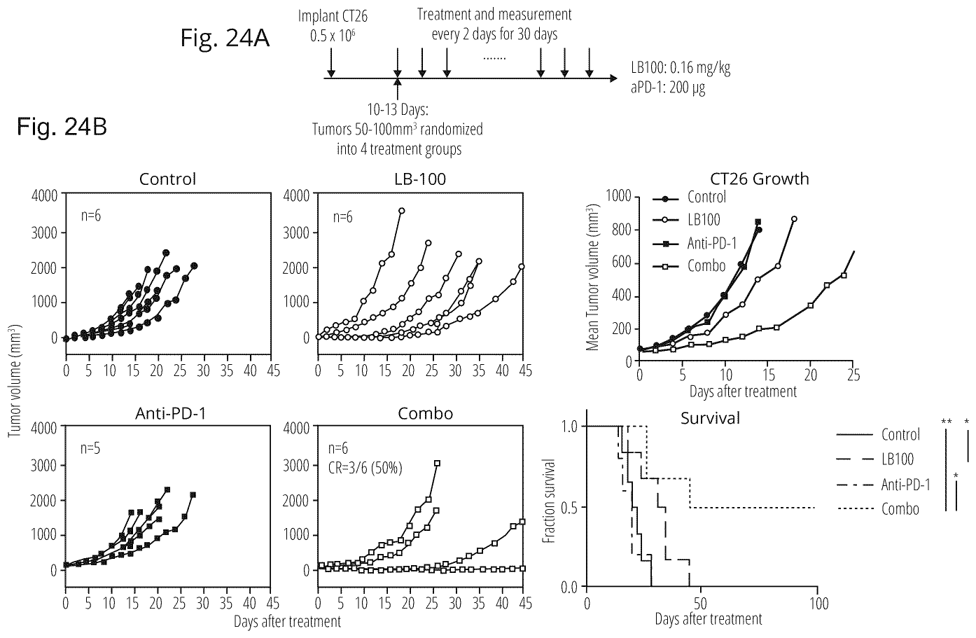

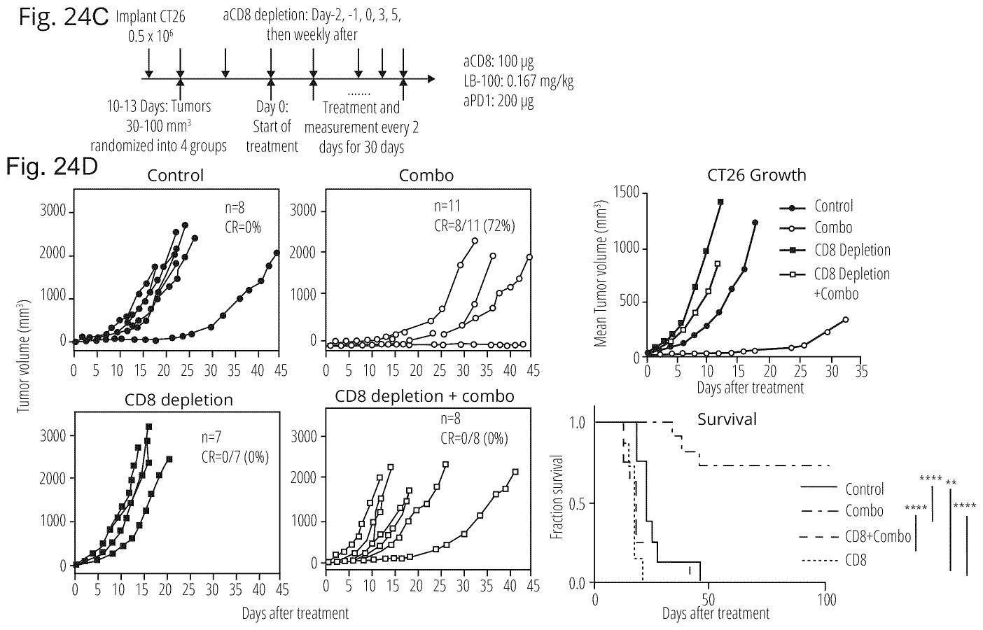

LB-100 and aPD-1 combination treatment synergistically elicit rejection of CT26 tumors: To test the hypothesis that PP2A inhibition synergizes with aPD-1 therapy in vivo in aPD-1 refractory tumors, a CT26 tumor was used, which is a murine colorectal carcinoma with high PD-L1 expression but limited response to aPD-1 therapy. Mice were inoculated with CT26 tumor cells (0.25×10⁶). After 10-13 days, mice with tumors reaching 50-100 mm³ in size were randomized into four treatment groups: control (PBS), aPD-1, LB-100 and the combination of aPD-1 and LB-100. Treatments were administered every 2 days for 30 days. Tumor growth was assessed every 2 days (FIG. 24A). In this model, LB-100 alone did not significantly decrease tumor growth, but did extend median survival (33 vs 21 days, p=0.02). Additionally, aPD1 alone had no effect on tumor growth or survival. The combination of LB-100 and aPD1, however, resulted in striking regression of a significant portion of tumors, with 50% achieving complete regression (CR) for the duration of the study. There was a significant difference in tumor size at day 8 after treatment (p<0.05) and significant increase in survival (p<0.005) between the combination and control treatment arms (FIG. 24B).

Effect of LB100 and aPD-1 combination is dependent on CD8 T-cells: Next examined was whether the synergic effect of the LB-100 and aPD-1 combination resulting in durable tumor regression was an immune mediated process. CT26-tumor bearing mice were subjected to CD8+ T cell ablation using depleting antibodies prior to and during treatment with LB-100 and aPD-1 (FIG. 24C). Peripheral CD8+ depletion was confirmed 5 days after treatment by FACS (data not shown). When depleted of CD8+ T-cells, the LB-100 and aPD-1 combination did not elicit tumor rejection (0 vs 72%, p=0.0015) (FIG. 24D). Mean tumor volume was increased 13-fold 10 days after treatment in the combination group with CD8 depletion compared to tumor volume in the non-depleted group (612 vs 46 mm³, p<0.001). Survival was also significantly decreased with CD8 depletion (p<0.0001). CD8 T cell depletion alone had a small deleterious effect compared to control in both tumor growth and survival, suggesting a baseline level of CD8+ T cell mediated immunity served to limit CT26 growth in baseline conditions. These data indicated that LB-100 with aPD-1 synergy is dependent upon CD8+ T cell-mediated adaptive immunity and not a direct effect of PP2A inhibition of tumor growth.

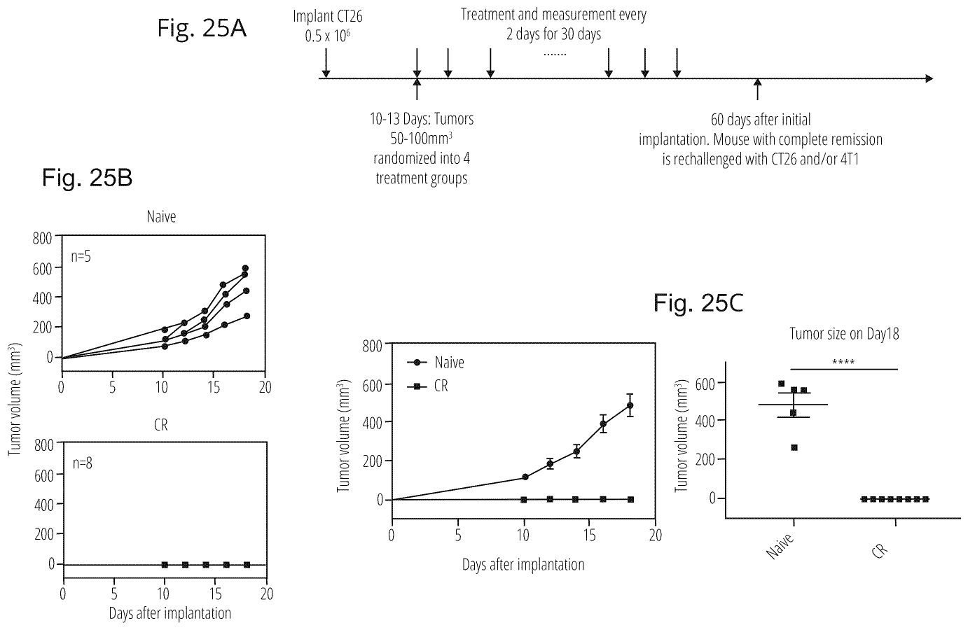

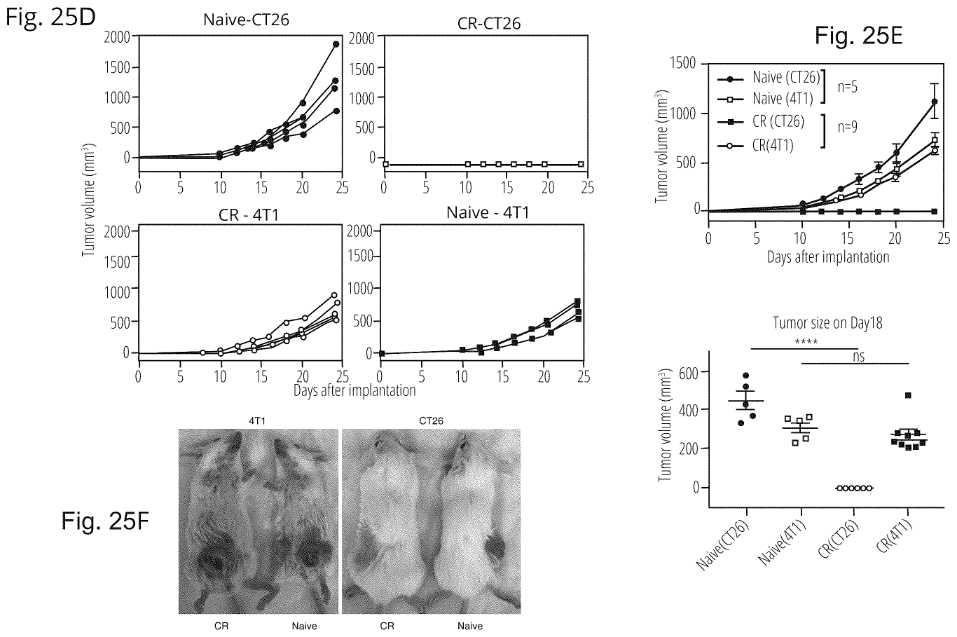

Mice cured by the combination therapy develop antigen-specific long-term memory: The hallmark of a successful adaptive immune response is the establishment of immunologic memory. The following experiment tested mice that experienced a complete response (CR) for their secondary protective anti-tumor response. Mice were re-challenged with CT26 cells about 60 days after initial tumor implantation (FIG. 25A). These mice were completely resistant to CT26 cell re-challenge (FIG. 25B). The average tumor size at day 18 after (re-)implant was 480 mm³ in naïve compared to 0 mm³ in CR mice (p<0.0001) (FIG. 25C).

Next, whether the protective secondary immunologic response was specific to CT26 tumors was tested. After about 60 days from initial implantation, CR mice were re-challenged with both CT26 cells in the flank and 4T1 cells, an unrelated murine breast cancer cell line, in the mammillary fat pad (FIG. 25D). Mice with CR were resistant to CT26 but not to 4T1 cells. Eighteen days after inoculation, there was no difference in 4T1 tumor volume between naïve and CR mice, while CT26 failed to grow in CR mice (FIG. 25E-F). This result indicates that the secondary memory response is specific to CT26-expressed antigens.

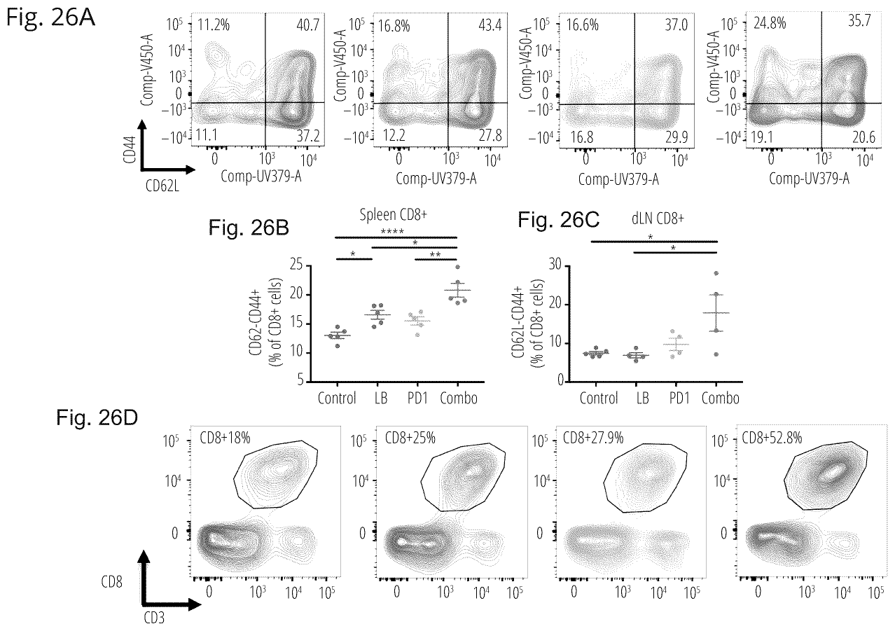

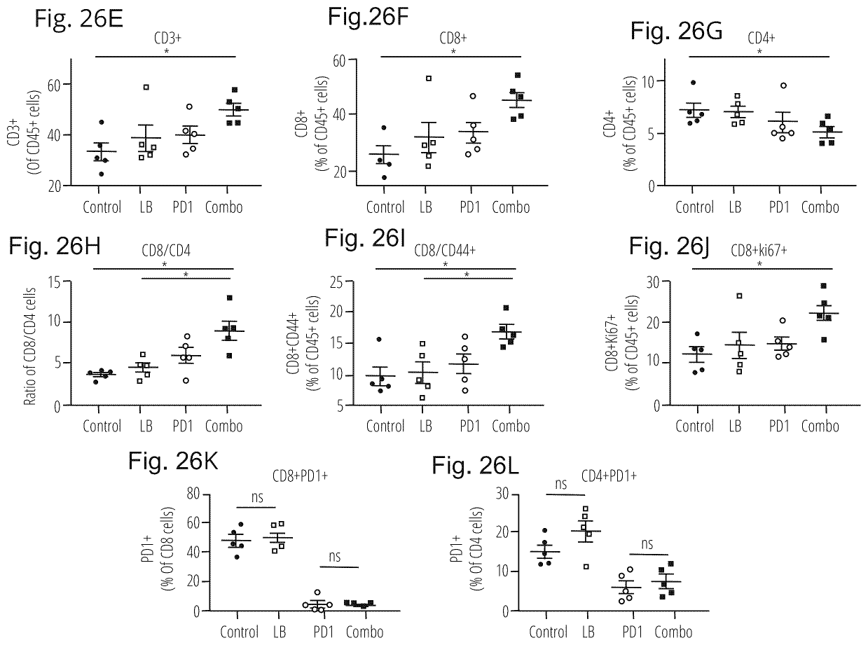

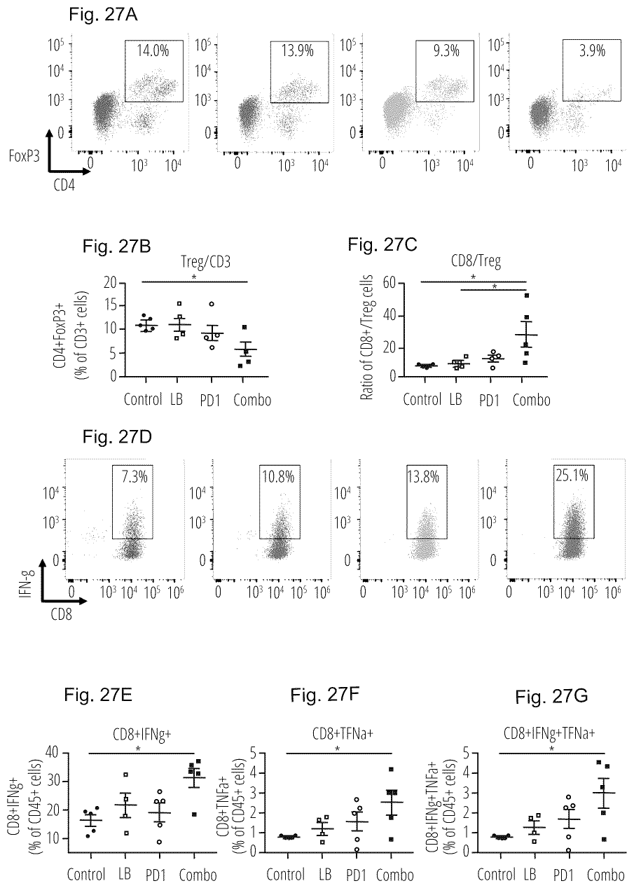

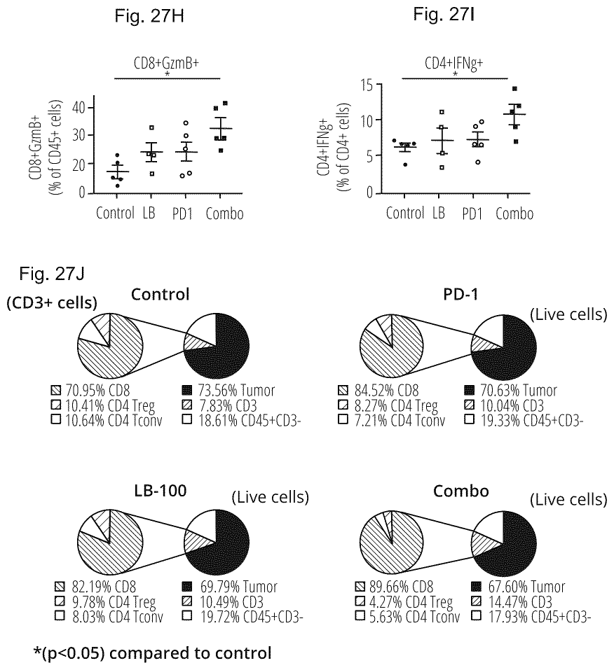

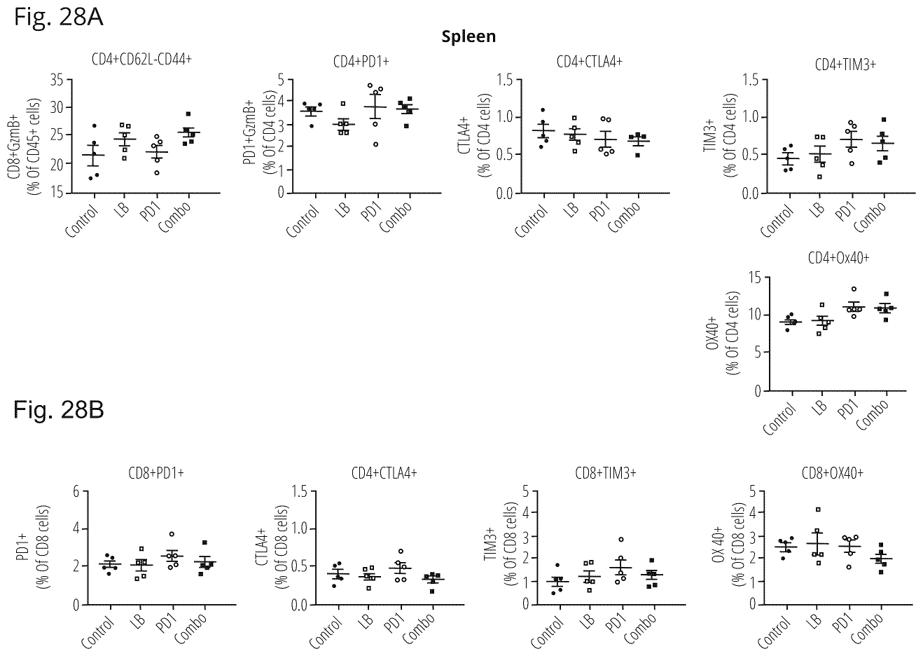

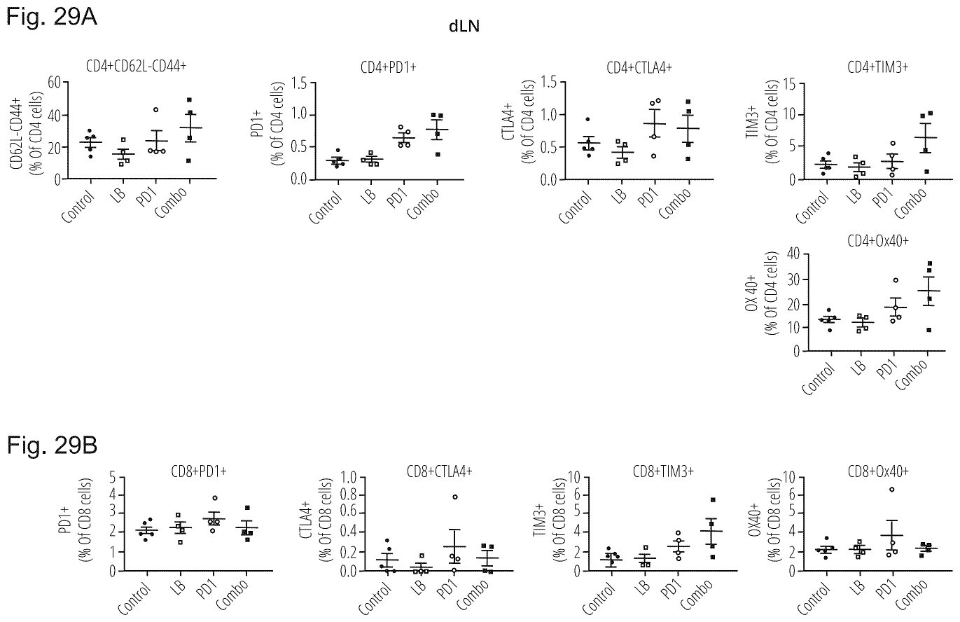

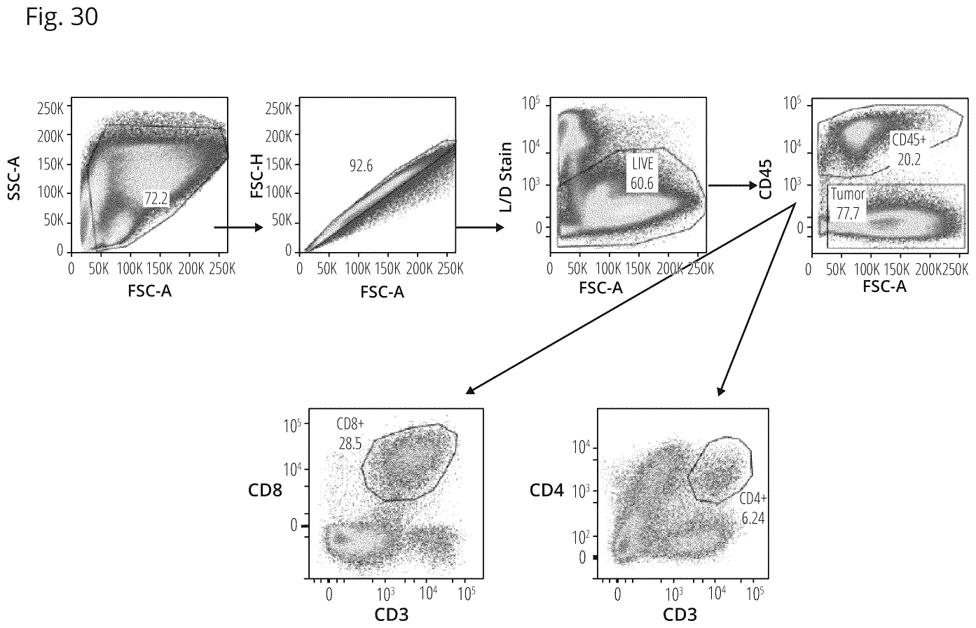

Enhanced activation of lymphocytes with combination treatment: To address the cellular mechanism mediating tumor rejection by the LB-100/aPD-1 combination, the status of the immune system in the secondary lymphoid organs and in the tumor were examined. Mice were implanted with CT26 tumors and treated with LB-100 and/or aPD-1 as described above. On day 3, after two treatments, the spleens, tumor draining lymph nodes (dLN) and tumors were harvested and analyzed by flow cytometry (FIG. 26-27). In the secondary lymphoid tissue, a greater activation of CD8+ T cells in mice treated with the combination regimen compared to controls was observed, as indicated by greater frequency of CD44+CD62L-CD8+ T cells (FIG. 26A-C). In the spleen, treatment with LB-100 alone resulted in a small increase in CD44+CD62L-CD8+ T cells (from 13.0 to 16.6%, p<0.05) but the combination treatment resulted in a greater increase than either LB-100 or aPD1 alone (20.8 compared to 16.6 and 15.5% respectively, p<0.05 and p<0.005) (FIG. 26B). Similarly, CD44+CD62L-CD8+ T cells were increased in the dLN of mice treated with the combination compared to control (from 7.4 to 17.9%, p<0.05) (FIG. 26C). There was no difference in frequency of CD44+CD62L− subset in CD4+ T cells in both the spleen and dLN (FIGS. 28A and 29A). Immune check point markers, including expression of PD-1, CTLA4, TIM3 and Ox40 on CD8+ and CD4+ T cells were examined in the tumor draining lymph node and spleen (FIGS. 28 and 29). There was no difference in the expression of these markers except for a small but significant increase in PD-1 expression in aPD-1 treated CD4+ T cell in the dLN; however, LB-100 alone or in combination did not further alter PD-1 expression (FIG. 29A).

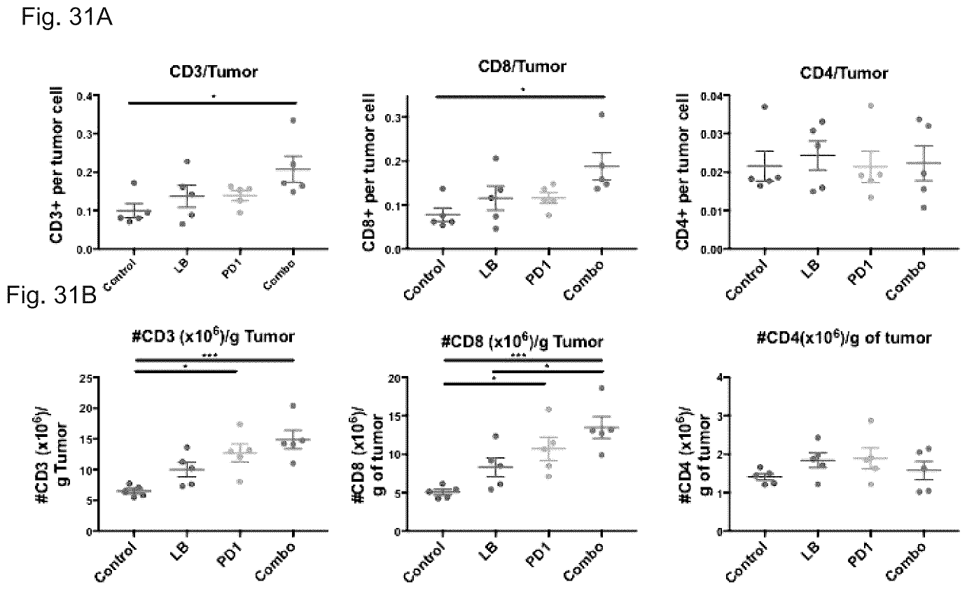

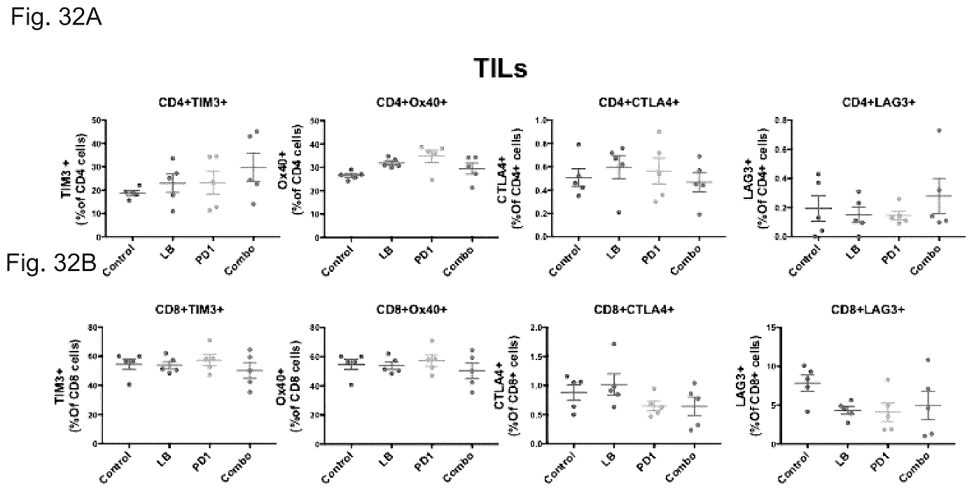

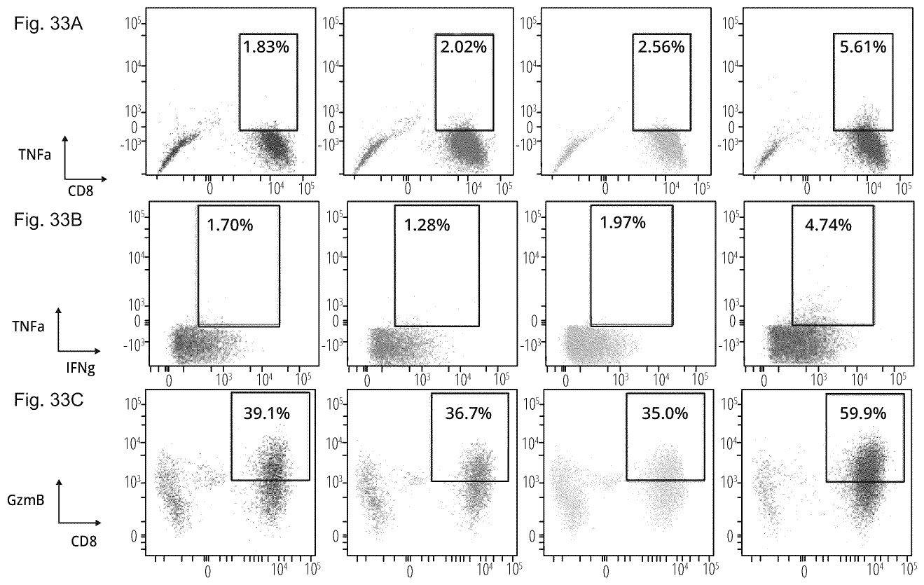

A comprehensive analysis of the tumor infiltrating lymphocytes (FIG. 26-27, 30) was next performed. First, the absolute percentage of CD45+ cells was examined. There was no significant difference among the treatment groups. However, within the CD45+ population, there was a significant increase in CD3+ T cells in the combination treatment compared to control (from 33.3 to 49.9%, p<0.05) (FIG. 26E). More importantly, this increase in CD3+ T cell population was attributed to a significant increase of CD8+ T cells (FIG. 26D), whether normalized to CD45+ cells (from 25.9 to 45.3%, p<0.01) (FIG. 26F) or number of tumor-resident cells (from 8 to 19%, p<0.05) (FIG. 31A). A similar trend was observed in CD8+ T cells normalized to tumor weight (FIG. 31B). Instead, the CD4+ T cell population remained unchanged (FIGS. 26G, 31), resulting in a marked increase in CD8/CD4 ratio (from 3.6 to 9.0, p<0.001) (FIG. 27H). This indicated that LB-100/aPD-1 combination resulted in enhanced CD8+ T cells trafficking to the tumor, which has consistently been shown to be one the most important predictors of response to immunotherapy. Further examined was the subpopulation of CD8+ TILs by labeling the effector phenotype marker CD44+. There was a significant increase in CD8+CD44+ T cells in mice treated with the combination relative to control (9.8 to 17.1%, p<0.01) (FIG. 26I). Also found was increased proliferation of CD8+ TILs, as measured by expression of cell cycle associated protein Ki67 (from 12.3 to 22.5%, p<0.05) (FIG. 26J). Next, the expression of an array of immune checkpoints markers in the TILs, including PD-1, TIM3, Ox40, CTLA4, and LAG3 was examined. Treatment with aPD-1 resulted in an expected decrease in PD-1 expression, but LB-100 alone or in addition to aPD-1 did not further alter PD-1 expression in both CD4+ and CD8+ TILs (FIG. 26K-L). Expression of TIM3, Ox40, CTLA4 and LAG3 were not significantly changed in CD4+ or CD8+ TILs with single or combination treatment (FIG. 32), suggesting that there is potential in combining LB-100 with targeted therapeutics against these check point markers.

Given the previous study demonstrating that PP2A serve an essential role in suppressive Treg, it was then examined whether addition of LB-100 could result in Treg depletion, similar to anti-CTLA4 therapy. aPD-1 is known to act at the level of the tumor and with limited ability to deplete Tregs. However, with addition of LB-100, the combination treatment significantly decreased the percentage of CD4+FoxP3+Treg cells among TILs (from 10.3 to 4.9% of CD3+ T cells, p<0.05) (FIG. 27A-B). The concomitant decrease in Treg and increase in CD8+ T cells resulted in a dramatic increase in the CD8+ to Treg ratio by 3.5-fold among the TILs (from 7.5 to 26.4, p<0.05) (FIG. 27C). Subsequently, the functional consequence of LB-100/aPD-1 combination in TILs was assessed. Intracellular expression of IFN-γ in response to in vitro stimulation with PMA/ionomycin was analyzed. Combination treatment significantly enhanced IFN-γ production by CD8+ TILs relative to control (from 16.6 to 31.5% of CD45+, p<0.05) (FIG. 27D-E). In addition, the frequency of tumor necrosis factor alpha (TNF-α)-producing (FIGS. 27F, 33A) and IFN-γ/TNF-α dual producing (FIGS. 27G, 33B) CD8+ TILs were significantly increased with the combination treatment. The cytolytic capacity of CD8+ TILs was also determined by Granzyme B (GzmB) expression, which was also significantly increased with LB-100/a-PD1 treatment (FIGS. 27H, 33C). In CD4+ T cells a small, but statistically significant increase in IFN-γ production (from 6.1 to 10.8% of CD4+ cells, p<0.05) was observed (FIG. 27I). This suggests that while there is no overall increase in CD4+ infiltration with LB-100/a-PD1 combination, effector CD4+ T cells present in the tumor were nonetheless more functionally active with enhanced IFN-γ production.

Taken together, combining LB-100 with aPD-1 blockade resulted in a significant change in the composition of TILs (FIG. 27J). While the overall CD45+ population remained relatively stable, there was a marked increase in CD3+ T cell infiltration, driven by a preponderance of CD8+ T cells. At the same time, the Treg population was concomitantly depleted resulting in a dramatic increase in CD8/Treg ratio. In addition, CD8+ T cells were more proliferative and functionally active as indicated by cytokine expressions. These findings are consistent with the observation that LB-100/a-PD1 combination could elicit durable tumor rejection in CT26 in an immune-dependent manner.

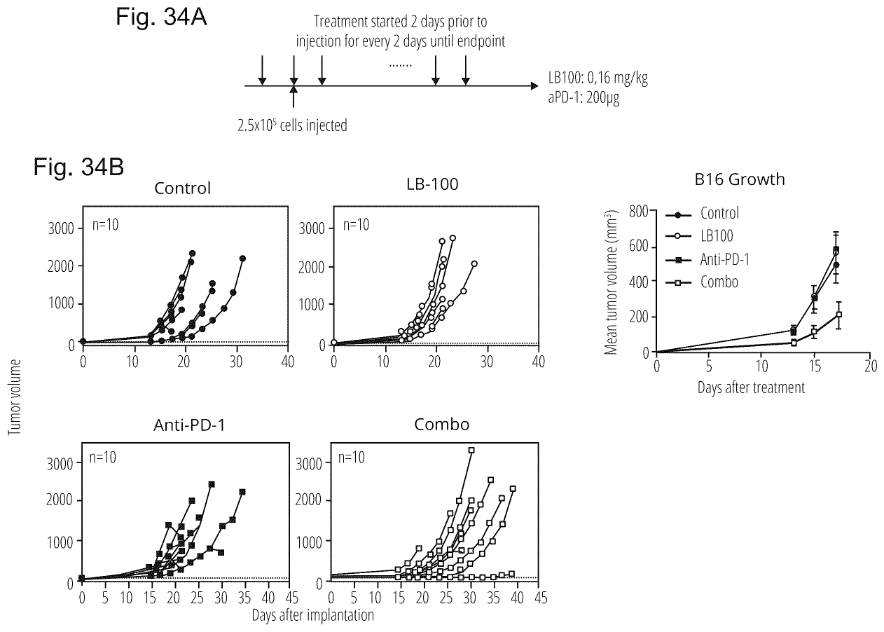

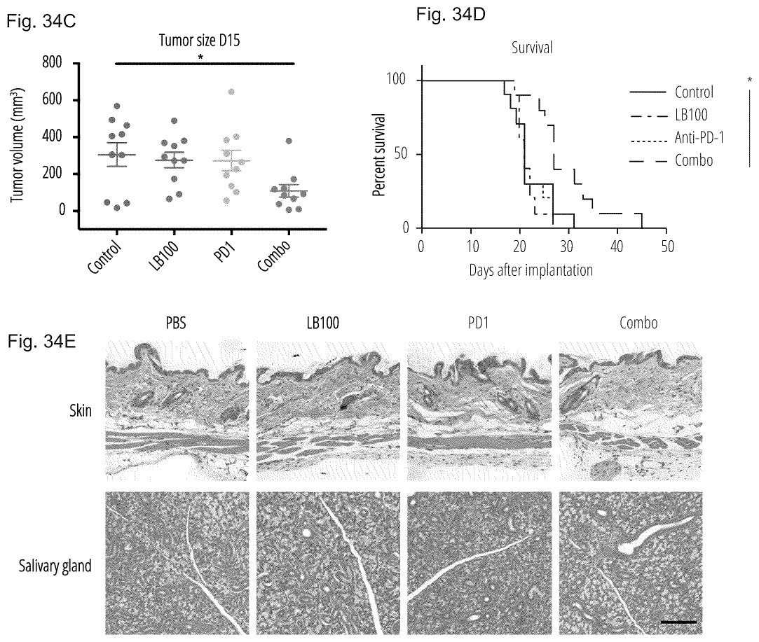

LB-100 and aPD-1 enhance anti-tumor activity in B16 melanoma without histologic evidence of autoimmunity: It was next determined whether LB-100/aPD-1 combination was effective against other aPD-1 resistant tumor. In a tumor prevention model, 6-8 weeks old C57BL/6 mice were randomized into four treatment groups: PBS, LB-100, aPD-1 and combination. B16F10 cells (2.5×10⁵) were inoculated 2 days after initiation of treatment subcutaneously in the right thoracic flank. Treatments were administered every two days following survival (FIG. 34A). By day 15 after tumor implantation, there was no difference between control and the single agent arms. However, tumor size was significantly smaller in the combination group relative to control (from 305.9 to 109.0 mm³, p<0.05) (FIG. 34B-C) and survival was prolonged by the combination treatment (p<0.05) (FIG. 34D).

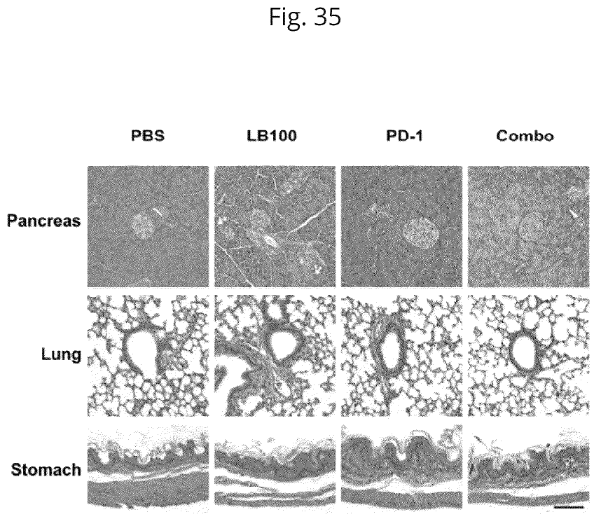

It is noteworthy that none of the mice in the combination group demonstrated any clinical signs of autoimmune inflammatory events. However, given that the LB-100/aPD-1 combination resulted in increased effector function and Treg depletion, autoimmunity is a concern. We, therefore, examined the histology of multiple organs of treated mice to look for signs of inflammation. C57BL/6 mice that reached survival endpoints were sacrificed and the histology of the skin, salivary gland, pancreas, lung and stomach were examined (FIGS. 34E, 35). There was no evidence in any of the treatment group to suggest increased lymphocyte infiltration or signs of autoimmunity.

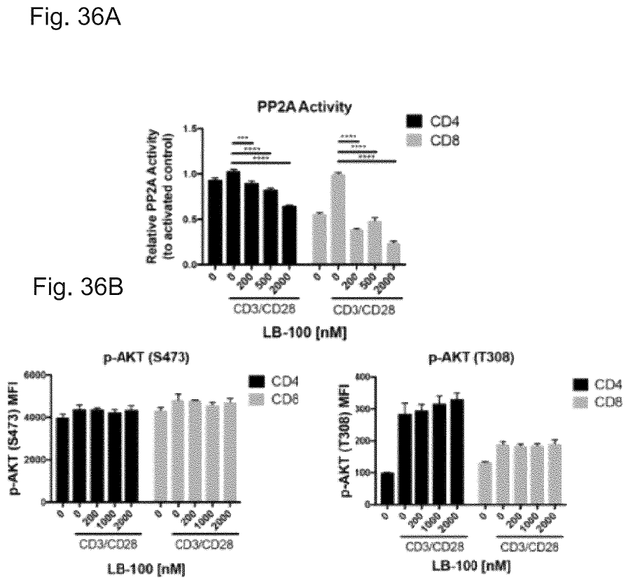

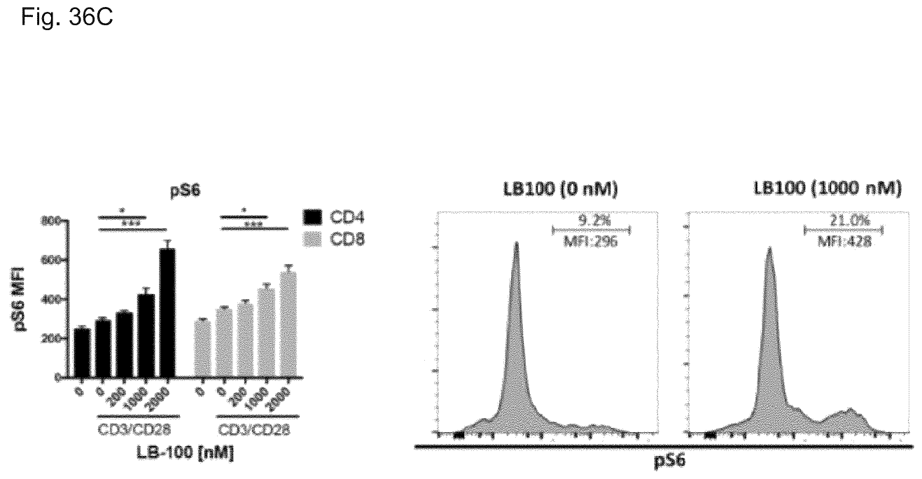

LB-100 inhibits PP2A activity and enhances mTORC1 activation: PP2A enzymatic activity of isolated CD4 and CD8 cells from mice splenocytes were measured 3 hours after in vitro stimulation with plated CD3 and soluble CD28. There was a dose dependent decrease in PP2A enzymatic activity in both CD4 and CD8 cells, with a greater effect in CD8 than CD4 cells (FIG. 36A).

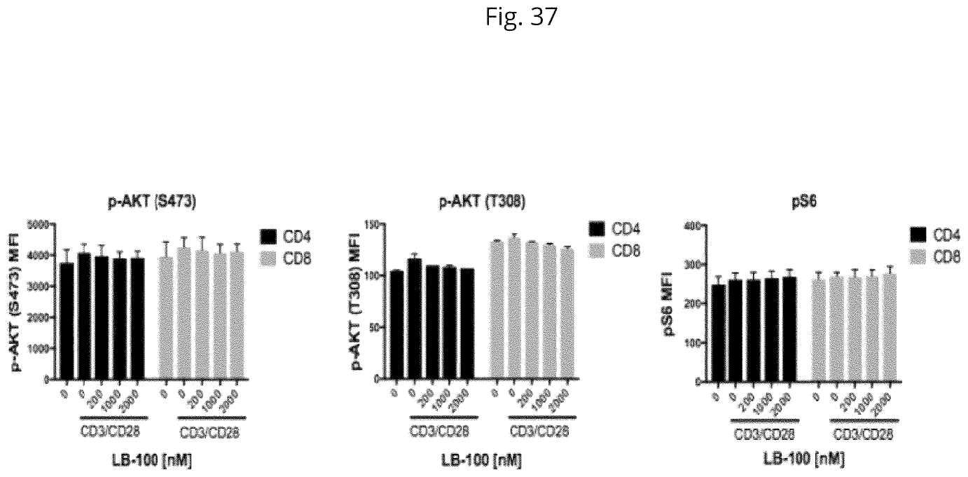

After 3 hours of in vitro activation of isolated CD3 cells, the activity of mTORC1, mTORC2 and PI(3)K-AKT pathways were assessed by checking the phosphorylation of ribosomal S6 protein (S6), AKT at Thr473 and AKT at Thr308 respectively. It was found that while LB-100 has minimal effect on mTORC2 and PI(3)K-AKT pathways (FIG. 36B), there was a dose dependent increase in activity of mTORC1 as measured by phosphorylation of S6 (FIG. 36c). This difference was not observed in any of the 3 pathways at an early time point of 30 minutes after activation (FIG. 37).

LB-100 Inhibits Naïve CD4 Cells Development into Regulatory or Th2 CD4 Cells:

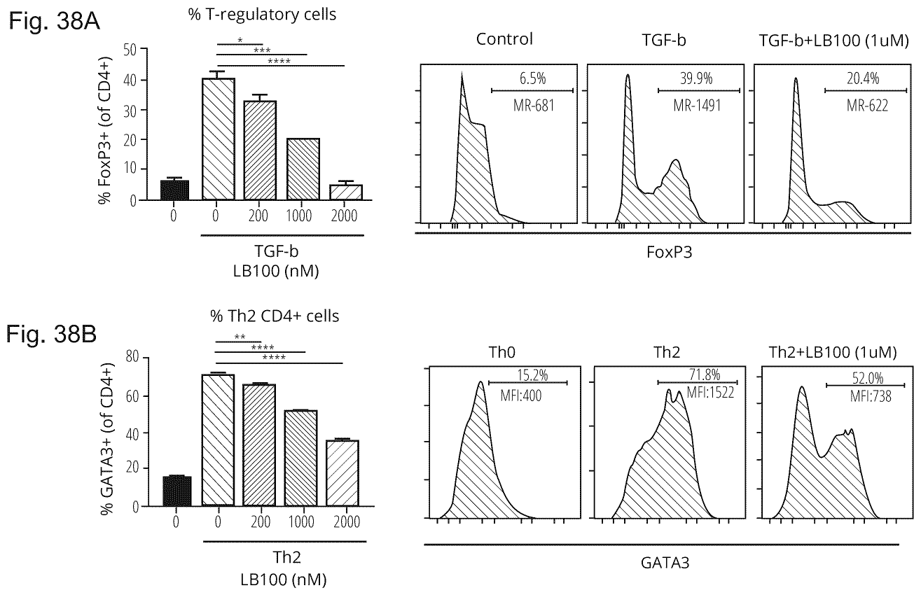

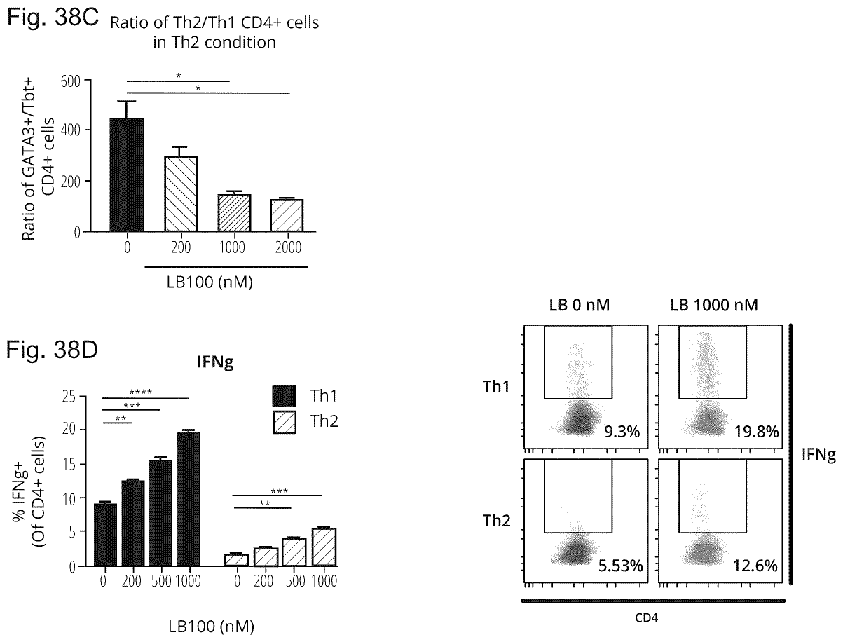

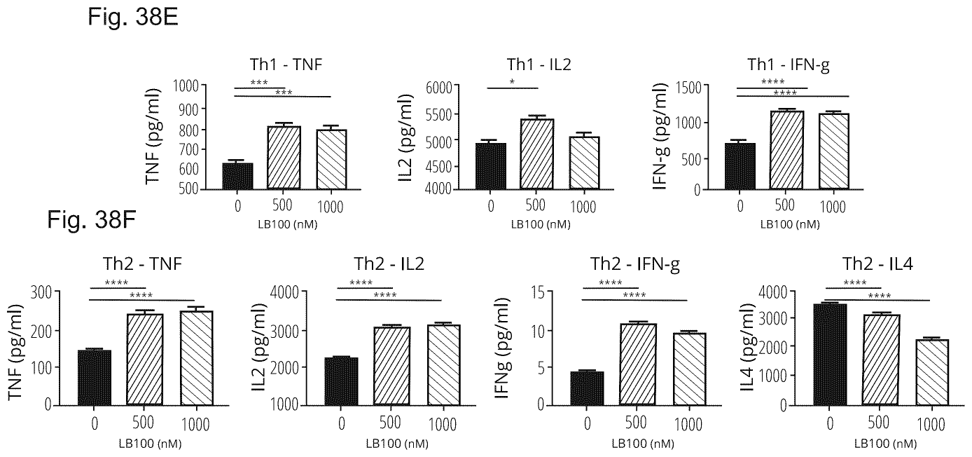

Naïve CD4 cells were isolated from mice splenocytes and activated in vitro with anti-CD3 and CD28 in the presence of TGF-β or IL4 to induce development of Treg or Th2 CD4+ cells respectively. After 72 hours, intranuclear expressions of Foxp3 or GATA3 were quantitated by flow cytometry to determine percentage of Treg or Th2 cells respectively. LB-100 treatment significantly impaired induction of Foxp3 by TGF-β (FIG. 38A) or GATA3 (FIG. 38B) by IL-4 in a dose dependent manner. In addition, the relative proportion of Th2 and Th1 CD4+ cells was quantified by labeling for T-bet. The frequency of GATA3 expressing cells relative to Tbet expressing cells decreased significantly with LB-100 treatment (FIG. 38C). Next, the functional consequence of Th1 CD4+ cell with LB-100 treatment was explored. Under both Th1 and Th2 skewing conditions, there was a dose dependent increase in IFN-γ expression with PP2A inhibition. This was shown with both intracellular staining (FIG. 38D) and measurement of cytokine secretion (FIG. 38E-F). Other Th1-related cytokines, including TNF-α and IL2 were also increased in both Th1 and Th2 conditions. Secretion of IL4 was expectedly decreased (FIG. 38F). These data suggest that PP2A inhibition decreased Treg formation and skewed CD4 cells differentiation towards Th1 lineage resulting in an overall increase in Th1 cytokine secretion. These in vitro experiments are consistent with the in-vivo TILS findings and potentially suggest that PP2A inhibition enhanced cancer immunity via mTORC1 hyper activation

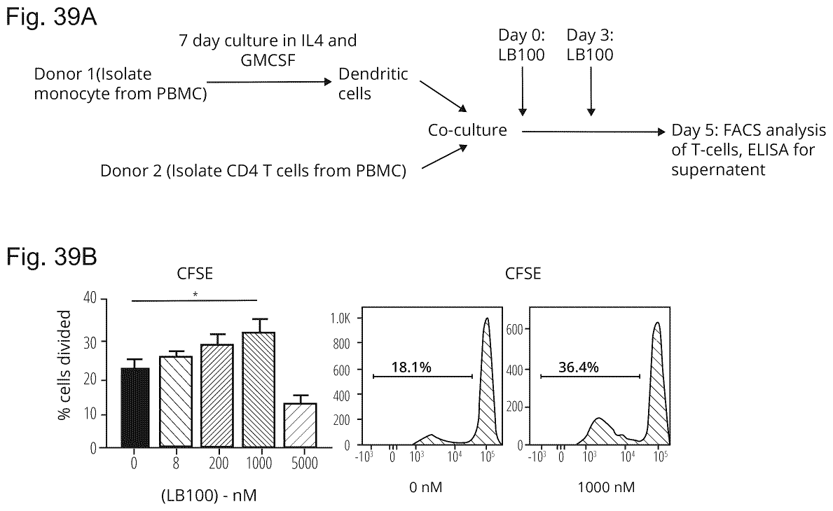

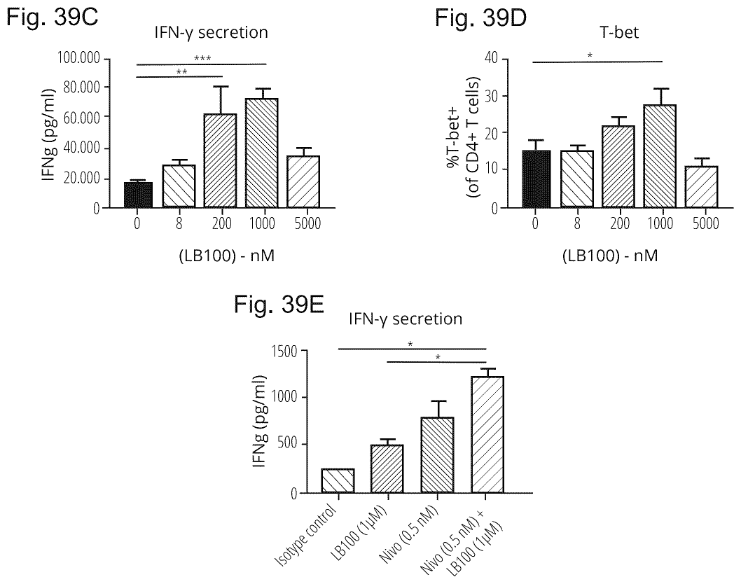

In vitro activity of LB-100 in human mixed lymphocytes reactions: To further confirm the immune-modulating effect of LB-100 has clinical utility, mixed lymphocyte reactions (MLRs) were performed using PBMC from healthy human donors. Monocyte derived dendritic cells were co-cultured with allogenic CD4+ T cells labelled with cytosolic dye CFSE. LB-100 was given on the day of co-culture (Day 0) and again on Day 3. Proliferation and IFN-γ secretion by CD4 T cells were assessed on Day 5 (FIG. 39A). There was a significant increase in CD4 T cell proliferation, as measured by the percentage of dividing cells, with LB-100 treatment at 1 M (31% compared to 20% in controls) (FIG. 39B). There was also a trend towards increased proliferation at lower LB100 concentrations (in the sub-micromolar range). At the high dose of 5 μM, proliferation was impaired suggesting that there is an optimal window of LB-100 exposure that enhances immunity. A similar pattern was observed with IFN-γ secretion (FIG. 39C). At 0.2 and 1 M of LB-100, IFN-γ release was significantly enhanced 3.5 to 4-fold respectively. Also examined was the effect of lineage differentiation in CD4 T cells by labeling for T-bet. LB-100 at 1 M significantly increased T-bet expression (FIG. 39D), confirming our previous finding that LB-100 appears to skew CD4 lineage towards Th1 differentiation. Next tested whether LB-100 could enhance IFN-γ secretion in vitro in combination with PD1 blockade using Nivolumab. A similar MLR assay was performed with LB-100. It was found that LB-100 synergized with anti-PD1 (aPD-1) blockade and enhanced IFN-γ secretion compared to single agents (FIG. 39E).

### Materials and Methods

Drugs—Nivolumab was obtained from Bristol-Myers Squibb and LB-100 was obtained from Lixte Biotechnology Holdings, Inc.

Cell lines—CT26.CL25 colon carcinoma, B16 F10 melanoma and 4T1 mammary carcinoma cell lines were obtained from ATCC. Tumor cells were cultured in complete medium (RPMI 1640, Gibco) containing 10% (vol/vol) FBS (Gibco), 100 U/ml penicillin, 100 μg/ml streptomycin (Gibco).

Syngeneic tumor models—Mice were maintained and experiments were conducted with the approval of the NINDS Animal Use and Care Committees. For CT26 tumors: BALB/c (6-8 wk old) were purchased from Charles River Laboratory. CT26 cells (0.5×10⁶) were injected into the right flank subcutaneously. Once tumors reached a volume of 30-100 mm³ (day 0), mice were randomized and treated with PBS, LB-100 (0.156 mg/kg) and/or anti-mouse PD-1 (10 mg/kg) (RMP1-14; rat IgG2b; Bio X Cell). Treatments were given every 2 days for 30 days. Tumor volume was measured every 2 days using a caliper and tumor volume was calculated according to the formula: Volume (mm³)=L×W²/2, where L is the length and W is the width of the tumor (in millimeters. For B16 tumors: C57BL/6 (6-8 wk old) were purchased from Charles River Laboratory. Mice were randomized into respective treatment groups and 2 days after initial treatment B16F10 cells (0.5×10⁶) were injected into the right flank subcutaneously. Treatment and measurements were done every 2 days. Survival end-point was defined as when any of the following criteria was reached: 1) tumor volume exceeding 2000 mm³, 2) tumor diameter exceeding 2 cm, 3) severe non-healing skin necrosis over the tumor. When indicated, some mice were depleted for CD8+ T cells by injection of 250 ∝g of CD8-depleting antibodies (clone 53.6.7; BioXcell). Injections were given 2 d and 1 d before therapy, on the day of therapy initiation, at 5 d and 8 d after start of therapy, and weekly onwards.

Tumor re-challenge studies—Naïve BALB/c mice and previously cured (CR) mice with combination therapy from CT26 tumors were inoculated with 0.5×10⁶ CT26 cells into the left (CR) and right (naïve) thoracic flank. Where indicated, some mice were also inoculated with 1.25×10⁵ 4T1 mammillary carcinoma cells in the mammary fat pad. Tumors volume were then monitored similarly as above.

Isolation of TILs—Mice were injected in the right thoracic flank with 0.5×10⁶ CT26 cells and treated as above after tumors reached between 50-100 mm³. After 2 treatments, mice were sacrificed and tumors excised. Tumors were subjected to mechanical disruption using a GentleMACS Dissociator (Miltenyi Biotec) in presence of enzymatic digestion using Tumor Dissociation Kit (Miltenyi Biotec). Gating strategy used for analysis of TILs is shown in Fig. S3. Intracellular cytokine staining, phosphoflow and flow cytometry—Suspensions containing T cells were stained with a fixable live/dead stain (Invitrogen) in PBS followed by surface antibody staining in FACS buffer (PBS with 0.5% BSA and 0.1% sodium azide). For intracellular staining, cells were stained for surface molecules following by fixation and permeabilization (eBioscience). For cytokine staining, cells were first stimulated with Cell Stimulation Cocktail (eBioscience) containing PMA/Ionomycin and protein transport inhibitor prior to undergoing staining. For phosphostaining, 4% formaldehyde was used for fixation and 100% methanol was used for permeabilization protocols. Cells were analyzed by flow cytometry (LSRII; BD Bioscience). Data analysis was performed using FlowJo software (TreeStar).

PP2A phosphatase assay—Mouse CD4+ and CD8+ T cells were isolated with CD4 and CD8 isolation kit (StemCell) respectively. Cells were activated using immobilized anti-CD3 (10 ug/ml) and soluble anti-CD28 (2 ug/ml) for three hour. PP2A activity was then evaluated after immunoprecipitation using a malachite green phosphatase assay kit as per the manufacturer's instructions (EMD Millipore).

T cell stimulation and skewing—Naïve CD4 cells were isolated from mice splenocytes (StemCell). Cells were activated for 3 days using immobilized anti-CD3 (10 ug/ml) and soluble anti-CD28 (2 ug/ml). Skewing conditions were as follows: T_{H}1, 1.ig/mL anti-IL4, 5 ng/mL IL2, and 10 ng/mL IL12; T_{H}2, 1.ig/mL anti-IFN-γ, 5 ng/mL IL2, and 10 ng/mL IL4; Treg, 1.ig/mL anti-IFNγ, and 1 ig/mL anti-IL4, and 2 ng/mL TGF(31. Bead-based multianalyte flow immunoassays (BD Bioscience) were used to measure cytokine production in the supernatant per manufacturer's instruction. Absolute cell numbers were quantified with flow cytometry using counting beads (Biolegend).

Antibodies for flow cytometry—Anti-mouse: α-CD45 (30-F11, BD), α-CD3 (145-2C11, Biolegend), α-CD4 (GKL15, Biolegend), α-CD8 (53-6.7, BD), α-PD-1 (J43, ThermoFisher), α-CTLA4 (1B8, abeam), α-TIM-3 (B8.2C12, Biolegend), α-OX-40 (OX-86, Biolegend), α-CD62L (MEL-14, BD), α-CD44 (IM7, Biolegend), α-LAG-3 (C9B7W, Biolegend), α-IFN-γ (XMG1.2, Biolegend), α-TNF-α (MP6-XT22, Biolegend), α-Granzyme B (NGZB, ThermoFisher), α-FOXP3 (MF-14, Biolegend), α-Ki67 (SolA15, ThermoFisher). Anti-human: α-CD4 (A161A1, Biolegend), α-T-bet (4B10, Biolegend), α-Phospho-Akt (Ser473) (D9E, Cell Signaling), α-Phospho-Akt (Thr308) (D25E6, Cell Signaling), α-Phospho-S6 Ribosomal Protein (Ser235/236) (D57.2.2E, Cell Signaling).

Histology—Formalin-fixed tissues were processed, stained with hematoxylin and eosin and evaluated blindly by a board-certified pathologist.

Human mixed lymphocyte reaction—as previously described in 39. Dendritic cells (DCs) were generated by culturing monocytes isolated from PBMC using a monocyte isolation kit (StemCell) in vitro for 7 days with 500 U/ml interleukin-4 (IL-4) and 250 U/ml GM-CSF (R&D Systems). CD4+ T cells (1×10⁵) isolated with CD4 isolation kit (StemCell) and labeled with CFSE (ThermoFisher) were co-cultured with allogeneic DCs (1×10⁴). At the initiation of assay, a titration of LB-100 and/or Nivolumab was added. After 3 days, LB-100 was replenished to the final indicated concentration. After 5 days, culture supernatants were analyzed by ELISA (eBioscience) and cells were analyzed by flow cytometry. At least 3 separate donors were obtained and results of one representative donor were reported.

Statistics—If not stated otherwise in the figure legend, samples were analyzed with GraphPad Prism software using Tukey's multiple comparison test. Scatter dot plots are depicted as means with SEM.

### REFERENCES

Apostolidis S, Rodríguez-Rodríguez N, Suárez-Fueyo A et al. Phosphatase PP2A is requisite for the function of regulatory T cells. Nat Immunol. 2016, 17:556-64.
Bai X, Zhi X, Zhang Q et al. Inhibition of protein phosphatase 2A sensitizes pancreatic cancer to chemotherapy by increasing drug perfusion via HIF-1α-VEGF mediated angiogenesis. Cancer Lett. 2014, 355:281-287.
Bai X L, Zhang Q, Ye L Y, et al. Inhibition of protein phosphatase 2A enhances cytotoxicity and accessibility of chemotherapeutic drugs to hepatocellular carcinomas. Mol Cancer Ther. 2014, 13:2062-72.
Baroja, M. L. et al. Inhibition of CTLA-4 Function by the Regulatory Subunit of Serine/Threonine Phosphatase 2A. J Immunol 2002; 168:5070-5078.
Bertini I, Calderone V, Fragai M, Luchinat C, Tallu E. Structural Basis of Serine/Threonine Phosphatase Inhibition by the Archetypal Small Molecules Cantharidin and Norcantharidin J. Med. Chem. 2009, 52:4838-4843.
Bian Y, Kitagawa R, KB Parmil, Fujii Y, Stepanov A, and Kitagawa K. Synthetic genetic array screen identifies PP2A as a therapeutic target in Mad2-overexpressing tumors. Proc Natl Acad Sci USA 2014, 111:1628-1633
Brahmer J R, et al. Phase I study of single-agent anti-programmed death-1 (MDX-1106) in refractory solid tumors: safety, clinical activity, pharmacodynamics, and immunologic correlates. J Clin Oncol 28, 31673175 (2010).
Brunet J F, Denizot F, Luciani M F, Roux-Dosseto M, Suzan M, Mattei M G, Golstein P: A new member of the immunoglobulin superfamily—CTLA-4. Nature 1987, 328:267-270.
Carreno B M, Bennett F, Chau T A, Ling V, Luxenberg D, Jussif J, Baroja M L, Madrenas J: CTLA-4 (CD152) can inhibit T cell activation by two different mechanisms depending on its level of cell surface expression. J Immunol 2000, 165:1352-1356.
Chang K E, Wei B R, Madigan J P et al. The Protein Phosphatase 2A Inhibitor LB-100 Sensitizes Ovarian Carcinoma Cells to Cisplatin-Mediated Cytotoxicity. Mol Cancer Ther. 2015, 14:90-100.
Chapman N M, Chi H. mTOR signaling, Tregs and immune modulation. Immunotherapy 6, 1295-1311 (2014).
Chen D S, Mellman I. Oncology meets immunology: the cancer-immunity cycle. Immunity 39, 1-10 (2013).
Chuang, E. et al. The CD28 and CTLA-4 Receptors Associate with the Serine/Threonine Phosphatase PP2A. 2000, Immunity, Vol. 13, 313-322.
Chung V, Mansfield A S, Braiteh F, Richards D, Durivage H, Ungerleider R S, Johnson F, and Kovach J S. Safety, tolerability, and preliminary activity of LB-100, an inhibitor of protein phosphatase 2A, in patients with relapsed solid tumors. Clin Cancer Res 2017.
Chung, V. Phase I study of LB-100 with docetaxel in solid tumors. 2013: ClinicalTrials.gov.
Delgoffe G M, et al. The kinase mTOR regulates the differentiation of helper T cells through the selective activation of signaling by mTORC1 and mTORC2. Nat Immunol 12, 295-303 (2011).
Delgoffe G M. PP2A's restraint of mTOR is critical for T(reg) cell activity. Nat Immunol 17, 478-479 (2016).
Ebert P J, et al. MAP Kinase Inhibition Promotes T Cell and Anti-tumor Activity in Combination with PD-L1 Checkpoint Blockade. Immunity 44, 609-621 (2016).
Efferth, T. et al. (2005) Molecular modes of action of cantharidin in tumor cells. Biochem Pharmacol. 69(5): p. 811-8.
Efferth, T. et al. (2002) Activity of drugs from traditional Chinese medicine toward sensitive and MDR1- or MRP1-overexpressing multidrug-resistant human CCRF-CEM leukemia cells. Blood Cells Mol Dis. 28(2): p. 160-8.
Eil R, Vodnala S K, Clever D., Klebanoff C A, Sukumar M, Pan J H, Palmar D C, Gros A, Yamamoto T N, Patel S J, Guittard G C, Yu Z, Carbonaro V, Okkenhaug K, Schrump D S, Linehan W M, Roychoudhuri R, Restifo N P. Ionic immune suppression within the tumour microenvironment limits T cell effector function. Nature 2016, 537:539-543.
Falconer I R, Humpage A R. Preliminary evidence for in vivo tumour initiation by oral administration of extracts of the blue-green alga cylindrospermopsis raciborskii containing the toxin cylindrospermopsin. Environ Toxicol 16, 192-195 (2001).
Gehringer M M. Microcystin L R and okadaic acid-induced cellular effects: a dualistic response. FEBS Lett 557, 1-8 (2004).
Gordon I K, Lu J, Graves C A et al. Protein Phosphatase 2A Inhibition with LB-100 Enhances Radiation-Induced Mitotic Catastrophe and Tumor Growth Delay in Glioblastoma. Mol Cancer Ther. 2015, 14:1540-47.
Haxhinasto S, Mathis D, Benoist C. The AKT-mTOR axis regulates de novo differentiation of CD4+Foxp3+ cells. J Exp Med 205, 565-574 (2008).
Hein A L, Seshacharyulu P, Rachagani S et al. PR55a Subunit of Protein Phosphatase 2A Supports the Tumorigenic and Metastatic Potential of Pancreatic Cancer Cells by Sustaining Hyperactive Oncogenic Signaling. Cancer Res. 2016, 8:2243-53
Ho W S, Feldman M J, Maric D et al. PP2A inhibition with LB-100 enhances cisplatin cytotoxicity and overcomes cisplatin resistance in medulloblastoma cells. Oncotarget. 2016, 7:12447-63.
Hodi, F. S. et al. Improved Survival with Ipilimumab in Patients with Metastatic Melanoma. 2010, N Engl J Med, 363; 8.
Holmgaard R B, Zamarin D, Munn D H, Wolchok J D, Allison J P. Indoleamine 2,3-dioxygenase is a critical resistance mechanism in antitumor T cell immunotherapy targeting CTLA-4. J Exp Med 210, 1389-1402 (2013).
Hong C S, Ho W, Zhang C, Yang C, Elder J B, Zhuang Z. LB-100, a Small Molecule Inhibitor of PP2A with Potent Chemo- and Radio-sensitizing Potential. Cancer Biol Ther. 2015, 16:821-33.
Janssens V, Longin S, Goris J. PP2A holoenzyme assembly: in cauda venenum (the sting is in the tail). Trends Biochem Sci 33, 113-121 (2008).
Janssens V, Rebollo A. The Role and Therapeutic Potential of Ser/Thr Phosphatase PP2A in Apoptotic Signalling Networks in Human Cancer Cells. Curr Mol Med. 2012, 12:268-287.
Joseph F. Grosso and Maria N. Jure-Kunkel. CTLA-4 blockade in tumor models: an overview of preclinical and translational research. Cancer Immun. 2013; 13: 5.
Kiely M, Kiely P A. PP2A: The Wolf in Sheep's Clothing?Cancers (Basel) 7, 648-669 (2015).
Kingwell K. Cancer: Live screening of immunotherapy targets. Nat Rev Drug Discov 13, 258 (2014).
Kovach J S, Johnson F. (2008) Oxabicycloheptanes and oxabicycloheptenes, their preparation and use. U.S. Pat. No. 7,998,957, Aug. 16, 2011.
Larkin J, et al. Combined Nivolumab and Ipilimumab or Monotherapy in Untreated Melanoma. N Engl J Med 373, 23-34 (2015).
Lê L, Erlichman C, Pillon L et al. Phase I and pharmacokinetic study of fostriecin given as an intravenous bolus daily for five consecutive days. Invest New Drugs 2004, 22:159-167.
Lecca S, et al. Rescue of GABAB and GIRK function in the lateral habenula by protein phosphatase 2A inhibition ameliorates depression-like phenotypes in mice. Nat Med 22, 254-261 (2016).
Lu, J. et al. Inhibition of serine/threonine phosphatase PP2A enhances cancer chemotherapy by blocking DNA damage induced defense mechanisms. Proc Natl Acad Sci USA, 2009. 106(28): p. 11697-702.
Lv Peng, Wang Y, Ma J et al. Inhibition of protein phosphatase 2A with a small molecule LB-100 radiosensitizes nasopharyngeal carcinoma xenografts by inducing mitotic catastrophe and blocking DNA damage repair. Oncotarget. 2014, 5:7512-7524.
Martiniova, L. et al. Pharmacologic modulation of serine/threonine phosphorylation highly sensitizes PHEO in a MPC cell and mouse model to conventional chemotherapy. PLoS One, 2011. 6(2): p. e14678.
Melero I, Rouzaut A, Motz G T, Coukos G. T-cell and NK-cell infiltration into solid tumors: a key limiting factor for efficacious cancer immunotherapy. Cancer Discov 4, 522-526 (2014).
Mumby M. PP2A: unveiling a reluctant tumor suppressor. Cell 2007; 130:21-4.
Ngiow S F, et al. A Threshold Level of Intratumor CD8+ T-cell PD1 Expression Dictates Therapeutic Response to Anti-PD1. Cancer Res 75, 3800-3811 (2015).
Parry, R. V. et al. CTLA-4 and PD-1 Receptors Inhibit T-Cell Activation by Distinct Mechanisms. Molecular and Cellular Biology, 2005, p. 9543-9553 Vol. 25, No. 21.
Perotti D, Neviani P. Targeting A Tumor Suppressor To Suppress Tumor Growth: News and Views on Protein Phosphatase 2A (PP2A) as a Target for Anti-cancer Therapy. Lancet Oncol. 2013, 14:e229-e238.
Pico de Coana, Y. et al. Checkpoint blockade for cancer therapy: revitalizing a suppressed immune system Trends in Molecular Medicine (2015) 1-10.
Quang C, Simko J L, Nethero W C, Groeber E A, Kovach J S. LC-MS/MS Method Development and Validation for the Quantification of LB-100 and Endothall Metabolite in Biological Matrices (20). Poster M P 158, American Society for Mass Spectrometry Conference on Mass Spectrometry and Allied Topics, Jun. 6 2016, San Antonio, TX
Robert C, et al. Nivolumab in previously untreated melanoma without BRAF mutation. N Engl J Med 372, 320-330 (2015).
Rossini G P, Sgarbi N, Malaguti C. The toxic responses induced by okadaic acid involve processing of multiple caspase isoforms. Toxicon 39, 763-770 (2001).
Sagiv-Barfi I, Kohrt H E, Czerwinski D K, Ng P P, Chang B Y, Levy R. Therapeutic antitumor immunity by checkpoint blockade is enhanced by ibrutinib, an inhibitor of both BTK and ITK. Proc Natl Acad Sci USA 112, E966-972 (2015).
Sangodkar J, Farrington C C, McClinch K, Galsky M D, Kastrinsky D B, Narla G. All roads lead to PP2A: exploiting the therapeutic potential of this phosphatase. FEBS J 2016, 283:1004-24.
Sallman D, Wei S, List A et al. PP2A: the Achilles heal in MDS with 5q deletion. Front Oncol. 2014, 4:1-7.
Schvartzman J M, Pascal H G D, Sotillo R, Coker C, Benezra R. Mad2 Is a Critical Mediator of the Chromosome Instability Observed upon Rb and p53 Pathway Inhibition. Cancer Cell 2011, 19:701-714.
Seshacharyulu P, Pandey P, Datta K et al. Phosphatase: PP2A structural importance, regulation and its aberrant expression in cancer. Cancer Lett. 2013, 335:9-118.
Shi Y. Serine/Threonine Phosphatases: Mechanism through Structure. Cell 2009, 139: 468-484.
Snyder, A. et al. Genetic Basis for Clinical Response to CTLA-4 Blockade in Melanoma. 2014, N Engl J Med, 371; 23.
Sukari A, Nagasaka M, Al-Hadidi A, Lum L G. Cancer Immunology and Immunotherapy. Anticancer Res 36, 5593-5606 (2016).
Swart M, Verbrugge I, Beltman. Combination Approaches with Immune-Checkpoint Blockade in Cancer Therapy. Frontiers in Oncology 2016, 6:233.
Taffs R E, Redegeld F A, and Sitkovsky. Modulation of cytolytic T lymphocyte functions by an inhibitor of serine/threonine phosphatase, okadaic acid. Enhancement of cytolytic T lymphocyte-mediated cytotoxicity. J Immunol. 1991, 147:722-728.
Teft W A, Kirchhof M G, Madrenas J: A molecular perspective of CTLA-4 function. Annu Rev Immunol 2006, 24:65-97.
Teft W A, Kirchhof M G, Madrenas J: Structure-Function analysis of the CTLA-4 interaction with PP2A. BMC Immunology 2009, 10:23.
Topalian S L, Drake C G, Pardoll D M. Immune Checkpoint Blockage: A Common Denominator Approach to Cancer Therapy. Cancer Cell 2015, 27:450-461.
Tsiatas M, Mountzios, Curigliano. Future perspective in cancer immunotherapy. Ann Transl Med 2016, 4(14):273.
Wang C, et al. In vitro characterization of the anti-PD-1 antibody nivolumab, BMS—936558, and in vivo toxicology in non-human primates. Cancer Immunol Res 2, 846-856 (2014).
Wang G S. Medical uses of mylabris in ancient China and recent studies. J Ethnopharmacol. 1989, 2:147-62.
Wei, D. et al. Inhibition of protein phosphatase 2A radiosensitizes pancreatic cancers by modulating CDC25C/CDK1 and homologous recombination repair. Clin Cancer Res, 2013. 19(16): p. 4422-32.
Westermarck J, Hahn W C. Multiple pathways regulated by the tumor suppressor PP2A in transformation. Trends Mol Med. 2008, 14:152-160.
Yatsunami J, Komori A, Ohta T, Suganuma M, Yuspa S H, Fujiki H. Hyperphosphorylation of cytokeratins by okadaic acid class tumor promoters in primary human keratinocytes. Cancer Res 53, 992-996 (1993).
Zhang, C. et al. A synthetic cantharidin analog for the enhancement of doxorubicin suppression of stem cell-derived aggressive sarcoma. Biomaterials, 2010. 31(36): p. 9535-43.
Zhang M, Yogesha S D, Mayfield J E, Gill G N, Zhang Y. Viewing serine/threonine protein phosphatases through the eyes of drug designers. FEBS J 280, 4739-4760 (2013).
Zhou P, Shaffer D R, Alvarez D A et al. In vivo Discovery of Immunotherapy Targets in the Tumor Microenvironment. Nature 2014, 506:52-57.
Zhuang Z, Lu J, Lonser R, Kovach J S. Enhancement of cancer chemotherapy by simultaneously altering cell cycle progression and DNA-damage defenses through global modification of the serine/threonine phospho-proteome. Cell Cycle. 2009, 8:3303-6.
U.S. Pat. No. 8,697,845 B2, issued Apr. 15, 2014.

1. A method of treating a subject afflicted with cancer comprising administering to the subject an effective amount of a PP2A inhibitor in combination with an effective amount of a checkpoint inhibitor, wherein the amounts when taken together are effective to treat the subject.

2. A method of treating a subject afflicted with cancer and receiving a checkpoint inhibitor comprising administering to the subject of an amount of PP2A inhibitor effective to enhance treatment relative to the checkpoint inhibitor alone.

3. A method of treating a tumor or cancer in a subject comprising administering to the subject an effective amount of a PP2A inhibitor in combination with an effective amount of a checkpoint inhibitor, wherein the amounts when taken together are effective to treat the tumor or cancer.

4. A method of increasing a T-cell response to cancer cells in a subject afflicted with cancer comprising administering to the subject an amount of a PP2A inhibitor in combination with an effective amount of a checkpoint inhibitor effective to increase the T-cell response to the cancer cells.

5. A method of increasing T cell activation in a subject afflicted with cancer comprising administering to the subject an effective amount of a PP2A inhibitor in combination with an effective amount of a checkpoint inhibitor so as to thereby increase the T cell activation.

6. The method of any one of claims 1-5, wherein the amount of the compound and the amount of the checkpoint inhibitor are each periodically administered to the subject.

7. The method of any one of claims 1-6, wherein the amount of the compound and the amount of the checkpoint inhibitor are administered simultaneously, separately or sequentially.

8. The method of any one of claims 1-7, wherein the checkpoint inhibitor is administered concurrently with, prior to, or after the PP2A inhibitor.

9. The method of any one of claims 1-8, wherein the amount of checkpoint inhibitor and the amount of compound when administered together is more effective to treat the subject than when each agent at the same amount is administered alone.

10. The method of any one of claims 1-9, wherein the amount of the compound and the amount of the checkpoint inhibitor when taken together is effective to reduce a clinical symptom of the cancer in the subject.

11. The method of any one of claims 1-10, wherein the compound enhances the immunotherapeutic effect of the checkpoint inhibitor.

12. The method of any one of claims 1-11, wherein the cancer is susceptible to treatment by an immune response.

13. The method of any one of claims 1-12, wherein the immune checkpoint inhibitor is a CTLA-4 agent.

14. The method of claim 13, wherein the CTLA-4 checkpoint inhibitor is ipilimumab or tremelimumab.

15. The method of any one of claims 1-12, wherein the immune checkpoint inhibitor is an Anti-PD-1 or Anti-PD-L1 agent.

16. The method of claim 15, wherein the PD-1 and/or PD-L1 checkpoint inhibitor is atezolizumab, nivolumab or pembrolizumab.

17. The method of any one of claims 1-16, wherein the cancer is melanoma, renal cell carcinoma, prostate cancer, urothelial carcinoma or ovarian cancer.

18. The method of claim 17, wherein the cancer is melanoma.

19. The method of any one of claims 1-16, wherein the compound is administered at a dose of 0.25 mg/m², 0.5 mg/m², 0.83 mg/m², 1.25 mg/m², 1.75 mg/m², 2.33 mg/m², of 3.1 mg/m².

20. The method of claim 19, wherein the compound is administered at a dose of 2.33 mg/m².

21. The method of any one of claims 1-16, wherein the compound is administered for 3 days every 3 weeks.

22. The method of claim 14, wherein the ipilimumab is administered intravenously at a dose of 0.5 mg/kg-10 mg/kg or less.

23. The method of claim 22, wherein the ipilimumab is administered intravenously over 90 minutes every 3 weeks or less.

24. The method of claim 14, wherein the atezolizumab is administered intravenously at a dose of 0.1 mg/kg-20 mg/kg or less.

25. The method of claim 22, wherein the atezolizumab is administered intravenously over 60 minutes every 3 weeks or less.

26. The method of claim 16, wherein the nivolumab is administered intravenously at a dose of 0.1 mg/kg-10 mg/kg or less.

27. The method of claim 26, wherein the nivolumab is administered intravenously over 60 minutes every 2 weeks or less.

28. The method of claim 16, wherein the pembrolizumab is administered intravenously at a dose of 1 mg/kg-10 mg/kg or less.

29. The method of claim 28, wherein the pembrolizumab is administered intravenously over 30 minutes every 3 weeks or less.

30. A method of inhibiting the function of a CTLA-4 in T cells comprising administering to the T cells a PP2A inhibitor so as to thereby inhibit the function of the CTLA-4.

31. A method of inhibiting the PD-1:PD-L1 interaction in T cells comprising administering to the T cells a PP2A inhibitor so as to thereby inhibit the interaction of PD-1:PD-L1.

32. The method of any one of claims 1-23, wherein the PP2A inhibitor has the structure: 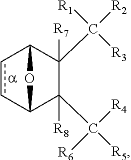
wherein
bond α is present or absent;
R₁ and R₂ together are ═O;
R₃ is OH, O—, OR₉, O(CH₂)₁₋₆R₉, SH, S—, or SR₉,
wherein R₉ is H, alkyl, alkenyl, alkynyl or aryl;
R₄ is 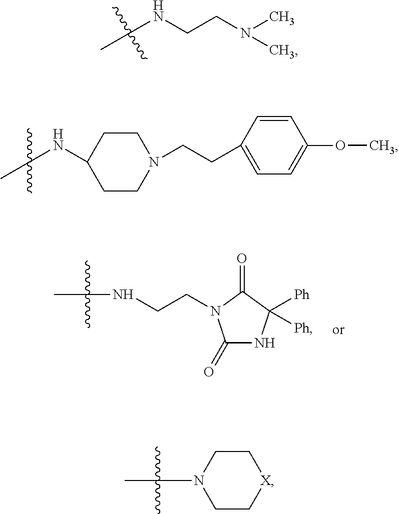
where X is O, S, NR₁₀, N⁺HR₁₀ or N⁺R₁₀R₁₀,
where each R₁₀ is independently H, alkyl, alkenyl, alkynyl, aryl, 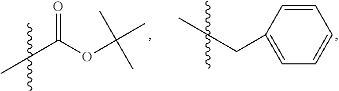
—CH₂CN, —CH₂CO₂R₁₁, or —CH₂COR₁₁,
wherein each R₁ is independently H, alkyl, alkenyl or alkynyl;
R₅ and R₆ taken together are ═O;
R₇ and R₈ are each H,
or a salt, zwitterion, or ester thereof.

33. The method of claim 32, wherein the compound has the structure:

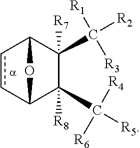

34. The method of claim 32 or 33, wherein bond α in the compound is present.

35. The method of claim 32 or 33, wherein bond α in the compound is absent.

36. The method of claim 32 or 33, wherein
R₃ is OH, O—, or OR₉,
wherein R₉ is alkyl, alkenyl, alkynyl or aryl;
R₄ is 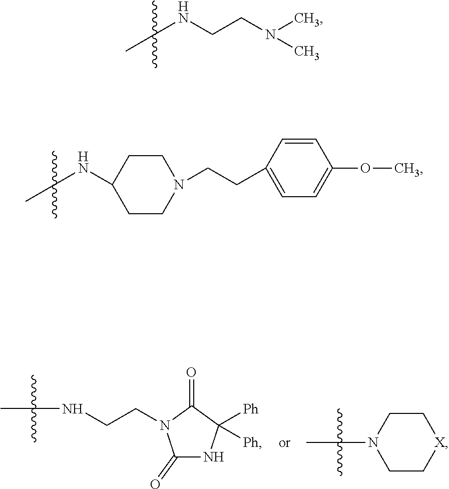
where X is O, S, NR₁₀, N⁺HR₁₀ or N⁺R₁₀R₁₀,
where each R₁₀ is independently H, alkyl, alkenyl, alkynyl, aryl,

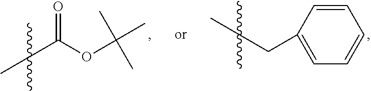

37. The method of claim 36, wherein R₃ is OH, O— or OR₉, where R₉ is H, methyl, ethyl or phenyl.

38. The method of claim 37, wherein R₃ is OH, O— or OR₉, wherein R₉ is methyl.

39. The method of claim 36, wherein R₄ is

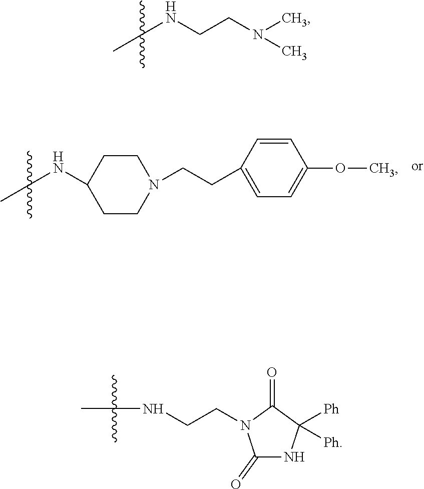

40. The method of claim 36, wherein R₄ is 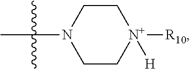
wherein R₁₀ is H, alkyl, alkenyl, alkynyl, aryl, or

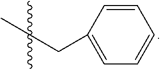 [SMILES: *Cc1ccccc1]

41. The method of claim 40, wherein R₄ is H 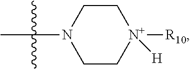
wherein R₁₀ is —H, —CH₃, —CH₂CH₃, or

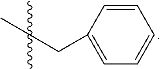 [SMILES: *Cc1ccccc1]

42. The method of claim 41, wherein R₄ is

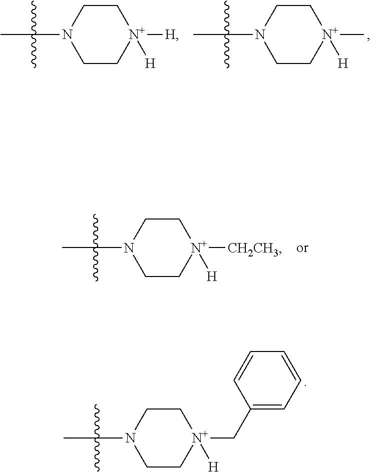

43. The method of claim 36, wherein R₄ is 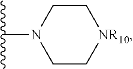
wherein R₁₀ is H, alkyl, alkenyl, alkynyl, aryl,

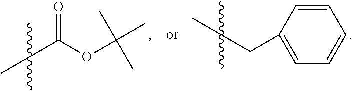

44. The method of claim 43, wherein R₄ is

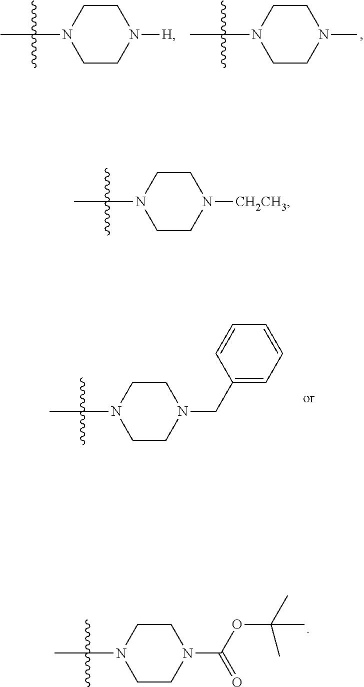

45. The method of claim 36, wherein R₄ is

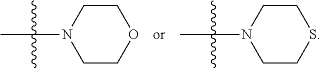 [SMILES: *N1CCOCC1 ; *N1CCSCC1]

46. The method of claim 34 or 35, wherein the compound has the structure 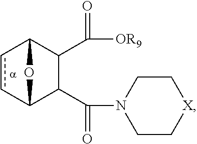
wherein:
bond α is present or absent;
R₉ is present or absent and when present is H, alkyl, alkenyl, alkynyl or phenyl; and
X is O, NR₁₀, NH⁺R₁₀ or N⁺R₁₀R₁₀,
where each R₁₀ is independently H, alkyl, substituted alkyl, alkenyl, substituted alkenyl, alkynyl, substituted alkynyl, aryl, 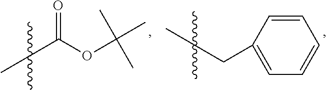
—CH₂CN, —CH₂CO₂R₁₂, or —CH₂COR₁₂,
where R₁₂ is H or alkyl,
or a salt, zwitterion or ester thereof.

47. The method of claim 46, wherein the compound has the structure 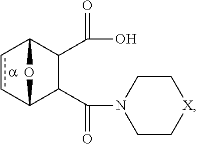
wherein:
bond α is present or absent;
X is O or NR₁₀,
where each R₁₀ is independently H, alkyl, substituted alkyl, alkenyl, substituted alkenyl, alkynyl, substituted alkynyl, aryl, 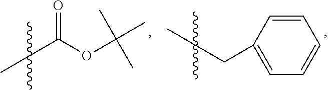
—CH₂CN, —CH₂CO₂R₁₂, or —CH₂COR₁₂,
where R₁₂ is H or alkyl,
or a salt, zwitterion or ester thereof.

48. The method of claim 46, where in the compound has the structure 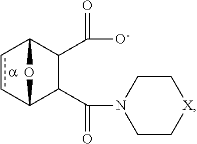
wherein:
bond α is present or absent;
X is O or NH⁺R₁₀,
where R₁₀ is H, alkyl, substituted alkyl, alkenyl, substituted alkenyl, alkynyl, substituted alkynyl, aryl, 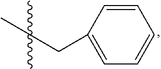 [SMILES: *Cc1ccccc1]
—CH₂CN, —CH₂CO₂R₁₂, or —CH₂COR₁₂,
where R₁₂ is H or alkyl,
or a salt, zwitterion or ester thereof.

49. The method of claim 40, wherein the compound has the structure 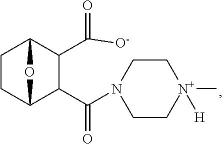 [SMILES: C[NH+]1CCN(C(=O)C2C(C(=O)[O-])[C@H]3CC[C@@H]2O3)CC1]
or a salt or ester thereof.
